(19) United States
(12) Patent Application Publication
(10) Pub. No.: US 2025/0262192 A1
(43) Pub. Date: Aug. 21, 2025

(54) PROGNOSTIC PATHWAYS FOR HIGH RISK SEPSIS PATIENTS

(71) Applicant: KONINKLIJKE PHILIPS N.V., EINDHOVEN (NL)

(72) Inventors: Wilbert Hendrik BOUWMAN, HEDEL (NL); Anja VAN DE STOLPE, VUGHT (NL)

(21) Appl. No.: 18/280,561

(22) PCT Filed: Mar. 9, 2022
(86) PCT No.: PCT/EP2022/056083

§ 371 Date: Sep. 6, 2023

(30) Foreign Application Priority Data
Mar. 11, 2021 (WO) PCT/EP2021/056133

(51) Int. Cl. A61K 31/437; A61K 31/167; A61K 31/4709; A61K 31/58; A61K 38/17; A61P 37/06

(52) U.S. Cl. CPC A61K 31/437; A61K 31/167; A61K 31/4709; A61K 31/58; A61K 38/1793; A61P 37/06; C07K 2319/30

(57) ABSTRACT

The present invention relates to sepsis, and to compounds useful in the prevention and treatment of subjects suffering from sepsis or at risk for developing sepsis. The invention particularly relates to compounds that inhibit the AR cellular signaling pathway activity, or compound that inhibit the TGFbeta cellular signaling pathway activity. The invention further relates to methods of measuring AR and/or TGFbeta pathway activity in a blood sample of a subject at risk of developing sepsis or suffering from sepsis, and administering an AR and/or a TGFbeta pathway inhibitor when the AR and/or the TGFbeta pathway activity exceeds a certain threshold.

### FIELD OF THE INVENTION

The present invention relates to sepsis, and to compounds useful in the prevention and treatment of subjects suffering from sepsis or at risk for developing sepsis, or the prevention of the immune suppressive state of the immune system. The invention particularly relates to compounds that inhibit the AR cellular signaling pathway activity, and/or compound that inhibit the TGFbeta cellular signaling pathway activity. The invention further relates to methods of measuring AR and/or TGFbeta pathway activity in a blood sample of a subject at risk of developing sepsis or suffering from sepsis, and administering an AR and/or a TGFbeta pathway inhibitor when the AR and/or the TGFbeta pathway activity exceeds a certain threshold.

### BACKGROUND OF THE INVENTION

Sepsis has recently been redefined as: infection with organ dysfunction (10). Sepsis is a dysregulated immune response to infection. Sepsis is generally a complication of severe bacterial infection in which bacteria amplify in the bloodstream. This condition is characterized by a systemic inflammatory response, which through a not fully understood mechanisms can lead to septic shock with hypotension that is refractory to fluid resuscitation and hyperlactatemia, progressing organ failure and consequently death. Mortality rates from sepsis range between 25% to 30% for severe sepsis and 40% to 70% for septic shock. The clinical presentation of sepsis is highly variable depending on the etiology, that is, the underlying disease or condition. The most common bacterial sources of infection are the respiratory, genitourinary, and gastrointestinal systems, as well as the skin and soft tissue. Though clinical presentation can be variable, fever with extreme shivering and tachycardia is often the first clinical manifestation of sepsis, with pneumonia and urogenital infection being the most common causes leading to sepsis. Specific treatment consists primarily of giving antibiotics to treat the infection, in addition to standard measures to maintain the blood circulation and prevent hypoxia. However the bacterial cause of the infection is often unknown, so the appropriate antibiotic treatment is an educated guess, until information on the causal bacteria and their sensitivity/resistance pattern to antibiotics becomes available from blood cultures. This information enables adjustment of therapy in case the bacterial species turned out to be resistant to the administered antibiotics. However, blood cultures take long (24 to 48 hours) and turn positive in around 30% of all patients with infections, while, for patients with bacterial sepsis, each hour of delay of administering effective antibiotics increases the relative risk of mortality.

For these reasons many patients progress to septic shock, resulting in a very high mortality for this disease. Besides antibiotics, treatment of septic shock subjects generally consists of intravenous fluid administration and vasopressor therapy. Many additional treatments have been tried to reverse the inflammatory septic shock state, e.g. corticosteroids, however none has proven to be of clinical benefit, although it cannot be excluded that they may benefit a subset of patients who so far cannot yet be identified (10).

One of the reasons is that it is not clear which cellular mechanism(s) is/are responsible for the transition to septic shock and the organ damage. Knowledge on the pathophysiology of sepsis is expected to provide novel drug targets to prevent and treat sepsis in a rational manner.

Aside from antibiotics and supportive measures to maintain blood circulation of internal organs, no treatments have proven to be effective, although it cannot be excluded that some treatments may benefit a subset of patients who so far cannot be identified [10]. One reason for failure to develop effective treatments is the heterogeneity among sepsis patients, that is, variation in underlying medical conditions and use of drugs, and genetic variations influencing the immune response in an individual patient.

One of the reasons for failure to develop an effective drug for all is probably the heterogeneity among patients, resulting from the large number of causes and conditions that can cause sepsis and the even larger number of known and unknown factors that influence its progression, while all together determine response or resistance to a drug (e.g. type of bacterial species, genetics, co-existing other diseases, etc.).

Detailed assessment of the functional immune response in a patient with sepsis may enable novel general and personalized treatment approaches and improve treatment efficacy. Diagnostic assessment of immune function is currently limited to routine blood measurements, such as numbers of immune cells and inflammation markers (e.g., C-reactive protein), but is not informative on the functional activity state of the various types of immune cells, responsible for the abnormal immune response in a sepsis patient.

The continuum of an inflammatory response to microorganisms is currently classified as infection, sepsis, and septic shock, replacing the older sequence: sepsis, severe sepsis, and septic shock. In addition systemic inflammatory response syndrome (SIRS) was a condition, defined by the presence of two out of four criteria: fever, tachycardia, tachypnea, and leukocytosis or leukopenia, in the absence of (proven) infection: more recently this diagnosis has been abandoned because of lack of sensitivity and specificity.

Clinical criteria used to identify sepsis patients in the hospital who are at high risk of sepsis-related death are (1) changed mental state: (2) systolic blood pressure <100 mmHg, and respiratory rate over 22 per minute. Using these criteria enables improved identification of high-risk patients, but cannot identify patients at low risk.

Rationally the best way to reduce death from sepsis is to as early as possible recognize patients at risk for developing sepsis. Such a high-risk patient can subsequently be stratified for more intense monitoring (for example at an ICU), identification and elimination of infectious foci (e.g. indwelling catheters), performing more frequent bacterial cultures, administration of preventive antibiotics, and elimination of immunosuppressive factors.

Urinary catheters, open wounds or wounds with drains, intravascular lines etc. are all nearly invariably associated with some extent of bacterial colonization, and therefore may present a risk at sepsis in a susceptible patient. These conditions are present in a major part of the hospitalized patients, and as such they represent a patient population in which clinical outcome may be improved upon early assessment of risk at sepsis and septic shock. Patient genetics and the functional state of the immune system are likely to be relevant factors determining risk.

Susceptibility of a patient with an infection to develop sepsis and progress to septic shock, is in part determined by the immune response that the patient mounts against the infectious pathogen.

The functionality of the immune response (immune active versus immune suppressed, inflammatory immune response versus adaptive T-cell mediated response) is determined by multiple endogenous factors, such as age, genetic variations, comorbidities (e.g. chronic diseases like diabetes), past treatments (like chemotherapy, bone marrow transplantation, corticosteroids), and exogenous factors such as current immunosuppressive treatment.

The consequence of all these factors on the functioning of the immune system in an individual patient cannot yet be assessed. This makes it currently very difficult to predict (1) which patient with an infection will be at risk of developing sepsis, (2) what is the size of that risk, (3) what is the risk at progression to septic shock and ultimately death. Realizing that the state of the immune response is an important determinant of the risk to develop sepsis, may also enable development of novel therapies for patient with an infection or sepsis, based on improving the failing immune function (i.e. immunotherapy).

However due to the large heterogeneity among patients, including gene variants that influence both the immune response and the susceptibility to develop sepsis, the molecular cause for the dysfunctional immune response in sepsis differs between individuals. This implicates that a personalized treatment approach will be necessary, based on characterization of the cellular mechanisms which determine the functional immune status in the individual patient, in order to choose the most effective immune targeting drug with minimal side effects (e.g. a cytokine storm). Personalized treatment based on the genetic profile of a patient has not proven to be effective.

Clearly the immune dysfunction phenotype that is involved in the susceptibility for sepsis is determined not only by the genetic profile, but also to an important extent by the many environmental cues of the patient (e.g. bacterial species, load, comorbidities, medication etc.). This implies that the individual functional immune status should be characterized on a phenotypic level, that is activity of cellular mechanisms which control immune functions.

Immune cell functions are orchestrated by highly controlled interactions between signaling pathways, like the TGFbeta, PI3K, MAPK, JAK-STAT, and AR pathways. Recently novel assays were developed to measure activity of signal transduction pathways in a cell or tissue sample, including a blood sample, based on target gene mRNA measurements, that are interpreted by a Bayesian computational models and translated into a pathway activity score (2), (3), (4). Measuring activity of these pathway allows characterization of the functional status of all types of immune cells (5).

The diagnosis sepsis is based on partially subjective clinical criteria, and therefore is not very sensitive, nor specific, mainly due to the large variety in patients (1). When sepsis is suspected based on clinical criteria, it remains important to rapidly confirm the diagnosis, in order to work out the patient care and treatment plan. Confirmation by for example a blood culture is a time-consuming process which can take several days, and cannot be waited for.

Faster confirming diagnostic tests are needed, as well as tests to predict which patient with an infection (mainly in the hospital) is at risk of developing sepsis, test to predict the risk to proceed to septic shock, and to predict risk at death. The clinical action that can be taken based on such a test can for example be to stratify subjects for admission to an intensive care unit (ICU) versus remaining at the general ward, to stratify for surgical search for and removal of a source of infection in a high risk patient, to stratify for specific treatment, e.g. targeted immunotherapy.

To summarize, there is a high need for additional tests which can be rapidly performed and can be used to (1) predict sepsis risk in an infected hospital (or at home) patient, to (2) early diagnose sepsis, and to (3) predict risk at progression to septic shock and death; and (4) to predict response to therapy, such as (targeted) therapy or immunotherapy on an individual patient basis (personalized treatment). Additionally there is an urgent need for (5) additional methods of treatment or prevention of sepsis.

### SUMMARY OF THE INVENTION

In accordance with a first aspect of the invention, the above problem is solved by an AR cellular signaling pathway inhibitor for use in the prevention or treatment of sepsis in a subject.

In accordance with a second aspect of the invention, the above problem is solved by a TGFbeta pathway inhibitor for use in the prevention or treatment of sepsis in a subject.

### DETAILED DESCRIPTION OF THE EMBODIMENTS

The functional state of immune cells is determined by a small number of so-called cellular signal transduction pathways (STPs) ([20], [21], [22], [23], [24]; each of which are hereby incorporated by reference in its entirety). Recently, novel assays have been developed to quantitatively measure activity of STPs in cell and tissue samples, including blood samples ([25], [26], [27], [28]; each of which are hereby incorporated by reference in its entirety). Measuring combined activity of these STPs in blood cells is expected to enable quantitative assessment of the innate and adaptive immune response in an individual.

STP analysis was performed on publicly available gene expression data from multiple clinical sepsis studies ([30], incorporated by reference in its entirety). Studies described herein show that activity of the AR and TGFbeta was increased in sepsis patients compared to healthy controls, indicating that these pathways represent novel drug targets to treat or prevent sepsis.

Treatment with a pathway inhibitor, particular an AR or an TGFbeta pathway inhibitor, or combinations thereof, may be beneficial for subjects having sepsis or in danger of developing sepsis. Treatment of sepsis with AR inhibitors has been tried before in a murine model but was unsuccessful. This may in part be due to gender based differences in androgen and/or testosterone levels, and consequent activity of the AR pathway. From this study it was concluded that treatment of sepsis with AR inhibitors was not viable. The present disclosure now demonstrates that first of all patients can be stratified by AR pathway activity, allowing treatment of only those patients with strongly increased AR pathway activity. More importantly, patients with an infection who are at risk of developing sepsis and have high AR pathway activity in relevant blood cells can benefit from preventive treatment with an AR inhibitor. Such preventative treatment with AR inhibitors has not been suggested as far as the inventors are aware. The rationale behind this is that an active AR pathway results in immunosuppression, see e.g. Gubbels Bupp and Jorgensen 2018 [5], incorporated by reference in its entirety.

It was found that AR proteins are expressed in a wide variety of innate and adaptive immune cells including neutrophils, macrophages, mast cells, monocytes, megakaryocytes, B cells, and T cells, suggesting that the AR pathway may indeed be ligand-inducible, which the inventors herein confirm for monocytic cells, the main cell type held responsible for the sepsis symptomatology. Interestingly, AR proteins are expressed also in hematopoietic stem cells and lymphoid and myeloid progenitor cells. Evidence derived from different studies points to an immunosuppressive role of androgens in different immune cell types mostly by changing expression of pro-inflammatory and anti-inflammatory mediators which are important for an appropriate immune response.

Concurrent with the pro-inflammatory response in sepsis, there is evidence of a number of immune suppressive events occurring in which immune suppressive cytokines such as IL-10, TGF-β, and IL6 play a role. According to a clinical observational cohort study, septic patients can develop chronic critical illness, with a 6-month survival of 63%, and continue to demonstrate cytokine profiles of chronic inflammation, as well as biomarker profiles characteristic for persistent immunosuppression [31]. In the study from Hiraki et al. [32], a murine abdominal sepsis model was utilized in which a TGF-β depleting antibody was administered leading to improved survival of the mice. This suggests a causal role for the TGFbeta pathway in sepsis. So far treatment or prevention with TGFbeta inhibitors has not been suggested.

The present invention is further based on the inventors' innovation that analysis of signal transduction pathway activities can be used to characterize a blood sample, for example a blood sample consisting of at least one immune cell type or a mixed collection of immune cell types, based on determining the activity of for example the AR signaling pathway, and optionally determining the activity or activities of additional or alternative pathway(s), such as the TGFbeta pathway, the MAPK-AP1 pathway and the JAK-STAT3 pathway. The inventors demonstrate that based on the AR pathway activity alone, when determined on a blood sample such as a whole blood sample, it can function as a test to establish or confirm the diagnosis of sepsis in a patient with clinical criteria that are suggestive of sepsis. When a low AR pathway activity is found, sepsis may still be present, but the patient is likely to be a survivor. Further, the level of AR pathway activity can be used to distinguish between low and high mortality risk, meaning based on blood samples obtained from subjects having sepsis a distinction can be made between samples obtained from a subject having high mortality risk and samples obtained from a subject having low mortality risk, based on the AR pathway activity. Lastly it was found that the correlation between pathway activity and sepsis vs control or low vs high mortality risk was specific for these four pathways, no other signaling pathways were found to correlate with presence or absence of sepsis or severity of sepsis (including mortality risk) in whole blood samples (data not shown).

Two of the key pathways that are elevated in sepsis, and highly indicative of poor prognosis, are the AR pathway and the TGFbeta pathway. These pathways also demonstrate increased activity levels in the pre-septic state. Therefore, the inventors next investigated whether these pathways are causative, and thus may be used to prevent or treat sepsis in patients, by interfering with the AR and/or the TGFbeta pathway activity.

In view of the clinical heterogeneity between sepsis patients, as already shown before, personalized treatment may offer an additional advantage. Until now it was not possible to stratify patients that would benefit from AR or TGFbeta pathway inhibitors for prevention or treatment of sepsis. The methods described herein now allow for an accurate assessment of the AR or TGFbeta pathway activity in a blood sample. This allows discrimination between patients with low and high AR and/or TGFbeta pathway activity and administering AR and/or TGFbeta inhibitors to those with high AR and/or TGFbeta pathway activity in order to normalize pathway activity.

The inventors therefore theorized that based on the below described findings, treatment with a pathway inhibitor, particular an AR and/or TGFbeta pathway inhibitor, may be beneficial for subjects having sepsis or in danger of developing sepsis. Treatment of mice with sepsis with AR inhibitors has been described before with mixed success [16]. It was found that male mice with sepsis benefit from treatment with AR inhibitors but not female mice with sepsis. It should be noted however that there is strong gender-based difference in response to sepsis (with a much higher mortality rate in male mice), therefore it is not very clear to what extent these results can be extrapolated to humans. Based on our results any difference in response to treatment with an AR and/or TGFbeta inhibitor can be explained by the fact that not all sepsis patients exhibit high AR and/or TGFbeta pathway activity, and therefore only those with strongly increased AR and/or TGFbeta pathway activity would benefit from treatment with an AR and/or TGFbeta pathway inhibitor. More importantly, patients with an infection who are at risk of developing an infection and exhibit a high AR and/or TGFbeta pathway activity may benefit the most from preventative treatment with an AR and/or TGFbeta inhibitor. The rationale behind this assumption is that an active AR and/or TGFbeta pathway results in immunosuppression, see e.g. Gubbels Bupp and Jorgensen, Androgen-Induced Immunosuppression, Front Immunol. 2018; 9: 794 [11], incorporated by reference in its entirety.

It was found that AR are expressed in a wide variety of innate and adaptive immune cells including neutrophils, macrophages, mast cells, monocytes, megakaryocytes, B cells, and T cells. Interestingly, AR are expressed also in hematopoietic stem cells and lymphoid and myeloid progenitor cells [13]. Evidence derived from different studies points to a rather immunosuppressive role of androgens and TGFbeta in different immune cell types mostly by reducing and/or promoting expression of pro-inflammatory and anti-inflammatory mediators.

Until now it was not possible to stratify patients that would benefit from AR and/or TGFbeta pathway inhibitors for prevention or treatment of sepsis, however the methods described herein allow for an accurate assessment of the AR and TGFbeta pathway activity in a blood sample. This allows to simply discriminate between patients with low and high AR and/or TGFbeta pathway activity and administering AR and/or TGFbeta inhibitors to those with high AR and/or TGFbeta pathway activity.

In order to verify this hypothesis an in vitro experiment was designed where monocytes were stimulated with LPS and subsequently treated with a variety of AR and/or TGFbeta pathway inhibitors. Monocytes were chosen as they have been described to play a major role in sepsis, see for example Sukhacheva, The role of monocytes in the progression of sepsis, Clinical Laboratory Int. 26 Aug. 2020 [14] or Haverman et al., The central role of monocytes in the pathogenesis of sepsis: consequences for immunomonitoring and treatment, The Netherlands Journal of Medicine, Volume 55, Issue 3, September 1999, Pages 132-141 [15], both incorporated by reference in its entirety. The inventors were able to demonstrate that (bacterial) LPS was able to stimulate AR and TGFbeta signaling pathway in monocytes, and that this effect could be largely counteracted by incubating the monocytes with an AR and/or TGFbeta pathway inhibitor after stimulation.

Since it is known that monocytes are one of the key cell types to play a role in sepsis, that AR signaling activity is increased and this activity can measured in monocytes, and AR and TGFbeta signaling are known to have an immunosuppressing role as well as an inflammatory role, it is entirely plausible that treatment of patients with high AR and/or TGFbeta pathway activity leads to prevention or successful treatment of sepsis, or at least mitigates the symptoms. Example 10 provides a first proof of concept that AR pathway inhibitors can be used to treat or prevent sepsis by using a THP-1 cell model. The data demonstrates that LPS induces both AR and TGFbeta pathway activity in THP-1 monocyte cells, and that AR pathway activity levels can be reverted almost to baseline levels by addition of an AR pathway inhibitor.

Example 11 provides data to further support that AR inhibitors and TGFbeta inhibitors can be used for the prevention and treatment of sepsis. In order to support this, a prevention and a treatment model were designed to test AR and TGFbeta inhibitors. In these models a monocytic cell line (THP-1) is used as monocytes are deemed one of the relevant cells in the septic response and have been demonstrated to provide an upregulation of both AR and TGFbeta pathway activity in septic patients (see previous Examples).

In the prevention model, cells were pre-incubated with pathway inhibitors or a negative control (DMSO) for 24 hours and then incubated for 24 hours with LPS (or dihydrotestosterone (DHT) as a positive control). Then RNA was extracted from the cells and pathway activity was determined by the methods described herein. These data are described in Tables 7, 8 and 10.

In the treatment model the pathway inhibitor and LPS were added at the same time and the THP-1 cells were incubated for 24 hours prior to isolating RNA and determining pathway activities. These data are described in Tables 9A and 11.

To demonstrate that the effect of increased AR and TGFbeta pathway activity induced by LPS is cell type specific, a comparison was made between monocytic cells (THP-1) and a lymphoid cell line (MOLT-4). These data can be seen in Tables 9A and 9B, and demonstrate that LPS causes activation of both AR and TGFbeta pathway activity in THP-1 cells, but that MOLT-4 do not display any changes in pathway activation. In THP-1 cells the activity could be slightly reduced by adding either bicalutamide or enzalutamide, however it is to be noted that the used concentration of LPS is extremely high, explaining the only minor effect of these pathway inhibitors in this particular experiment.

Next it was reviewed whether an effect can be observed on PBMCs from healthy subjects as well. As PBMCs only contain 5-10% monocytes, it was expected that effects are only minor. Indeed the data in Tables 12A and 12B demonstrate that LPS did not significantly upregulate AR or TGFbeta pathway activity in PBMCs obtained from healthy volunteers, likely due to the low percentage of monocytes where an effect is expected. Nonetheless a minor reduction in both AR and TGFbeta pathway activities was observed when LPS was combined with (R)-bicalutamide, and this effect could be increased dramatically by also including Vactosertib.

Despite the above, PBMCs from patients with sepsis were obtained and analyzed, untreated and treated with different pathway inhibitors. AR and TGFbeta pathway activities were generally found to be elevated in sepsis patients, and could surprisingly be reduced by incubating the PBMCs with AR and/or TGFbeta pathway inhibitors for 24 hours, providing further evidence that AR and TGFbeta cellular signaling pathway inhibitors may be used in the treatment of sepsis.

To summarize, examples 1-9 provide evidence that several pathways are upregulated in patients with sepsis or patients at risk of developing sepsis, and moreover that pathway analysis allows to stratify patients, for example for selecting those patients at highest risk of a fatal outcome.

Therefore, in a first aspect, the invention relates to an AR cellular signaling pathway inhibitor for use in the prevention or treatment of sepsis in a subject. Alternatively the invention relates the a method of preventing or treating sepsis in a subject in need thereof, comprising administering an AR cellular signaling pathway inhibitor to the subject.

When used herein prevention or treatment of sepsis by a pathway inhibitor refers to administering a pathway inhibitor to a subject in need thereof, meaning a subject having sepsis or at risk of developing sepsis. Routes of administration are generally known to the skilled person and dependent on the compound. Therefore the methods and uses described herein are not limited to a specific route of administration and may for example refer to enteral, parenteral or topical administration. Examples of enteral administration routes that may be used include oral, rectal sublingual, sublabial or buccal administration, wherein oral administration is preferred. Parenteral administration may include Epidural,
Intracerebroventricular, Epicutaneous, Sublingual or buccal, Sublingual, Nasal, Intra-arterial, Intra-articular, Intracardiac, Intracavernous, Intralesional, Intramuscular, Intraocular, Intraosseous, Intraperitoneal, Intrathecal, Intravaginal, Intravenous, Intravesical, Intravitreal,
Subcutaneous, Transdermal, Perivascular or Transmucosal administration, for example by injection needle. Preferred parenteral routes are subcutaneous or intramuscular administration.

When used herein, prevention refers to a situation where a subject is deemed to not have sepsis but is at high risk of developing sepsis, wherein prevention is assumed to mean reducing the chance of said subject to develop sepsis. When used herein treatment refers to a situation wherein a subject has sepsis and wherein the treatment may refer to reducing symptoms, shortening the time the subject is septic reducing the change of mortality or curing the subject, compared to a situation where the subject has not received the respective treatment. Treatment may also refer to reducing the immunosuppressed state of a subject.

The subject may be human or a non-human mammal. Preferably the subject is human. In an embodiment the subject has an elevated AR cellular signaling pathway activity or wherein the AR cellular signaling pathway activity exceeds a threshold. Alternatively the invention relates the a method of preventing or treating sepsis, comprising administering an AR cellular signaling pathway inhibitor to a subject having an elevated AR cellular signaling pathway activity or a subject having the AR cellular signaling pathway activity exceed a threshold.

When used herein, elevated, when referring to a pathway activity, should be interpreted as meaning higher or increased when compared to a baseline or control level. For example, pathway activities such as the AR or the TGFbeta pathway activities, can be determined on blood samples of a cohort of healthy individuals to establish a mean pathway activity value as the baseline or control level. The pathway activity measured in the blood sample of the subject may then simple be compared to this mean value. Because there will be natural variation in the observed pathway activities in the healthy cohort, a threshold can be set above which the pathway activity is said to be increased or elevated. Similarly a threshold can be set below which the pathway activity is said to be decreased or lower. For example when using pathway activities obtained from a cohort of healthy subjects, the thresholds can be set using the mean pathway activity plus or minus one times the standard deviation. A more strict threshold can be set by making the threshold the mean plus or minus two times, three times or even four times the standard deviation of the pathway activities obtain from the blood samples of the healthy cohort.

In an embodiment the AR cellular signaling pathway is determined in a blood sample obtained from the subject. The pathway activity may be determined on whole blood or isolated blood cells such as but not limited to PBMCs, monocytes or neutrophils. Alternatively the invention relates the a method of preventing or treating sepsis, comprising administering an AR cellular signaling pathway inhibitor to a subject having an elevated AR cellular signaling pathway activity or a subject having the AR cellular signaling pathway activity exceed a threshold, wherein the AR cellular signaling pathway is determined in a blood sample obtained from the subject.

In an embodiment the AR cellular signaling pathway inhibitor is administered if the AR cellular signaling pathway activity determined in the blood sample of the subject is found to be elevated or to exceed a certain threshold. The threshold may be predetermined. The skilled person will be aware that the numerical value obtained and representing the pathway activity depends on the method used. Therefore the same method is ideally used to determine the pathway activity, such as the AR pathway activity, in the blood sample of the subject and in the blood samples of the references, such as for example healthy subjects.

Therefore, in an embodiment the invention relates to an AR cellular signaling pathway inhibitor for use in preventing or treating sepsis, the use comprising:
determining the AR cellular signaling pathway activity in a blood sample of the subject; and
administering an AR cellular signaling pathway inhibitor to the patient if the AR cellular signaling pathway activity in the blood sample of the subject is found to be elevated or to exceed a certain threshold.

The skilled person is aware that there are a multiple methods available to determine AR cellular signaling pathway activity. For example in the uses and methods described herein, AR pathway activity may be determined by a reporter assay, nuclear staining of the receptor transcription complex, phosphorylation based assays, ELISA, or based on markers or target genes as described herein. The skilled person is however aware that other methods may be used, therefore this list should not be interpreted as limitative. Thus the methods and uses described herein are not limited to particular methods of determining pathway activity. For example the methods described herein below may be used. Therefore, in an embodiment, the determining the AR cellular signaling pathway comprises: determining or receiving the expression levels of three or more target genes of the AR signaling pathway in the blood sample of the subject; determining an activity level of the AR cellular signaling pathway associated transcription factor (TF) element in the sample, the TF element controlling transcription of the three or more target genes, the determining being based on evaluating a calibrated mathematical pathway model relating expression levels of the three or more target genes to the activity level of the AR cellular signaling pathway, and inferring the activity of the AR cellular signaling pathway in the blood sample from the subject based on the determined activity level of the AR cellular signaling pathway associated TF element. Details on the method of inferring a pathway activity based on determining an activity level of a TF element are provided below.

When used herein, the term AR cellular signaling pathway inhibitor is used interchangeably with the term AR pathway inhibitor, and used to describe a compound or biological that inhibits, reduces or decreases the transcription of target genes by the activated Androgen Receptor complex. The AR pathway inhibitor may be a direct inhibitor or an indirect inhibitor, meaning it may directly interfere in the signaling cascade of AR cellular signaling or indirectly by interfering in a pathway that regulates the AR cellular signaling pathway more downstream. For example, the inhibitor may function by making a ligand for the AR pathway such as testosterone unavailable, interfering with reduction of the metabolic step of testosterone to DHT, interfering with translocation of activated AR to the nucleus or by interfering with initiation of transcription by the AR transcription complex, or by reducing AR expression, for example by AR degradation. For example, etanercept (also known as Enbrel) is mainly known as a compound for interfering with tumor necrosis factor (TNF), however in our data we observe significant reduction of both AR and TGFbeta pathway signaling when this compound is used in a relevant model system, therefore for the purpose of the present invention etanercept is considered an AR pathway inhibitor as well as a TGFbeta pathway inhibitor. For example, resatorvid (also known as TAK-242) is mainly known as an antagonist of the Toll-like receptor 4 (TLR4), however in our data we observe significant reduction of both AR and TGFbeta pathway signaling when this compound is used in a relevant model system, therefore for the purpose of the present invention resatorvid is considered an AR pathway inhibitor as well as a TGFbeta pathway inhibitor. For example, filgotinib (also known as Jyseleca) is mainly known as a JAK1 inhibitor, however in our data we observe significant reduction of both AR and TGFbeta pathway signaling when this compound is used in a relevant model system, therefore for the purpose of the present invention filgotinib is considered an AR pathway inhibitor as well as a TGFbeta pathway inhibitor. Therefore when used herein, an AR inhibitors is any compound or biological that is capable of inhibiting AR pathway activity in a relevant model system such as but not limited to THP-1 cells, where the AR pathway activity is inferred by the model as described herein. Secondary effects of AR pathway inhibitors can be changes in activity of other signaling pathways, for example by the production of intermediate proteins, or interactions between transcription factors of the various signaling pathways.

Therefore, in an embodiment, the AR cellular signaling pathway inhibitor is selected from the group consisting of Steroidal antiandrogens, Nonsteroidal antiandrogens, Androgen synthesis inhibitors, CYP17A1 inhibitors, CYP11 A1 (P450scc) inhibitors, 5α-Reductase inhibitors and Antigonadotropins or combinations thereof. Alternatively the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), and Progestogens (e.g., chlormadinone acetate, cyproterone acetate, gestonorone caproate, medroxyprogesterone acetate, megestrol acetate), etanercept, resatorvid, filgotinib or combinations thereof. In a more preferred embodiment the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), etanercept, resatorvid, filgotinib or combinations thereof.

In an embodiment, the invention relates to an AR cellular signaling pathway inhibitor for use in the prevention or treatment of sepsis in a subject, as described herein wherein the use is for the prevention of sepsis in a subject at risk of developing sepsis.

In an embodiment, the invention relates to an AR cellular signaling pathway inhibitor for use in the prevention or treatment of sepsis in a subject as described herein, wherein the use is for the treatment of sepsis in a subject suffering from sepsis.

It may be particularly advantageous to combine an AR inhibitor with an TGFbeta inhibitor to prevent or treat sepsis in a subject. Therefore, in an embodiment, the AR cellular signaling pathway inhibitor is administered together with a TGFbeta cellular signaling pathway inhibitor, wherein the AR cellular signaling pathway inhibitor and the TGFbeta cellular signaling pathway inhibitor are the same compound or a different compound.

The AR and TGFbeta inhibitors may be combined in a single therapeutic or may be administered separately. Therefore, in an embodiment the AR cellular signaling pathway inhibitor is administered prior to the TGFbeta cellular signaling pathway inhibitor, or wherein the AR cellular signaling pathway inhibitor is administered simultaneously with the TGFbeta cellular signaling pathway inhibitor, or wherein the AR cellular signaling pathway inhibitor is administered after the TGFbeta cellular signaling pathway inhibitor.

Activation of the AR and TGFbeta cellular signaling pathway in sepsis and subjects at risk of developing sepsis varies. Therefore, some subjects may benefit more from an AR and/or a TGFbeta cellular signaling pathway inhibitor. It may therefore be beneficial to determine the AR and/or the TGFbeta pathway activities in a blood sample from the subject prior to treatment. Therefore in an embodiment, the invention relates to an AR cellular signaling pathway inhibitor for use in the prevention or treatment of sepsis in a subject, wherein the use comprises:
determining the AR cellular signaling pathway activity and the TGFbeta cellular signaling pathway activity in a blood sample of the subject; and
administering an AR pathway inhibitor to the patient when the AR cellular signaling pathway activity is found to be elevated or to exceed a certain threshold; and
administering an TGFbeta pathway inhibitor to the patient when the TGFbeta cellular signaling pathway activity is found to be elevated or to exceed a certain threshold.

For example when a high AR pathway and a low TGFbeta pathway activity are measured in the blood sample of the subject, an AR pathway inhibitor may be administered, or when a high AR pathway and a high TGFbeta pathway activity are measured in the blood sample of the subject, both an AR and a TGFbeta pathway inhibitor may be administered. Alternatively, when a low AR pathway and a high TGFbeta pathway activity are measured in the blood sample of the subject, a TGFbeta pathway inhibitor may be administered.

Therefore, in an embodiment, the TGFbeta cellular signaling pathway inhibitor is selected from the group consisting of Small molecule kinase inhibitors, Anti-TGF-β ligand antibodies, Anti-TpR receptor antibodies or Antisense oligonucleotides or combinations thereof, or
wherein the TGFbeta cellular signaling pathway inhibitor is selected from the group consisting of Vactosertib (EW-7197), Galunisertib (LY2157299), LY3200882, (E)-SIS3, Fresolimumab, XPA681, XPA089, LY2382770, LY3022859, ISTH0036, ISTH0047, Pyrrole-imidazole polyamides, etanercept, resatorvid, filgotinib or combinations thereof,
preferably selected from the group consisting of Vactosertib (EW-7197), Galunisertib (LY2157299), LY3200882, (E)-SIS3, etanercept, resatorvid, filgotinib or combinations thereof.

In an embodiment the use is for prevention of sepsis and the AR inhibitor is bicalutamide and/or the TGFbeta inhibitor is vactosertib.

In an embodiment the use is for treatment of sepsis and the AR inhibitor is bicalutamide, (R)-bicalutamide, Ketodarolutamide (ORM-15341), D4-abiraterone, Etanercept, Resatorvid, N-desmethyl Enzalutamide, N-Desmethyl-Apalutamide, or filgotinib or combinations thereof and/or the TGFbeta inhibitor is vactosertib, Etanercept, Resatorvid or filgotinib or combinations thereof.

In an embodiment the AR inhibitor is (R)-bicalutamide and the TGFbeta inhibitor is etanercept, or the AR inhibitor is (R)-bicalutamide and the TGFbeta inhibitor is filgotinib. Preferably the use is for treatment of sepsis and the AR inhibitor is (R)-bicalutamide and the TGFbeta inhibitor is etanercept, or the AR inhibitor is (R)-bicalutamide and the TGFbeta inhibitor is filgotinib. Alternatively, the use is for treatment of sepsis and the AR inhibitor is combined with etanercept or filgotinib, preferably wherein the AR inhibitor is (R)-bicalutamide.

In an embodiment, the invention relates to a compound for use in the prevention or treatment of sepsis. Alternatively the invention relates to a method of preventing sepsis in a subject or treating a subject with sepsis comprising administering a compound to the subject. The compound is preferably selected from Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), Progestogens (e.g., chlormadinone acetate, cyproterone acetate, gestonorone caproate, medroxyprogesterone acetate, megestrol acetate), etanercept, resatorvid, filgotinib, Vactosertib (EW-7197), Galunisertib (LY2157299), LY3200882, (E)-SIS3, Fresolimumab, XPA681, XPA089, LY2382770, LY3022859, ISTH0036, ISTH0047, Pyrrole-imidazole polyamides or combinations thereof.

In an embodiment, the invention relates to an AR cellular signaling pathway inhibitor for use in the prevention or treatment of sepsis in a subject, as described herein wherein the use is for the prevention of sepsis in a subject at risk of developing sepsis.

In an embodiment, the invention relates to an AR cellular signaling pathway inhibitor for use in the prevention or treatment of sepsis in a subject as described herein, wherein the use is for the treatment of sepsis in a subject suffering from sepsis.

In a second aspect, the invention relates to a TGFbeta pathway inhibitor for use in the prevention or treatment of sepsis in a subject. Alternatively the invention relates the a method of preventing or treating sepsis in a subject in need thereof, comprising administering an TGFbeta cellular signaling pathway inhibitor to the subject. In an embodiment the subject has an elevated TGFbeta cellular signaling pathway activity or wherein the TGFbeta cellular signaling pathway activity exceeds a threshold. Alternatively the invention relates the a method of preventing or treating sepsis, comprising administering an TGFbeta cellular signaling pathway inhibitor to a subject having an elevated TGFbeta cellular signaling pathway activity or a subject having the TGFbeta cellular signaling pathway activity exceed a threshold.

In an embodiment the TGFbeta cellular signaling pathway is determined in a blood sample obtained from the subject. The pathway activity may be determined on whole blood or isolated blood cells such as but not limited to PBMCs, monocytes or neutrophils. Alternatively the invention relates the a method of preventing or treating sepsis, comprising administering an TGFbeta cellular signaling pathway inhibitor to a subject having an elevated TGFbeta cellular signaling pathway activity or a subject having the TGFbeta cellular signaling pathway activity exceed a threshold, wherein the TGFbeta cellular signaling pathway is determined in a blood sample obtained from the subject.

In an embodiment the TGFbeta cellular signaling pathway inhibitor is administered if the TGFbeta cellular signaling pathway activity determined in the blood sample of the subject is found to be elevated or to exceed a certain threshold. The threshold may be predetermined. The skilled person will be aware that the numerical value obtained and representing the pathway activity depends on the method used. Therefore the same method is ideally used to determine the pathway activity, such as the TGFbeta pathway activity, in the blood sample of the subject and in the blood samples of the references, such as for example healthy subjects.

Therefore, in an embodiment the invention relates to an TGFbeta cellular signaling pathway inhibitor for use in preventing or treating sepsis, the use comprising:
determining the TGFbeta cellular signaling pathway activity in a blood sample of the subject; and
administering an TGFbeta cellular signaling pathway inhibitor to the patient if the TGFbeta cellular signaling pathway activity in the blood sample of the subject is found to be elevated or to exceed a certain threshold.

The skilled person is aware that there are multiple methods available to determine TGFbeta cellular signaling pathway activity. For example in the uses and methods described herein TGFbeta pathway activity may be determined by a reporter assay, nuclear staining of the receptor transcription complex, phosphorylation based assays, ELISA. The skilled person is however aware that other methods may be used, therefore this list should not be interpreted as limitative. Thus the methods and uses described herein are not limited to particular methods of determining pathway activity. For example the methods described herein below may be used. Therefore, in an embodiment, the determining the TGFbeta cellular signaling pathway comprises: determining or receiving the expression levels of three or more target genes of the TGFbeta signaling pathway in the blood sample of the subject; determining an activity level of the TGFbeta cellular signaling pathway associated transcription factor (TF) element in the sample, the TF element controlling transcription of the three or more target genes, the determining being based on evaluating a calibrated mathematical pathway model relating expression levels of the three or more target genes to the activity level of the TGFbeta cellular signaling pathway, and inferring the activity of the TGFbeta cellular signaling pathway in the blood sample from the subject based on the determined activity level of the TGFbeta cellular signaling pathway associated TF element. Details on the method of inferring a pathway activity based on determining an activity level of a TF element are provided below.

When used herein, the term TGFbeta cellular signaling pathway inhibitor is used interchangeably with the term TGFbeta pathway inhibitor, and used to describe a compound or biological that inhibits, reduces or decreases the transcription of target genes by an activated complex of SMAD proteins. The TGFbeta pathway inhibitor may be a direct inhibitor or an indirect inhibitor, meaning it may directly in the signaling cascade of TGFbeta cellular signaling or indirectly by interfering in a pathway that regulates the TGFbeta cellular signaling pathway more downstream. For example, the inhibitor may function by making a ligand for the TGFbeta pathway such as TGFbeta unavailable or preventing it from binding to the TGFbeta receptor, interfering with the downstream signaling cascade leading to phosphorylation of SMAD proteins, interfering with translocation of activated SMAD protein complex to the nucleus or by interfering with initiation of transcription by the SMAD transcription complex. For example, etanercept (also known as Enbrel) is mainly known as a compound for interfering with tumor necrosis factor (TNF), however in our data we observe significant reduction of both AR and TGFbeta pathway signaling when this compound is used in a relevant model system, therefore for the purpose of the present invention etanercept is considered an AR pathway inhibitor as well as a TGFbeta pathway inhibitor. For example, resatorvid (also known as TAK-242) is mainly known as an antagonist of the Toll-like receptor 4 (TLR4), however in our data we observe significant reduction of both AR and TGFbeta pathway signaling when this compound is used in a relevant model system, therefore for the purpose of the present invention restorvid is considered an AR pathway inhibitor as well as a TGFbeta pathway inhibitor. For example, filgotinib (also known as Jyseleca) is mainly known as a JAK1 inhibitor, however in our data we observe significant reduction of both AR and TGFbeta pathway signaling when this compound is combined with (R)-bicalutamide in a relevant model system, therefore for the purpose of the present invention filgotinib is considered an AR pathway inhibitor as well as a TGFbeta pathway inhibitor. Therefore when used herein, a TGFbeta inhibitors is any compound or biological that is capable of inhibiting the TGFbeta pathway activity in a relevant model system such as but not limited to THP-1 cells, where the TGFbeta pathway activity is inferred by the model as described herein.

Therefore, in an embodiment, the TGFbeta cellular signaling pathway inhibitor is selected from the group consisting of Small molecule kinase inhibitors, Anti-TGF-β ligand antibodies, Anti-TOR receptor antibodies or Antisense oligonucleotides or combinations thereof alternatively the TGFbeta cellular signaling pathway inhibitor is selected from the group consisting of Vactosertib (EW-7197), Galunisertib (LY2157299), LY3200882, (E)-SIS3, Fresolimumab, XPA681, XPA089, LY2382770, LY3022859, ISTH0036, ISTH0047 Pyrrole-imidazole polyamides, etanercept, resatorvid, filgotinib or combinations thereof. In a preferred embodiment the TGFbeta cellular signaling pathway inhibitor is selected from the group consisting of Vactosertib (EW-7197), Galunisertib (LY2157299), LY3200882, (E)-SIS3, etanercept, resatorvid, filgotinib or combinations thereof.

In an embodiment, the invention relates to an TGFbeta cellular signaling pathway inhibitor for use in the prevention or treatment of sepsis in a subject, as described herein wherein the use is for the prevention of sepsis in a subject at risk of developing sepsis.

In an embodiment, the invention relates to an TGFbeta cellular signaling pathway inhibitor for use in the prevention or treatment of sepsis in a subject as described herein, wherein the use is for the treatment of sepsis in a subject suffering from sepsis.

It may be particularly advantageous to combine an AR inhibitor with an TGFbeta inhibitor to prevent or treat sepsis in a subject. Therefore in an embodiment, the TGFbeta pathway inhibitor is administered together with an AR pathway inhibitor, wherein the TGFbeta pathway inhibitor and the AR pathway inhibitor are the same compound or a different compound. The AR and TGFbeta inhibitors may be combined in a single therapeutic or may be administered separately. Therefore, in an embodiment the AR cellular signaling pathway inhibitor is administered prior to the TGFbeta cellular signaling pathway inhibitor, or wherein the AR cellular signaling pathway inhibitor is administered simultaneously with the TGFbeta cellular signaling pathway inhibitor, or wherein the AR cellular signaling pathway inhibitor is administered after the TGFbeta cellular signaling pathway inhibitor. In an embodiment, the invention relates to an AR cellular signaling pathway inhibitor for use in the prevention or treatment of sepsis in a subject, wherein the use comprises:
determining the AR cellular signaling pathway activity and the TGFbeta cellular signaling pathway activity in a blood sample of the subject; and
administering an AR pathway inhibitor to the patient when the AR cellular signaling pathway activity is found to be elevated or to exceed a certain threshold; and
administering an TGFbeta pathway inhibitor to the patient when the TGFbeta cellular signaling pathway activity is found to be elevated or to exceed a certain threshold.

In an embodiment the AR cellular signaling pathway inhibitor is selected from the group consisting of Steroidal antiandrogens, Nonsteroidal antiandrogens, Androgen synthesis inhibitors, CYP17A1 inhibitors, CYP11 A1 (P450scc) inhibitors, 5α-Reductase inhibitors and Antigonadotropins or combinations thereof. Alternatively the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), Progestogens (e.g., chlormadinone acetate, cyproterone acetate, gestonorone caproate, medroxyprogesterone acetate, megestrol acetate), etanercept, resatorvid, filgotinib or combinations thereof. In a more preferred embodiment the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), etanercept, resatorvid, filgotinib or combinations thereof.

In an embodiment, the invention thus relates to an AR and/or a TGFbeta pathway inhibitor for use in the prevention of sepsis in a subject suffering from an infection, preferably wherein the subject has an elevated AR and/or a TGFbeta cellular signaling pathway activity as determined in a blood sample obtained from the subject.

Optionally the AR and/or a TGFbeta cellular signaling pathway activity is determined on a blood sample obtained from the subject and the AR and/or a TGFbeta pathway inhibitor is administered if the AR and/or a TGFbeta cellular signaling pathway activity is found to be elevated or to exceed a certain threshold. The AR and/or a TGFbeta cellular signaling pathway may be determined using methods described herein, particularly the methods described in the first aspect of the invention.

Currently methods to prevent sepsis are aimed at reducing the underlying infection, such as administration of antibiotics. Using the methods described here it now is for the first time possible to identify and treat patients with an infection who are at risk of developing sepsis. By determining the AR and/or a TGFbeta activity of the subject in a blood sample, candidates for AR and/or a TGFbeta pathway inhibitor treatment can now easily be identified. A strong correlation was found with increased AR and/or a TGFbeta pathway activity and development of sepsis due to an infection. This is likely caused by the immunosuppressive action of AR and/or a TGFbeta pathway activity for example in monocytes. Monocytes have a strong inflammatory role in the innate immune response, and systemic inflammation is a hallmark of sepsis. Therefore, it is plausible that AR and/or a TGFbeta pathway activity is causative and thus inhibiting AR and/or a TGFbeta activity would increase the chance that sepsis is prevented or mitigated. Instead of determining the AR and/or a TGFbeta pathway activity the decision to administer an AR and/or a TGFbeta pathway inhibitor may also be based on determining the expression levels of three or more, for example three, four, five, six, seven, eight, nine, ten, eleven, twelve or more, AR target genes selected from group 1, wherein group 1 consists of the genes ABCC4, APP, AR, CDKN1A, CREB3L4, DHCR24, EAF2, ELL2, FGF8, FKBP5, GUCY1A3, IGF1, KLK2, KLK3, LCP1, LRIG1, NDRG1, NKX3_1, NTS, PLAU, PMEPA1, PPAP2A, PRKACB, PTPN1, SGK1, TACC2, TMPRSS2, and UGT2B15, preferably AR, CREB3L4, DHCR24, EAF2, ELL2, FKBP5, GUCY1A3, IGF1, KLK3, LCP1, LRIG1, NDRG1, NKX3_1, PMEPA1, PRKACB, TMPRSS2, more preferably AR, CREB3L4, DHCR24, EAF2, ELL2, FKBP5, LCP1, LRIG1, NDRG1, PMEPA1, PRKACB, TMPRSS2 even more preferably DHCR24, EAF2, ELL2, FKBP5, LCP1, LRIG1, PMEPA1, PRKACB and/or based on determining the expression levels of three or more, for example three, four, five, six, seven, eight, nine, ten, eleven, twelve or more TGFbeta target genes selected group 2, wherein group 2 consists of the genes ANGPTL4, CDC42EP3, CDKN1A, CDKN2B, CTGF, GADD45A, GADD45B, HMGA2, ID1, IL11, INPP5D, JUNB, MMP2, MMP9, NKX2_5, OVOL1, PDGFB, PTHLH, SERPINE1, SGK1, SKIL, SMAD4, SMAD5, SMAD6, SMAD7, SNAI1, SNAI2, TIMP1 and VEGFA, preferably CDC42EP3, GADD45A, GADD45B, HMGA2, ID1, IL11, INPP5D, JUNB, MMP2, MMP9, NKX2_5, OVOL1, PDGFB, PTHLH, SGK1, SKIL, SMAD4, SMAD5, SMAD6, TIMP1, VEGFA, more preferably CDC42EP3, GADD45A, GADD45B, ID1, JUNB, MMP9, PDGFB, SGK1, SKIL, SMAD5, SMAD6, TIMP1, VEGFA, even more preferably CDC42EP3, GADD45A, GADD45B, ID1, JUNB, MMP9, PDGFB, SGK1, SMAD5, TIMP1, VEGFA.

The invention further relates to an AR and/or a TGFbeta pathway inhibitor for use in the treatment or alleviation of a subject suffering from sepsis wherein the subject has an elevated AR and/or a TGFbeta cellular signaling pathway activity or an AR and/or a TGFbeta cellular signaling pathway activity exceeding a certain threshold as determined in a blood sample obtained from the subject.

Optionally the AR and/or a TGFbeta cellular signaling pathway activity is determined on a blood sample obtained from the subject and the AR and/or a TGFbeta pathway inhibitor is administered if the AR and/or a TGFbeta cellular signaling pathway activity is found to be elevated or to exceed a certain threshold. In an embodiment the AR and/or a TGFbeta pathway activity is determined by the methods described herein. Because TGFbeta pathway activity is also found to be elevated in patients with sepsis, it is speculated to inhibiting TGFbeta pathway activity together with AR pathway activity may beneficial. This may be achieved by administering an AR inhibitor and an TGFbeta inhibitor as two distinct compounds, or a single compound inhibiting both AR and TGFbeta can be used. For example, the compound A-458 was found to specifically inhibit both the AR and TGFbeta pathways and could be beneficially used for this purpose. Therefore, in an embodiment of the uses according to the invention, further the TGFbeta pathway activity is determined. In an embodiment the TGFbeta pathway activity is determined by the methods described herein.

Instead of determining the TGFbeta pathway activity the decision to administer an TGFbeta pathway inhibitor may also be based on determining the expression levels of three or more, for example three, four, five, six, seven, eight, nine, ten, eleven, twelve or more, genes selected from group 2, wherein group 2 consists of the genes ANGPTL4, CDC42EP3, CDKN1A, CDKN2B, CTGF, GADD45A, GADD45B, HMGA2, ID1, IL11, INPP5D, JUNB, MMP2, MMP9, NKX2_5, OVOL1, PDGFB, PTHLH, SERPINE1, SGK1, SKIL, SMAD4, SMAD5, SMAD6, SMAD7, SNAI1, SNAI2, TIMP1 and VEGFA, preferably CDC42EP3, GADD45A, GADD45B, HMGA2, ID1, IL11, INPP5D, JUNB, MMP2, MMP9, NKX2_5, OVOL1, PDGFB, PTHLH, SGK1, SKIL, SMAD4, SMAD5, SMAD6, TIMP1, VEGFA, more preferably CDC42EP3, GADD45A, GADD45B, ID1, JUNB, MMP9, PDGFB, SGK1, SKIL, SMAD5, SMAD6, TIMP1, VEGFA, even more preferably CDC42EP3, GADD45A, GADD45B, ID1, JUNB, MMP9, PDGFB, SGK1, SMAD5, TIMP1, VEGFA.

In a preferred embodiment, the AR pathway inhibitor is administered together with a TGFbeta pathway inhibitor, wherein the AR pathway inhibitor and the TGFbeta pathway inhibitor are the same compound or a different compound.

An AR pathway inhibitor may be Steroidal antiandrogens, Nonsteroidal antiandrogens, Androgen synthesis inhibitors, CYP17A1 inhibitors, CYP11A1 (P450scc) inhibitors, 5α-Reductase inhibitors and Antigonadotropins. Non-limiting examples are Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Darolutamide, Enzalutamide, Proxalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), Progestogens (e.g., chlormadinone acetate, cyproterone acetate, gestonorone caproate, medroxyprogesterone acetate, megestrol acetate), etanercept, resatorvid, filgotinib or combinations thereof.

Therefore in an embodiment, the AR inhibitor is selected from the group consisting of: Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Darolutamide, Enzalutamide, Proxalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), Progestogens (e.g., chlormadinone acetate, cyproterone acetate, gestonorone caproate, medroxyprogesterone acetate, megestrol acetate), etanercept, resatorvid, filgotinib or combinations thereof, preferably where in the AR inhibitor is selected from the group consisting of A-485, ARCC-4, ARD-266 Bicalutamide, etanercept, resatorvid, filgotinib or combinations thereof, more preferably wherein the AR inhibitor is A-485.

An TGFbeta pathway inhibitor may be Small molecule kinase inhibitors, Anti-TGF-0 ligand antibodies, Anti-TpR receptor antibodies or Antisense oligonucleotides. Non-limiting examples are TEW-7197, Galunisertib, LY2157299, Fresolimumab, XPA681, XPA089, LY2382770, LY3022859, ISTH0036, ISTH0047, Pyrrole-imidazole polyamides, etanercept, resatorvid, filgotinib or combinations thereof.

Therefore, in an embodiment the TGFbeta inhibitor is selected from the list comprising TEW-7197, Galunisertib, LY2157299, Fresolimumab, XPA681, XPA089, LY2382770, LY3022859, ISTH0036, ISTH0047, Pyrrole-imidazole polyamides, etanercept, resatorvid, filgotinib or combinations thereof.

Important for the invention is that activity of the AR, TGFbeta, MAPK-AP1, and JAK-STAT3 pathways is associated with immunosuppression. The finding that these pathways, especially the AR pathway, are on average abnormally activated in patients with sepsis, is important, because it indicates that a normal activity of this pathway, probably associated with a normal immune function. This appears to be substantiated by the finding of the inventors that the few patients with a normal AR pathway activity, similar to control subjects without infection, are the sepsis-survivors. This appears to suggest that normal immune function is required for survival of sepsis. Overall, it is clear that a good immune response is crucial for prevention of infection and for development of sepsis, and for prevention of sepsis-related mortality. It is again emphasized that it is surprising that this can be determined on the measured signaling pathway activities in whole blood.

The measurement of pathway activity on a blood sample, or on a specific subset of cells from a whole blood sample, provides information on the immune status of a patient with infection. It can be inferred that a patient with an infection, such as a urogenital infection associated with a bladder catheter, will be at higher risk to develop sepsis if the immune response is suppressed. Measuring activity of the AR pathway, and also the TGFbeta, MAPK-AP1, and JAK-STAT3 pathways in a blood sample of a patient with an infection will provide information on the risk for development of sepsis, which is high when activity of these pathways, especially the AR pathway, is increased. This will allow timely allow prediction of sepsis risk in patients with an infection, especially a bacterial infection.

For the first time the inventors demonstrate that determination of pathway activity can be used to diagnosis sepsis in a patient and stratify blood samples obtained from a subject to e.g. distinguish between sepsis and septic shock. More surprisingly however is the finding that this analysis can be performed on a blood sample such as a whole blood sample.

The present invention has been accomplished by intensively studying the activities of several signaling pathways such as the AR signaling pathway, the TGFbeta pathway, the MAPK-AP1 pathway and the JAK-STAT3 signaling pathway, in blood samples obtained from healthy control subjects, septic subjects that recovered and septic subjects that passed away as a result from septic shock.

Next it was evaluated whether the target genes for the identified relevant pathways can be used as a basis for making predictions (e.g. if a subject has sepsis or not). Using a simple model where based on gene expression levels corrected only for their correlation (up- or downregulated) it was found that a selection of three genes from either the AR cellular signaling pathway target genes, the TGFbeta cellular signaling pathway target genes, or the pooled AR and TGFbeta cellular signaling pathway target genes, suffice to make a prediction with very high specificity and a good sensitivity. Sensitivity of the prediction can be increased by increasing the amount of genes used in the assay or by more selectively selecting the genes (as demonstrated in Example 9), or by using the three (or more) genes in a pathway activity model as described herein.

The data presented in Example 9 demonstrate that the predictions described herein (e.g. diagnose a subject with sepsis, predict high or low mortality chance, predict risk of developing sepsis for a subject with a bacterial infection) may be made based on the expression levels of three or more genes from the lists descried herein. Therefore, the pathway models as described in more detail below may be used, however it is not required to do so.

The three or more genes as envisioned in the invention may be used as follows: the expression level of the three or more genes is determined based on mRNA levels in the sample (e.g. a blood sample obtained from a subject). The expression levels of the three or more genes are normalized, using one or more references, for example household genes. The normalized expression levels of three or more genes are multiplied with either “1” or “−1”, depending on their correlation with the pathway activity (+1 in case the gene expression is increased with higher pathway activity and −1 when the gene expression is decreased with higher pathway activity, the correlation of each gene is also indicated in Example 9). Next the normalized expression levels which have been corrected for expression correlation are added up or multiplied. The obtained value for the three or more expression levels can now be compared with a value obtained from the expression levels of three or more genes from a reference sample (e.g. a subject having sepsis or a healthy subject), or it can be compared with multiple references. Alternatively, the obtained value for the three or more expression levels can be compared with one or more set values. For example, based on reference samples obtained from healthy and septic subjects a cut-off value can be determined, which defines the upper limit of a non-septic subject and the lower limit of a septic subject. As a non-limiting example: based on the expression levels of three genes, in three healthy subjects the following values were calculated: 5, 8, 4; and in three septic subjects the following values were calculated 43, 30, 24. Based on these results a threshold value is calculated of 19. For a subject now the same three genes are used to calculate a score, where if the score is below 19 the subject is considered non-septic and if the score is above 19 the subject is considered septic. Therefore when the AR and/or TGFbeta pathway activity or the functional status of a blood sample are used in a method, prediction or diagnosis, as described herein, the AR and/or TGFbeta pathway activity or the functional status of a blood sample may be replaced by simply basing the method, prediction or diagnosis on three or more genes selected from the AR and/or TGFbeta pathway target genes.

The same principle can be applied on patients with an infection, particularly a bacterial infection, where an increased AR (and TGFbeta) signaling pathway activity correlates with an increased chance of developing sepsis. Three or more genes selected from the target genes of the AR and TGFbeta cellular signaling pathways as described herein can thus be used to predict or calculate the risk of a patient with an infection (e.g. a bacterial infection) to develop sepsis.

In example 9, the correlation between the different gene expression levels and pathway activity have further been used to provide a cut-off value for more selective lists of genes. These are indicated with the T values, where T=0 means not selection (all target genes are used) and T=0.3 refers to a cut-off value of a correlation of 0.3. The respective lists of genes for each T value (T=0, T=0.3, T=0.4, T=0.5) have been determined for the AR and TGFbeta cellular signaling pathways and are listed in Example 9. The genes listed in the embodiments and preferred embodiments for group 1 and 2 correspond to the selected target genes for the AR and TGFbeta pathways respectively and represent the different T values lists.

The ability to reliably determine whether a blood sample obtained from a subject or individual is obtained from a subject or individual with sepsis or not is desirable for several reasons. Currently a sepsis diagnosis is initially based on clinical parameters like respiratory rate, heart rate and blood pressure, which can be complemented by simple and not specific clinical chemistry lab measurements, such as lactate, CRP, electrolytes, urea, creatinine. This is not a very accurate diagnosis (not sufficiently sensitive and not specific), therefore when sepsis is suspected in a subject, the diagnosis needs to be confirmed by blood pathogen culture to detect the causative pathogen and profile its antibiotic resistance, which is a time consuming process which may take days to complete. In contrast, determining the functional status of a blood sample by determining one or more pathway activities based on extracted mRNA may be achieved on as little as 2-3 hours.

Once a sepsis diagnosis has been made, either by the methods described here or by other means, it is advantageous to determine whether a subject has a high or a low risk at progression to septic shock, and whether the patient has a high or low mortality risk. Currently, septic subjects, especially septic shock patients, are generally treated in intensive or urgent care units, which is costly. This may not always be necessary, considering the functional status of the blood sample as defined herein may be used to determine the risk at progression to septic shock and the mortality risk of a subject. Therefore once sepsis has been diagnosed, a risk assessment can be performed based on the functional status of the blood sample, for example based on the determined AR pathway activity, to determine whether the subject has a high or a low risk at progression to septic shock or high or low mortality risk. If a subject has low risk at septic shock or low mortality risk, subsequent treatment may not necessarily need to take place in the ICU and thus saving substantial cost. Furthermore, when a subject is determined to be at high risk, treatment in the ICU is beneficial, and additional monitoring or treatment may be warranted to further mitigate the risks, and for example a search for the causative pathogen may be intensified, and the source eradicated.

For the described reasons, the functional status of a blood sample is a useful tool. When used herein, “the functional status of a blood sample” is defined as the combined information of the determined activity or activities of the pathway or pathways from which the activity or activities have been determined. Generally, the activity of a pathway can be determined to be active or not active, or the activity can be determined in reference to control sample. A control sample may be a blood sample obtained from a healthy subject, but it may also refer to samples or data used to calibrate the model used to determine the pathway activity. Therefor a control sample is not necessarily a blood sample, but may also be a different sample with a known functional status of the pathway (i.e. active or not active). By comparing the activity to a reference such as a control sample, the activity may be expressed as a binary value (i.e. the pathway is active or not active), or it may be expressed as a relative value, represented by a number. Therefore, for example when only the AR pathway is determined on a blood sample, the functional status of the blood sample can be either qualified as active AR activity or inactive AR activity in reference to a control sample. Alternatively if the relative value of the pathway activity is represented by a number, and inactive control samples are defined as having a value of 0 and active control samples are defined as having a value of 1, the pathway activity as determined in the blood sample obtained from the subject may for example be 0.81, indicating the pathway activity is closer to active than inactive.

Therefore, the pathway activity based on the determined gene expression levels is preferably expressed as a numeric value. Using a model as described below, the gene expression levels of a pathway can be used to quantify the pathway activity with reference to the calibrated expression levels of the pathway gene and/or with reference to a control sample (e.g. a blood sample obtained from a healthy subject). This quantification can be a simple binary model (e.g. value 0 for pathway inactive, value 1 for pathway active), or can be more complex by quantifying the contribution of each gene from which the expression level has been determined, optionally multiplied by a weight factor. Therefore, “the status of a blood sample” as describe herein is the numeric value of the pathway activity as determined, or if multiple pathway activities are determined, it is the combined numeric values attributed to the determined pathways.

Therefore, preferably the status of the blood sample obtained from a subject comprises one or more activities of a signaling pathway, said signaling pathway activity is preferably: the AR signaling pathway activity, the AR and the TGFbeta signaling pathway activities, The AR and the MAPK-AP1 signaling pathway activities, the AR and the JAK-STAT3 signaling pathway activities, the AR, TGFbeta, and MAPK-AP1 signaling pathway activities, the AR, TGFbeta and JAK-STAT3 signaling pathway activities, the AR, MAPK-AP1 and JAK-STAT3 signaling activities and/or the AR, TGFbeta, MAPK-AP1 and JAK-STAT3 signaling activities, wherein said signaling activities are based on the determined expression levels of three or more target genes of the respective pathways.

In accordance with this, it is an embodiment of the present invention that the determining of the functional status of a blood sample is further based on a respective reference signaling pathway activity or combination of reference activities of signaling pathways. Similarly, the determining of the diagnosis or mortality risk may be further based on a reference activity of the respective signaling pathway. A reference activity reflects activity of the respective signaling pathway found in blood samples obtained from healthy subjects and obtained from septic patients with known clinical outcomes (e.g. septic shock recovered, septic shock died).

For the purpose of the invention determining the expression levels of the genes or target genes based on the extracted RNA may be a part of the method, meaning the method includes the step of determining the expression levels of the genes or target genes on RNA extracted from the blood sample obtained from the patient using methods known to the skilled person or described herein. The method may further include the step of obtaining a blood sample form the patient in order to extract the RNA. Alternatively, the expression levels may have been determined separately and the demining step (of the expression levels of the target genes) is not an active step in the method of the invention. In such case the expression levels are provided as an input value, e.g. a relative expression level in reference to one or more control gene expression levels.

By comparing each of the three or more gene expression levels with three or more reference expression levels, in the subject to be diagnosed, a prediction can be made about the state of the subject (e.g. septic or non-septic, likelihood to develop sepsis, likelihood of mortality from sepsis). Alternatively, by comparing each of the reference pathway activities to each of the respective pathway activities in the subject to be diagnosed, the status of the blood sample comprising each of the respective pathway activities can be determined.

When used herein, “expression level” refers to quantifying the number of mRNA copies transcribed from a gene. Generally, this number will not be an absolute value but a relative value, and therefore is preferably normalized for example in reference to the expression of one or more housekeeping genes. Housekeeping genes are genes which are assumed to have constant expression levels independent of cell type and/or functional status of the cell (i.e. from a diseased or healthy subject), and therefore can be used to normalize experimentally determined relative expression levels. Housekeeping genes are generally known to the skilled person, non-limiting examples of housekeeping genes that may be used for normalization are beta-actin, glyceraldehyde-3-phosphate dehydrogenase (GAPDH) and Transcription factor IID TATA binding protein (TBP).

Sets of cellular signaling pathway target genes whose expression levels are preferably analyzed have been identified, alternatively methods for identifying suitable target genes are described herein. For use to determine pathway activity, for example by a mathematical model, three or more, for example, three, four, five, six, seven, eight, nine, ten, eleven, twelve or more, target genes from each assessed cellular signaling pathway can be analyzed to determine pathway activities.

The blood sample obtained from a subject can be any type of blood sample, that is, the blood may be drawn for example using a cannula and may be whole blood or a defined fraction of blood, such as isolated PBMCs, isolated CD4+ cells, mixed CD8+ and T cells, isolated neutrophils or isolated monocytes. Preferably the sample is whole blood. The presented invention is based on the surprising finding that whole blood, despite the versatility of cells comprised therein, can be used for pathway analysis to determine the functional status of said blood sample, and wherein said functional status of said blood sample can be used for, for example, diagnosis and prognosis of the subject having sepsis or suspected to have sepsis from which the blood sample has been obtained.

The activity of one or more signaling pathways can thus be used as a biomarker that characterizes the functional status of a blood sample, which will be useful for early prediction of the development of sepsis in a patient with an infection, diagnosis of sepsis in subject, predicting the progression to septic shock and mortality risk of a subject with sepsis, and choice of therapy for a subject with sepsis.

In an embodiment the signaling pathway measurements are performed using qPCR, multiple qPCR, multiplexed qPCR, ddPCR, RNAseq, RNA expression array or mass spectrometry. For example, a gene expression microarray data, e.g. Affymetrix microarray, or RNA sequencing methods, like an Illumina sequencer, can be used.

The term “subject”, as used herein refers to any living being. In some embodiments, the subject is an animal, preferably a mammal. In certain embodiments, the subject is a human being, such as a medical subject. Although the invention is not necessarily limited to a particular group of subjects, it will be apparent that a subject having sepsis or a subject suspected to have sepsis or a subject at risk for developing sepsis profits the most form the invention described herein. It is therefore preferred that the subject from which the blood sample has been obtained is a subject having sepsis, in case sepsis has already been confirmed be alternative means (e.g. a blood culture or by the claimed method), or is a subject suspected to have sepsis or an subject at risk of developing sepsis. A subject suspected to have sepsis may be a subject which meets one or more criteria that define SIRS or sepsis, such presence of fever, tachycardia, tachypnea, and/or leukocytosis or leukopenia. Alternatively a subject suspected to have sepsis may be a subject at risk of having or developing sepsis, e.g. a subject having an infection that may lead to sepsis or a subject having, cancer, diabetes, reduced immunity, a subject having spent prolonged time in the intensive care unit, subject who are born preterm, have a low AGPAR score, etc. The subject may also be a subject at risk of developing sepsis, when used herein a “subject at risk of developing sepsis” refers to a subject which does not currently have sepsis but has one or more increased risk factors which may lead the subject to develop sepsis, for example the presence of urinary catheters, open wounds or wounds with drains, intravascular lines etc.

When used herein, the term “clinical parameter” refers to respiratory rate, heart rate, blood pressure. The term “clinical parameter” may further refer to a symptom selected from: fever, chills, very low body temperature, peeing less than usual, nausea, vomiting, diarrhea, fatigue, weakness, blotchy or discolored skin, sweating, clammy skin or severe pain.

The blood sample to be used in accordance with the present invention can be an extracted sample, that is, a sample that has been extracted from the subject. Examples of the sample include, but are not limited to whole blood sample, isolated PBMCs, isolated CD4+ cells, mixed CD8+ and T cells, isolated neutrophils or isolated monocytes. Isolated PBMCs, isolated CD4+ cells, mixed CD8+ and T cells, isolated neutrophils or isolated monocytes are generally obtained from whole blood samples be methods known to the skilled person. Further the skilled person is familiar with how to obtain a whole blood sample from the subject using conventional methods to draw blood. The term “sample”, as used herein, also encompasses the case where e.g. cells, tissue and/or body fluid have been taken from the subject and, e.g., have been put on a microscope slide or fixative, and where for performing the claimed method a portion of this sample is extracted, e.g., by means of Laser Capture Microdissection (LCM), or by punching, or by scraping off the cells of interest from the slide, or by fluorescence-activated cell sorting techniques. In addition, the term “sample”, as used herein, also encompasses the case where e.g. cells, tissue and/or body fluid have been taken from the subject and have been put on a microscope slide, and the claimed method is performed on the slide. Preferably the sample is a body fluid, particularly whole blood, or one or more cell types isolated from a whole blood sample.

The terms “pathway”, “signal transduction pathway”, “signaling pathway” and “cellular signaling pathway” are used interchangeably herein.

An “activity of a signaling pathway” may refer to the activity of a signaling pathway associated transcription factor (TF) element in the sample, the TF element controlling transcription of target genes, in driving the target genes to expression, i.e., the speed by which the target genes are transcribed, e.g. in terms of high activity (i.e. high speed) or low activity (i.e. low speed), or other dimensions, such as levels, values or the like related to such activity (e.g. speed). Accordingly, for the purposes of the present invention, the term “activity”, as used herein, is also meant to refer to an activity level that may be obtained as an intermediate result during “pathway analysis” as described herein.

The term “transcription factor element” (TF element), as used herein, preferably refers to an intermediate or precursor protein or protein complex of the active transcription factor, or an active transcription factor protein or protein complex which controls the specified target gene expression. For example, the protein complex may contain at least the intracellular domain of one of the respective signaling pathway proteins, with one or more co-factors, thereby controlling transcription of target genes. Preferably, the term refers to either a protein or protein complex transcriptional factor triggered by the cleavage of one of the respective signaling pathway proteins resulting in an intracellular domain.

The term “target gene”, as used herein, means a gene whose transcription is directly or indirectly controlled by a respective transcription factor element. The “target gene” may be a “direct target gene” and/or an “indirect target gene” (as described herein).

Pathway analysis enables quantitative measurement of signal transduction pathway activity in blood cells, based on inferring activity of a signal transduction pathway from measurements of mRNA levels of the well-validated direct target genes of the transcription factor associated with the respective signaling pathway (see for example W Verhaegh et al., 2014, supra; W Verhaegh, A van de Stolpe, Oncotarget, 2014, 5(14):5196).

In accordance with this, it is an embodiment of the present invention that the determining of the functional status of a blood sample and/or its subsequent uses such as diagnosing a patient or predicting a mortality risk is further based on a respective combination of reference activities of signaling pathways. Similarly, the determining of the signaling pathway abnormality factor may be further based on a reference activity of the respective signaling pathway. A reference activity reflects activity of the respective signaling pathway found in a blood sample of healthy subjects.

By comparing each of the reference pathway activities to each of the respective pathway activities in the subject to be diagnosed, a functional status of the blood sample comprising each of the respective pathways can be determined. The functional status of the blood sample indicates whether the activity of the respective pathway(s) deviates (abnormally) from the reference activity of the respective pathway(s). The functional status of the blood sample may then be translated into diagnosis or a mortality risk. The functional status of a blood sample may also be computed directly from the combination of pathway activities. The functional status of a blood sample can be considered as multi-pathway score, MPS, and denotes a likelihood that a subject has sepsis, or the risk of mortality as a result of sepsis. Accordingly, the “the functional status of a blood sample”, refers to a dimension, e.g. a level or a value, relating the combination of pathway activities to a likelihood that the subject has sepsis, or the risk of mortality as a result of sepsis.

The term “sepsis” as used herein refers to a condition that arises when the body's response to infection causes injury to its tissues and organs. Sepsis is an inflammatory immune response triggered by an infection. Bacterial infections are the most common cause, but fungal, viral, and protozoan infections can also lead to sepsis. Common locations for the primary infection include the lungs, brain, urinary tract, skin, and abdominal organs.

The functional status of the blood sample is based on a single cellular signaling pathway activity or on a “combination of activities of cellular signaling pathways”. This means that the functional status of the blood sample is influenced by the activities of one or more cellular signaling pathways. The activities of the one or more cellular signaling pathways can be inferred and/or combined by a mathematical model as described herein. In a preferred embodiment, the functional status of the blood sample is based on a combination of signaling pathway activities comprising activities of more than 2 cellular signaling pathways. Such combination of signaling pathway activities may include the activities of 3 or 4, or even more than 4 such as 5, 6, 7 or 8, or even more, different signaling pathways.

In general, many different formulas can be devised for determining a functional status of the blood sample that is based on a combination of activities of two or more cellular signaling pathways in a subject, i.e.:

$$MPS = F ( Pi ) + X , with i = 1 … N ,$$

wherein MPS denotes the functional status of the blood sample and/or risk score (the term “MPS” is used herein as an abbreviation for “Multi-Pathway Score” in order to denote that the functional status of the blood sample can be influenced by the activities of two or more cellular signaling pathways), Pi denotes the activity of cellular signaling pathway i, N denotes the total number of cellular signaling pathways used for calculating the functional status of the blood sample, and X is a placeholder for possible further factors and/or parameters that may go into the equation. Such a formula may be more specifically a polynomial of a certain degree in the given variables, or a linear combination of the variables. The weighting coefficients and powers in such a polynomial may be set based on expert knowledge, but typically a training data set with known ground truth, e.g., survival data, is used to obtain estimates for the weighting coefficients and powers of the formula above. The activities may be combined using the formula above and will subsequently generate an MPS. Next, the weighting coefficients and powers of the scoring function may be optimized such that a high MPS correlates with a higher probability that the patient has sepsis and/or has a high mortality risk, and vice versa. Optimizing the scoring function's correlation with known data can be done using a multitude of analysis techniques, e.g., a Cox proportional hazards test (as preferably used herein), a log-rank test, a Kaplan-Meier estimator in conjunction with standard optimization techniques, such as gradient-descent or manual adaptation, and so on.

When used herein, the term “risk score” or “risk factor” general refers to a prediction, risk assessment or diagnosis for a subject based on the functional status of the blood sample. For example the risk score may be the diagnosis of a subject to have sepsis (the risk that the subject is septic), the mortality risk of a septic subject, and/or the risk of developing sepsis for a non-septic subject, the risk of relapsing of a subject which was previously diagnosed with sepsis.

Preferably the determining of the activity or activities of the signaling pathway(s), the combination of multiple pathway activities and applications thereof is performed as described for example in the following documents, each of which is hereby incorporated in its entirety for the purposes of determining activity of the respective signaling pathway: published international patent applications WO2013011479 (titled “ASSESSMENT OF CELLULAR SIGNALING PATHWAY ACTIVITY USING PROBABILISTIC MODELING OF TARGET GENE EXPRESSION”), WO2014102668 (titled “ASSESSMENT OF CELLULAR SIGNALING PATHWAY ACTIVITY USING LINEAR COMBINATION(S) OF TARGET GENE EXPRESSIONS”), WO2015101635 (titled “ASSESSMENT OF THE PI3K CELLULAR SIGNALING PATHWAY ACTIVITY USING MATHEMATICAL MODELLING OF TARGET GENE EXPRESSION”), WO2016062891 (titled “ASSESSMENT OF TGF-P CELLULAR SIGNALING PATHWAY ACTIVITY USING MATHEMATICAL MODELLING OF TARGET GENE EXPRESSION”), WO2017029215 (titled “ASSESSMENT OF NFKB CELLULAR SIGNALING PATHWAY ACTIVITY USING MATHEMATICAL MODELLING OF TARGET GENE EXPRESSION”), WO2014174003 (titled “MEDICAL PROGNOSIS AND PREDICTION OF TREATMENT RESPONSE USING MULTIPLE CELLULAR SIGNALLING PATHWAY ACTIVITIES”), WO2016062892 (titled “MEDICAL PROGNOSIS AND PREDICTION OF TREATMENT RESPONSE USING MULTIPLE CELLULAR SIGNALING PATHWAY ACTIVITIES”), WO2016062893 (titled “MEDICAL PROGNOSIS AND PREDICTION OF TREATMENT RESPONSE USING MULTIPLE CELLULAR SIGNALING PATHWAY ACTIVITIES”), WO2018096076 (titled “Method to distinguish tumor suppressive FOXO activity from oxidative stress”), and in the patent applications WO2018096076 (titled “Method to distinguish tumor suppressive FOXO activity from oxidative stress”), WO2019068585 (titled “Assessment of Notch cellular signaling pathway activity using mathematical modelling of target gene expression”), WO2019120658 (titled “Assessment of MAPK-MAPK-AP1 cellular signaling pathway activity using mathematical modelling of target gene expression”), WO2019068543 (titled “Assessment of JAK-JAK-STAT3 cellular signaling pathway activity using mathematical modelling of target gene expression”), WO2019068562 (titled “Assessment of JAK-STAT1/2 cellular signaling pathway activity using mathematical modelling of target gene expression”), and WO2019068623 (titled “Determining functional status of immune cells types and immune response”).

The models have been biologically validated for ER, AR, PI3K-FOXO, HH, Notch, TGF-β, Wnt, NFkB, JAK-STAT1/2, JAK-JAK-STAT3 and MAPK-MAPK-AP1 pathways on several cell types.

Unique sets of cellular signaling pathway target genes whose expression levels are preferably analyzed have been identified. For use in the mathematical models, three or more, for example, three, four, five, six, seven, eight, nine, ten, eleven, twelve or more, target genes from each assessed cellular signaling pathway can be analyzed to determine pathway activities.

Common to the pathway analysis methods for determining the activities of the different signaling pathways as disclosed herein is a concept, which is preferably applied herein for the purposes of the present invention, wherein the activity of a signaling pathway in a cell such as a cell present in a blood sample is determinable by receiving expression levels of one or more, preferably three or more, target genes of the signaling pathway, determining an activity level of a signaling pathway associated transcription factor (TF) element in the sample, the TF element controlling transcription of the three or more target genes, the determining being based on evaluating a calibrated mathematical pathway model relating expression levels of the one or more, preferably three or more target genes to the activity level of the signaling pathway, and optionally inferring the activity of the signaling pathway in the cell present in a blood sample based on the determined activity level of the signaling pathway associated TF element. As described herein, the activity level can be directly used as an input to determine functional status of the blood sample and/or diagnosis and/or risk score, which is also contemplated by the present invention.

The term “activity level” of a TF element, as used herein, denotes the level of activity of the TF element regarding transcription of its target genes.

The calibrated mathematical pathway model may be a probabilistic model, preferably a Bayesian network model, based on conditional probabilities relating the activity level of the signaling pathway associated TF element and the expression levels of the three or more target genes, or the calibrated mathematical pathway model may be based on one or more linear combination(s) of the expression levels of the three or more target genes. For the purposes of the present invention, the calibrated mathematical pathway model is preferably a centroid or a linear model, or a Bayesian network model based on conditional probabilities.

In particular, the determination of the expression level and optionally the inferring of the activity of a signaling pathway in the subject may be performed, for example, by inter alia (i) evaluating a portion of a calibrated probabilistic pathway model, preferably a Bayesian network, representing the cellular signaling pathways for a set of inputs including the expression levels of the three or more target genes of the cellular signaling pathway measured in a sample of the subject, (ii) estimating an activity level in the subject of a signaling pathway associated transcription factor (TF) element, the signaling pathway associated TF element controlling transcription of the three or more target genes of the cellular signaling pathway, the estimating being based on conditional probabilities relating the activity level of the signaling pathway associated TF element and the expression levels of the three or more target genes of the cellular signaling pathway measured in the sample of the subject, and optionally (iii) inferring the activity of the cellular signaling pathway based on the estimated activity level of the signaling pathway associated TF element in the sample of the subject. This is described in detail in the published international patent application WO 2013/011479 A2 (“Assessment of cellular signaling pathway activity using probabilistic modeling of target gene expression”), the contents of which are herewith incorporated in their entirety.

In an exemplary alternative, the determination of the expression level and optionally the inferring of the activity of a cellular signaling pathway in the subject may be performed by inter alia (i) determining an activity level of a signaling pathway associated transcription factor (TF) element in the sample of the subject, the signaling pathway associated TF element controlling transcription of the three or more target genes of the cellular signaling pathway, the determining being based on evaluating a calibrated mathematical pathway model relating expression levels of the three or more target genes of the cellular signaling pathway to the activity level of the signaling pathway associated TF element, the mathematical pathway model being based on one or more linear combination(s) of expression levels of the three or more target genes, and optionally (ii) inferring the activity of the cellular signaling pathway in the subject based on the determined activity level of the signaling pathway associated TF element in the sample of the subject. This is described in detail in the published international patent application WO 2014/102668 A2 (“Assessment of cellular signaling pathway activity using linear combination(s) of target gene expressions”).

Further details regarding the inferring of cellular signaling pathway activity using mathematical modeling of target gene expression can be found in W Verhaegh et al., 2014, supra.

To facilitate rapid identification of references, the above-mentioned references have been assigned to each signaling pathway of interest here and exemplarily corresponding target genes suitable for determination of the signaling pathway's activity have been indicated. In this respect, particular reference is also made to the sequence listings for the target genes provided with the above-mentioned references.

AR: KLK2, PMEPA1, TMPRSS2, NKX3_1, ABCC4, KLK3, FKBP5, ELL2, UGT2B15, DHCR24, PPAP2A, NDRG1, LRIG1, CREB3L4, LCP1, GUCY1A3, AR and EAF2 (WO 2013/011479, WO 2014/102668); KLK2, PMEPA1, TMPRSS2, NKX3_1, ABCC4, KLK3, FKBP5, ELL2, UGT2B15, DHCR24, PPAP2A, NDRG1, LRIG1, CREB3L4, LCP1, GUCY1A3, AR, and EAF2 (WO 2014/174003); TGF-P: ANGPTL4, CDC42EP3, CDKNIA, CDKN2B, CTGF, GADD45A, GADD45B, HMGA2, ID1, IL 11, SERPINE1, INPP5D, JUNB, MMP2, MMP9, NKX2-5, OVOL1, PDGFB, PTHLH, SGK1, SKIL, SMAD4, SMAD5, SMAD6, SMAD7, SNAI1, SNAI2, TIMP1 and VEGFA (WO 2016/062891, WO 2016/062893); MAPK-AP-1: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53 and VIM (WO 2019/120658); and JAK-JAK-STAT3: AKT1, BCL2, BCL2L1, BIRC5, CCND1, CD274, CDKN1A, CRP, FGF2, FOS, FSCN1, FSCN2, FSCN3, HIF1A, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSFlB, TWIST1, VIM and ZEB1 (WO 2019/068543).

In an embodiment the signaling pathway measurements are performed using qPCR, multiple qPCR, multiplexed qPCR, ddPCR, RNAseq, RNA expression array or mass spectrometry. For example, gene expression microarray data, e.g. Affymetrix microarray, or RNA sequencing methods, like an Illumina sequencer, can be used.

The calibrated mathematical pathway model is preferably a centroid or a linear model, or a Bayesian network model based on conditional probabilities. For example, the calibrated mathematical pathway model may be a probabilistic model, preferably a Bayesian network model, based on conditional probabilities relating the functional status of the blood sample and/or the risk score and the activities of the signaling pathways, or the calibrated mathematical pathway model may be based on one or more linear combination(s) of the activities of the signaling pathways.

Unique sets of cellular signaling pathway target genes whose expression levels are preferably analyzed have been identified. For use in the mathematical models, three or more, for example, three, four, five, six, seven, eight, nine, ten, eleven, twelve or more, target genes from each assessed cellular signaling pathway can be analyzed to determine pathway activities.

As a non-limiting example, the following method can be used to generate a model for determining signaling pathway activity: in a plurality of datasets expression RNA levels of different genes are determined in samples where the pathway is assumed to be active and samples where the pathway is assumed to be not active. The expression levels are normalized, for example based on the expression levels of house-keeping genes. Based on the normalized expression levels of the samples where the pathway is assumed to be active or assumed to be inactive, a threshold can be determined, where if the normalized expression level of a gene in a sample is below the threshold the pathway is more likely to inactive and if the expression level is above the threshold the pathway is more likely to be active. Based on this threshold a simple model can be constructed, where a value is assigned to the expression level, as determined in a blood sample of a subject with sepsis or suspected to have sepsis, and the pathway activity is determined as the sum of these values for each gene for which the expression level is determined. Alternatively, the values obtained for each gene for the respective pathway can be compared with values obtained for said gene in a reference blood sample from a healthy subject (i.e. a subject not having sepsis).

According to an embodiment of the present invention
said determining the expression level of three or more target genes of the AR signaling pathway, the TGFbeta signaling pathway, the MAPK-AP1 signaling pathway and/or the JAK-STAT3 signaling pathway comprises:
determining the expression level of three or more target genes, for example 3, 4, 5, 6, 7, 8, 9, 10, 11, 12 or more target genes of the AR signaling pathway selected from the list consisting of: KLK2, PMEPA1, TMPRSS2, NKX3_1, ABCC4, KLK3, FKBP5, ELL2, UGT2B15, DHCR24, PPAP2A, NDRG1, LRIG1, CREB3L4, LCP1, GUCY1A3, AR and EAF2, preferably wherein the set of target genes of the AR pathway comprises three or more target genes, for example 3, 4, 5, 6, 7, 8, 9, 10, 11 or 12 target genes selected from the group consisting of ELL2, FKBP5, GUCY1A3, LRIG1, PLAU, PMEPA1, PRKACB, SGK1, NDRG1, CREB3L4, DHCR24 or PTPN1, and/or;
determining the expression level of three or more target genes, for example 3, 4, 5, 6, 7, 8, 9, 10, 11, 12 or more target genes of the TGFbeta signaling pathway comprises determining the expression level of three or more target genes selected from the list consisting of ANGPTL4, CDC42EP3, CDKN1A, CTGF, GADD45A, GADD45B, HMGA2, ID1, IL11, JUNB, PDGFB, PTHLH, SERPINE1, SGK1, SKIL, SMAD4, SMAD5, SMAD6, SMAD7, SNAI2, VEGFA, preferably wherein the set of target genes of the TGFbeta pathway comprises three or more target genes, for example 3, 4, 5, 6, 7, 8, 9, 10, 11 or 12 target genes selected from the group consisting of CDC42EP3, GADD45A, ID1, MMP9, SGK1, SMAD5, SMAD7, VEGFA, JUNB, TIMP1, SKIL and CCKN1A, and/or;
determining the expression level of three or more target genes of the MAPK-AP1 signaling pathway comprises determining the expression level of three or more target genes, for example 3, 4, 5, 6, 7, 8, 9, 10, 11, 12 or more target genes selected from the list consisting of BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM, preferably wherein the set of target genes of the MAPK-AP1 pathway comprises three or more target genes, for example 3, 4, 5, 6, 7, 8, 9, 10, or 11 target genes selected from the group consisting of DNMT1, EGFR, ENPP2, GLRX, MMP9, PLAUR, TIMP1, LOR, EZR, DDIT3 and TP53, and/or;
determining the expression level of three or more target genes, for example 3, 4, 5, 6, 7, 8, 9, 10, 11, 12 or more target genes of the JAK-STAT3 signaling pathway comprises determining the expression level of three or more target genes selected from the list consisting of AKT1, BCL2, BCL2L1, BIRC5, CCND1, CD274, CDKN1A, CRP, FGF2, FOS, FSCN1, FSCN2, FSCN3, HIF1A, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSFlB, TWIST1, VIM, and ZEB1 preferably wherein the set of target genes of the JAK-STAT3 pathway comprises three or more target genes, for example 3, 4, 5, 6, 7, 8, 9, 10, 11, 12 or more target genes selected from the group consisting of BCL2, BIRC5, CD274, FOS, HSPA1A, JUNB, MMP9, STAT1, TIMP1, BCL2L1, HSPA1B, HSP90AB1, HSP90B1, POU2F1 and ICAM1.

Therefore, in an embodiment the method according to the invention comprises the step of diagnosing the subject from which the blood sample has been obtained, wherein said subject is diagnosed to have sepsis or wherein said subject is diagnosed to not have sepsis based on:
a clinical parameter, and
the functional status of the blood sample,
the method further comprising comparing the functional status of the blood sample of the subject to at least one functional status of a blood sample obtained from a healthy or non-septic control subject. In a preferred embodiment the subject is diagnosed to have sepsis if the functional status of the blood sample comprises an AR signaling pathway activity which AR signaling pathway activity is determined to be higher than the AR signaling pathway activity determined in the control blood sample obtained from a healthy or non-septic control and the subject from which the blood sample has been obtained has at least one clinical parameter associated with sepsis. It was found by the inventors that the pathway activity determined on a blood sample obtained from a subject can be used to diagnoses said subject to have sepsis or not. As detailed in the experimental data, determining the AR signaling pathway is sufficient to distinguish blood samples obtained from subjects having sepsis and blood samples obtained from healthy individuals. Optionally other pathway activities can be included in the diagnosis, such as the TGFbeta signaling pathway activity, the MAPK-AP1 signaling pathway activity, and/or the JAK-STAT3 signaling pathway activity. Therefore, the functional status of a blood sample as determined herein can be used as a diagnostic tool to quickly diagnose a subject.

Based on the expression level in a blood sample of three or more target genes from the AR pathway, the AR signaling pathway activity, and thus the functional status of the blood sample can be determined. This AR signaling pathway can be expressed as a quantitative value and compared with the AR signaling pathway activity determined on the blood samples obtained from either healthy subjects or known septic subjects. Therefore, the step of diagnosing a subject preferably further includes comparing the functional status of the blood sample of the subject with the functional status of a blood sample obtained from known septic patients. Because the diagnosing step is based on the functional status of the blood sample, this step optionally further uses the determined TGFbeta, MAPK-AP1, and/or JAK-STAT3 signaling pathway activities if they are determined. By including additional pathways, the certainty of the diagnosis may be further improved.

The functional status of a blood sample comprises at least the AR signaling pathway activity expressed as a numeric value. Thus by comparing the functional status of a blood sample from a subject, with the functional status of a control blood sample obtained from a healthy subject a diagnosis can be made based on the difference or similarity of the numeric values represented by at least the AR signaling pathway activities. Therefore, said comparing preferably is done using a plurality of functional statuses of reference blood samples to increase accuracy. More preferably said comparing further includes comparing with one or more, preferably a plurality, additional reference functional statuses of blood samples obtained from known septic subjects.

For example, when using multiple reference blood samples of healthy individuals, the AR signaling pathway activities can be calculated for each sample, and an average value can be determined. The AR signaling pathway activity of the subject to be diagnosed can be similar to the average reference AR signaling pathway activity (e.g. within 1 or within 2 standard deviations of the calculated average activity), in which case the subject is diagnosed to not have sepsis. Alternatively, the AR signaling pathway can be higher compared to the calculated average AR signaling pathway activity (e.g. at least 1 or 2 standard deviation higher than the average value), in which case the subject is diagnosed to have sepsis. Optionally the other pathway activities can be included in this comparison. The above method can be used regardless of the method used to calculate the signaling pathway activity, provided the same method is used for sample from the subject to be diagnosed and the reference samples.

Preferably for the purpose of diagnosing a subject with sepsis, determining the expression level of three or more target genes of the AR signaling pathway, the TGFbeta signaling pathway, the MAPK-AP1 signaling pathway and/or the JAK-STAT3 signaling pathway comprises:
determining the expression level of three or more target genes, for example 3, 4, 5, 6, 7, 8, 9, 10, 11, 12 or more target genes of the AR signaling pathway wherein three or more target genes, e.g. 3, 4 or 5, are selected form the group consisting of: FKBP5, LRIG1, PMEPA1, DHCR24 and LCP1 and/or;
determining the expression level of three or more target genes, for example 3, 4, 5, 6, 7, 8, 9, 10, 11, 12 or more target genes of the TGFbeta signaling pathway wherein three or more target genes, e.g. 3, 4 or 5, are selected form the group consisting of: MMP9, GADD45A, CDC42EP3, TIMP1 and SMAD5, and/or;
determining the expression level of three or more target genes of the MAPK-AP1 signaling pathway comprises determining the expression level of three or more target genes, for example 3, 4, 5, 6, 7, 8, 9, 10, 11, 12 or more target genes of the MAPK-AP1 pathway wherein three or more target genes, e.g. 3, 4 or 5, are selected form the group consisting of: MMP9, TIMP1, DNMT1, FASLG and PLAUR, and/or;
determining the expression level of three or more target genes, for example 3, 4, 5, 6, 7, 8, 9, 10, 11, 12 or more target genes of the JAK-STAT3 signaling pathway wherein three or more target genes, e.g. 3, 4 or 5, are selected form the group consisting of: MMP9, BCL2, TIMP2, HSPA1A and HSPA1AB.

Therefore, the invention further relates to a method for diagnosing a subject, based on RNA extracted from a blood sample obtained from the subject, the method comprising the steps of:
determining or receiving the result of a determination of the expression level of three or more target genes of the AR pathway;
determining the AR signaling pathway activity, based on the determined expression levels of said three or more target genes of the AR signaling pathway;
and optionally:
determining or receiving the result of a determination of the expression level of three or more target genes of the TGFbeta pathway;
determining the TGFbeta signaling pathway activity, based on the determined expression levels of said three or more target genes of the TGFbeta signaling pathway;
determining or receiving the result of a determination of the expression level of three or more target genes of the MAPK-AP1 pathway;
determining the MAPK-AP1 signaling pathway activity, based on the determined expression levels of said three or more target genes of the MAPK-AP1 signaling pathway;
determining or receiving the result of a determination of the expression level of three or more target genes of the JAK-STAT3 pathway;
determining the JAK-STAT3 signaling pathway activity, based on the determined expression levels of said three or more target genes of the JAK-STAT3 signaling pathway;
wherein said method further comprises the step of diagnosing the subject from which the blood sample has been obtained, wherein said subject is diagnosed to have sepsis or wherein said subject is diagnosed to not have sepsis based on:
a clinical parameter, and
the activity of the determined signaling pathways in the blood sample,
the method further comprising comparing the activity of the determined signaling pathways in the blood sample of the subject to at the activity of the determined signaling pathways in at least one blood sample obtained from a healthy or non-septic control subject,
wherein said blood sample is obtained from a subject with sepsis or obtained from a subject suspected to have sepsis or a subject at risk of developing sepsis. In a preferred embodiment the subject is diagnosed to have sepsis if AR signaling pathway activity which AR signaling pathway activity is determined to be higher than the AR signaling pathway activity determined in the control blood sample obtained from a healthy or non-septic control and the subject from which the blood sample has been obtained has at least one clinical parameter associated with sepsis.

Therefore, in an embodiment of the invention the subject is diagnosed to have sepsis if the functional status of the blood sample comprises an AR signaling pathway activity which AR signaling pathway activity is determined to be higher than the AR signaling pathway activity determined in the reference blood sample obtained from a healthy subject. Alternatively, the diagnosis can be based on the expression levels of the three or more genes directly. Alternatively, the comparison can further include reference blood samples obtained from known septic subjects. In such case the expression levels of the three or more genes or the functional status of the blood sample of the subject can be compared with both the expression levels of three or more genes or the functional status of blood samples of healthy individuals and the expression levels of three or more genes or the functional status of blood samples of known septic subjects. In such case the numeric values assigned to the AR signaling pathway activity (and other pathway activities, if determined) can be compared. For example when the subject has an AR signaling pathway which is close to the average value for known septic subjects, e.g. within 1 or 2 standard deviations, the subject is diagnosed with sepsis, or when the value is close to the average value for healthy subjects, e.g. within 1 or 2 standard deviations, the subject is diagnosed to be non-septic. Alternatively, a statistical method can be used to determine whether the subject is more likely to be in the non-septic or septic group (meaning, the numeric value assigned to the AR signaling pathway is closer to the normal (healthy) average or closer to the septic average value). Other signaling pathway activities can optionally be included in this calculation. When using multiple signaling pathway activities for the diagnosis step, e.g. both AR signaling pathway activity and TGFbeta signaling pathway activity, a clustering method may be used to cluster the healthy control subjects, the known septic subject and the subject to be diagnosed, based on the pathway activities, in order to establish whether the subject to be diagnosed falls within the healthy or the septic group. When used herein, reference blood sample form known septic subjects are blood sample obtained from subjects in which later the diagnosis sepsis has been confirmed, for example by blood culture.

In an embodiment of the method according to the invention, said expression levels of the three or more genes are used in predicting the mortality risk for the subject from which the blood sample has been obtained,
wherein said prediction is based on a comparison of the expression levels of the three or more genes of the subject with a plurality of reference expression levels of three or more genes obtained from reference subjects, wherein said plurality of reference expression levels of the three or more genes obtained from reference subjects comprises expression levels of the three or more genes obtained from subject with sepsis which is a non-survivor and expression levels of the three or more genes obtained from subject with sepsis which is a survivor, and optionally further comprises expression levels of the three or more genes obtained from a healthy or non-septic control subject,
wherein the subject from which the blood sample is obtained is confirmed to have sepsis, and
wherein a low mortality risk is predicted when the expression levels of the three or more genes obtained from the subject with sepsis are similar to expression levels of the three or more genes obtained from reference subject with sepsis which is a survivor or when the expression levels of the three or more genes obtained from the subject with sepsis are similar to the expression levels of the three or more genes obtained from the at least one healthy or non-septic control subject, and
wherein a high mortality risk is predicted when the expression levels of the three or more genes obtained from the subject with sepsis are similar to the expression levels of the three or more genes obtained from the reference subject with sepsis which is a non-survivor.

In an alternative embodiment of the invention, the functional status of the blood sample is used in predicting the mortality risk for the subject from which the blood sample has been obtained,
wherein said prediction is based on a comparison of the functional status of the blood sample of the subject with a plurality of reference functional statuses of the blood samples obtained from reference subjects, wherein said plurality of reference functional statuses of the blood samples obtained from reference subjects comprises at least one functional status of blood sample obtained from subject with sepsis which is a non-survivor and at least one functional status of blood samples obtained from subject with sepsis which is a survivor, and optionally further comprises at least one functional status of blood samples obtained from a healthy or non-septic control subject,
wherein the subject from which the blood sample is obtained is confirmed to have sepsis, and
wherein a low mortality risk is predicted when the functional status of the blood sample obtained from the subject with sepsis is similar to the at least one functional status of the blood sample obtained from reference subject with sepsis which is a survivor or when the functional status of the blood sample obtained from the subject with sepsis is similar to the at least one functional status of the blood sample obtained from the at least one healthy or non-septic control subject, and
wherein a high mortality risk is predicted when the functional status of the blood sample obtained from the subject with sepsis is similar to the at least one functional status of the blood sample obtained from the reference subject with sepsis which is a non-survivor.

In an embodiment said comparing of the three or more genes or the functional status of the blood sample obtained from the subject with a plurality of functional statuses of the blood samples obtained from control subjects is performed using clustering of the determined pathway activities, preferably by hierarchical clustering.

It was found by the inventors that a prediction regarding the mortality risk can be made based on the three or more target genes or the functional status of a blood sample obtained from a subject. By comparing the expression levels of the three or more target genes or the functional status of a blood sample from a subject with reference samples of septic subject which have died and which have survived, a likelihood can be calculated that the subject will die from sepsis. This prediction can be done by using the numeric values representing the different determined signaling pathway activities, e.g. the AR singling activity, or the combination of AR and TGFbeta signaling activity) and comparing these values with the values obtained for the reference groups. Alternatively, this prediction can be directly based on the expression levels of the three or more genes. This comparison can be done using statistical methods or for example by using clustering. When using clustering the subject is predicted to have a high mortality risk when the subject is clustered together with the reference subjects which have died or is predicted to have low mortality risk when clustered together with reference subjects which have survived. Preferably, the prediction is made in the method according to the invention, wherein the method is performed on the blood sample obtained from a patient having sepsis. Preferably the prediction is made for a subject for which it has already been established that the subject has sepsis, for example by the methods described herein.

Preferably, the prediction is made in the method according to the invention, wherein said comparing of the expression levels of the three or more genes or the functional status of the blood sample obtained from the subject with a plurality of expression levels of three or more genes or functional statuses of the blood samples obtained from control subjects is performed using clustering of the determined pathway activities, preferably by hierarchical clustering. It was found by the inventors that by clustering based on the determined signaling activities samples obtained from septic patients can be identified that have dies as a result form sepsis. Therefore by comparing the determined signaling activities determined in a blood sample obtained from a subject with known reference patients the mortality risk can be predicted as high when the respective signaling activities cluster with patients that have died or the risk can be predicted as low when the respective signaling activities cluster with patients that survived.

Preferably for the purpose of predicting survival probability of a subject with sepsis, determining the expression level of three or more target genes of the AR signaling pathway, the TGFbeta signaling pathway, the MAPK-AP1 signaling pathway and/or the JAK-STAT3 signaling pathway comprises:
determining the expression level of three or more target genes, for example 3, 4, 5, 6, 7, 8, 9, 10, 11, 12 or more target genes of the AR signaling pathway wherein three or more target genes, e.g. 3, 4 or 5, are selected form the group consisting of: ELL2, FKBP5, EAF2, NDRG1 and DHCR24 and/or;
determining the expression level of three or more target genes, for example 3, 4, 5, 6, 7, 8, 9, 10, 11, 12 or more target genes of the TGFbeta signaling pathway wherein three or more target genes, e.g. 3, 4 or 5, are selected form the group consisting of: ID1, SKIL, GADD45A, HMGA2 and SMAD4, and/or;
determining the expression level of three or more target genes of the MAPK-AP1 signaling pathway comprises determining the expression level of three or more target genes, for example 3, 4, 5, 6, 7, 8, 9, 10, 11, 12 or more target genes of the MAPK-AP1 pathway wherein three or more target genes, e.g. 3, 4 or 5, are selected form the group consisting of: BCL2L11, EZR, ENPP2, MMP3 and PLAUR, and/or;
determining the expression level of three or more target genes, for example 3, 4, 5, 6, 7, 8, 9, 10, 11, 12 or more target genes of the JAK-STAT3 signaling pathway wherein three or more target genes, e.g. 3, 4 or 5, are selected form the group consisting of: BIRC5, HSP90B1, MMP3, IL10, HIF1A.

Therefore, in an embodiment the invention relates to a method wherein said expression levels of the three or more genes are used in predicting the mortality risk for the subject from which the blood sample has been obtained,
wherein said prediction is based on a comparison of the expression levels of the three or more genes of the subject with a plurality of reference expression levels of three or more genes obtained from reference subjects, wherein said plurality of reference expression levels of the three or more genes obtained from reference subjects comprises expression levels of the three or more genes obtained from subject with sepsis which is a non-survivor and expression levels of the three or more genes obtained from subject with sepsis which is a survivor, and optionally further comprises expression levels of the three or more genes obtained from a healthy or non-septic control subject,
wherein the subject from which the blood sample is obtained is confirmed to have sepsis, and
wherein a low mortality risk is predicted when the expression levels of the three or more genes obtained from the subject with sepsis are similar to expression levels of the three or more genes obtained from reference subject with sepsis which is a survivor or when the expression levels of the three or more genes obtained from the subject with sepsis are similar to the expression levels of the three or more genes obtained from the at least one healthy or non-septic control subject, and
wherein a high mortality risk is predicted when the expression levels of the three or more genes obtained from the subject with sepsis are similar to the expression levels of the three or more genes obtained from the reference subject with sepsis which is a non-survivor.

In an alternative embodiment the invention relates to a method for determining the mortality risk for a subject, based on RNA extracted from a blood sample form the subject, the method comprising the steps of:
determining or receiving the result of a determination of the expression level of three or more target genes of the AR pathway;
determining the AR signaling pathway activity, based on the determined expression levels of said three or more target genes of the AR signaling pathway;
and optionally:
determining or receiving the result of a determination of the expression level of three or more target genes of the TGFbeta pathway;
determining the TGFbeta signaling pathway activity, based on the determined expression levels of said three or more target genes of the TGFbeta signaling pathway;
determining or receiving the result of a determination of the expression level of three or more target genes of the MAPK-AP1 pathway;
determining the MAPK-AP1 signaling pathway activity, based on the determined expression levels of said three or more target genes of the MAPK-AP1 signaling pathway;
determining or receiving the result of a determination of the expression level of three or more target genes of the JAK-STAT3 pathway;
determining the JAK-STAT3 signaling pathway activity, based on the determined expression levels of said three or more target genes of the JAK-STAT3 signaling pathway;
wherein the signaling pathway activities in the blood sample is used in predicting the mortality risk for the subject from which the blood sample has been obtained,
wherein said prediction is based on a comparison of the signaling pathway activities in the blood sample of the subject with the signaling pathway activities in a plurality of blood samples obtained from reference subjects, wherein said signaling pathway activities in a plurality of blood samples obtained from reference subjects comprise at least one blood sample obtained from subject with sepsis which is a non-survivor and at least one blood samples obtained from subject with sepsis which is a survivor, and optionally further comprise at least one blood samples obtained from a healthy or non-septic control subject,
wherein the subject from which the blood sample is obtained is confirmed to have sepsis, and
wherein a low mortality risk is predicted when the signaling pathway activity in the blood sample obtained from the subject with sepsis is similar to the signaling pathway activity in at least one blood sample obtained from reference subject with sepsis which is a survivor or when the signaling pathway activity in the blood sample obtained from the subject with sepsis is similar to the signaling pathway activity in at least one of the blood samples obtained from the healthy or non-septic control subject, and
wherein a high mortality risk is predicted when the signaling pathway activity in the blood sample obtained from the subject with sepsis is similar to the signaling pathway activity in at least one blood sample obtained from the reference subject with sepsis which is a non-survivor.

In an embodiment said comparing of the expression levels of the three or more genes or the signaling pathway activities in the blood sample obtained from the subject with a the expression levels of the three or more genes or the signaling pathway activities in a plurality of blood samples obtained from control subjects is performed using clustering of the determined pathway activities, preferably by hierarchical clustering.

In a further embodiment the subject from which the blood sample has been obtained does not have sepsis, and wherein the expression levels of the three or more genes are used to determine the risk that the subject will develop sepsis,
the method further comprising comparing the expression levels of the three or more genes of the subject from which the blood sample has been obtained to expression levels of the three or more genes obtained from a healthy or non-septic control subject.

In an alternative embodiment, the subject from which the blood sample has been obtained does not have sepsis, and wherein the functional status of the blood sample is used to determine the risk that the subject will develop sepsis,
the method further comprising comparing the functional status of the blood sample of the subject from which the blood sample has been obtained to at least one functional status of a blood sample obtained from a healthy or non-septic control subject,
preferably wherein the subject from which the blood sample has been obtained is predicted to be at risk to develop sepsis if the functional status of the blood sample comprises an AR signaling pathway activity which AR signaling pathway activity is determined to be higher than the AR signaling pathway activity determined in the control blood sample obtained from a healthy or non-septic control subject. In a preferred embodiment the predicting the risk to develop sepsis for a subject that does not have sepsis is further based on the TGFbeta signaling pathway activity wherein the subject is at risk to develop sepsis when the TGFbeta signaling pathway activity is determined to be higher than the TGFbeta signaling pathway activity determined in the control blood sample obtained from a healthy or non-septic control subject. In a more preferred embodiment the subject from which the blood sample has been obtained does not have sepsis, is determined to be at risk to develop sepsis when both the AR and the TGFbeta signaling pathway activities are determined to be higher than the AR and TGFbeta signaling pathway activities determined in the control blood sample obtained from a healthy or non-septic control subject. Preferably the subject that does not have sepsis is a subject with a bacterial infection.

It was found by the inventors that in patients that do not have sepsis but are at risk to develop sepsis, e.g. patients with a bacterial infection, patients with a high risk to develop sepsis can be identified as high risk correlates with increased AR and optionally TGFbeta signaling pathway activity.

Therefore, the invention further relates to a method for determining the risk for a non-septic subject to develop sepsis, based on RNA extracted from a blood sample form the subject, the method comprising the steps of:
determining or receiving the result of a determination of the expression level of three or more target genes of the AR pathway;
determining the AR signaling pathway activity, based on the determined expression levels of said three or more target genes of the AR signaling pathway;
and optionally:
determining or receiving the result of a determination of the expression level of three or more target genes of the TGFbeta pathway;
determining the TGFbeta signaling pathway activity, based on the determined expression levels of said three or more target genes of the TGFbeta signaling pathway; and/or
determining or receiving the result of a determination of the expression level of three or more target genes of the MAPK-AP1 pathway;
determining the MAPK-AP1 signaling pathway activity, based on the determined expression levels of said three or more target genes of the MAPK-AP1 signaling pathway; and/or
determining or receiving the result of a determination of the expression level of three or more target genes of the JAK-STAT3 pathway;
determining the JAK-STAT3 signaling pathway activity, based on the determined expression levels of said three or more target genes of the JAK-STAT3 signaling pathway;
wherein the signaling pathway activities in the blood sample is used to determine the risk that the subject will develop sepsis,
wherein the subject from which the blood sample has been obtained does not have sepsis,
the method further comprising comparing the signaling pathway activity in the blood sample of the subject from which the blood sample has been obtained to the signaling pathway activity in at least one blood sample obtained from a healthy or non-septic control subject.

Preferably the subject that does not have sepsis is a subject with a bacterial infection.

In an embodiment the subject from which the blood sample has been obtained is predicted to be at risk to develop sepsis if the AR signaling pathway activity is determined to be higher than the AR signaling pathway activity determined in the control blood sample obtained from a healthy or non-septic control subject.

In a further embodiment of the invention the subject from which the blood sample has been obtained has recovered from sepsis, and wherein the expression levels of the three or more genes of the blood sample are used to monitor the risk that the subject will develop a recurrence of sepsis, the method further comprising comparing the expression levels of the three or more genes of the subject from which the blood sample has been obtained to expression levels of the three or more genes obtained from a healthy or non-septic control subject.

In an alternative embodiment, the subject from which the blood sample has been obtained has recovered from sepsis, and wherein the functional status of the blood sample is used to monitor the risk that the subject will develop a recurrence of sepsis,
the method further comprising comparing the functional status of the blood sample of the subject from which the blood sample has been obtained to at least one functional status of a blood sample obtained from a healthy or non-septic control subject,
preferably wherein the subject from which the blood sample has been obtained is predicted to be at risk to develop a recurrence of sepsis if the functional status of the blood sample comprises an AR signaling pathway activity which AR signaling pathway activity is determined to be higher than the AR signaling pathway activity determined in the control blood sample obtained from a healthy or non-septic control subject. In a preferred embodiment the predicting the risk to develop a recurrence of sepsis for a subject that has recovered from sepsis is further based on the TGFbeta signaling pathway activity wherein the subject is at risk to develop a recurrence of sepsis when the TGFbeta signaling pathway activity is determined to be higher than the TGFbeta signaling pathway activity determined in the control blood sample obtained from a healthy or non-septic control subject. In a more preferred embodiment the subject that has recovered from sepsis is determined to be at risk to develop a recurrence of sepsis when both the AR and the TGFbeta signaling pathway activities are determined to be higher than the AR and TGFbeta signaling pathway activities determined in the control blood sample obtained from a healthy or non-septic control subject.

It was found that after recovery of sepsis patients remain susceptible to develop a recurrence of sepsis for a prolonged time. The inventors demonstrate that higher risk of recurrence correlates with increased AR and TGFbeta signaling pathway activities, and that based on AR signaling pathway activity alone or when combined with TGFbeta signaling pathway activity subject with a risk of developing a recurrence of sepsis can be identified.

Therefore, in a preferred embodiment the invention relates to a method for determining the risk for recurrence for a subject recovered from sepsis, based on RNA extracted from a blood sample form the subject, the method comprising the steps of:
determining or receiving the result of a determination of the expression level of three or more target genes of the AR pathway;
determining the AR signaling pathway activity, based on the determined expression levels of said three or more target genes of the AR signaling pathway;
and optionally:
determining or receiving the result of a determination of the expression level of three or more target genes of the TGFbeta pathway;
determining the TGFbeta signaling pathway activity, based on the determined expression levels of said three or more target genes of the TGFbeta signaling pathway; and/or
determining or receiving the result of a determination of the expression level of three or more target genes of the MAPK-AP1 pathway;
determining the MAPK-AP1 signaling pathway activity, based on the determined expression levels of said three or more target genes of the MAPK-AP1 signaling pathway; and/or
determining or receiving the result of a determination of the expression level of three or more target genes of the JAK-STAT3 pathway;
determining the JAK-STAT3 signaling pathway activity, based on the determined expression levels of said three or more target genes of the JAK-STAT3 signaling pathway;
wherein the subject from which the blood sample has been obtained has recovered from sepsis, and wherein the signaling pathway activity in the blood sample is used to monitor the risk that the subject will develop a recurrence of sepsis,
the method further comprising comparing the signaling pathway activity in the blood sample of the subject from which the blood sample has been obtained to the signaling pathway activity in at least one blood sample obtained from a healthy or non-septic control subject.

In an embodiment the subject from which the blood sample has been obtained is predicted to be at risk to develop a recurrence of sepsis if the AR signaling pathway activity is determined to be higher than the AR signaling pathway activity determined in the control blood sample obtained from a healthy or non-septic control subject.

According to a preferred embodiment of the present invention the blood sample is a whole blood sample, isolated peripheral blood mononuclear cells (PBMCs), isolated CD4+ cells, isolated CD8+ cells, Regulatory T-cells, mixed CD8+ and T cells, myeloid derived suppressor cells (MDSC), dendritic cells, isolated neutrophils, isolated lymphocytes or isolated monocytes.

In an embodiment of the present invention said signaling pathway activity or signaling pathway activities is determined based on evaluating a calibrated mathematical model relating the to the three or more expression levels determined for the pathway or pathways based on the RNA extracted from a blood sample to the activity or activities of the signaling pathway or signaling pathways.

According to a preferred embodiment of the invention, the functional status of a blood sample is determined based on evaluating a calibrated mathematical model relating the activities of the signaling pathways in the blood sample to a numeric value. This model may be programmed to interpret the combination of pathway activities so as to determine the functional status of the blood sample of the subject to be diagnosed, and optionally further use this functional status to provide a diagnosis or mortality risk. In particular, the determination of the functional status of a blood sample comprises (i) receiving activity of the respective signaling pathways in the blood sample of the subject to be diagnosed, (ii) determining the functional status of the blood sample of said subject, the determining being based on evaluating a calibrated mathematical model relating the activity of the respective signaling pathways to the functional status of the blood sample.

The calibrated mathematical pathway model is preferably a centroid or a linear model, or a Bayesian network model based on conditional probabilities. For example, the calibrated mathematical pathway model may be a probabilistic model, preferably a Bayesian network model, based on conditional probabilities relating the functional status of the blood sample and the activities of the signaling pathways, or the calibrated mathematical pathway model may be based on one or more linear combination(s) of the activities of the signaling pathways.

In accordance with the mathematical model, the activities of the signaling pathways are interpreted to provide the functional status of the blood sample, which may further be translated into the diagnosis, or are interpreted to provide directly the diagnosis. The functional status of the blood sample predicts or provides a probability that a subject has sepsis, and/or the probability that a subject with sepsis will die as a consequence of septic shock.

Accordingly, the determining of the diagnosis or determining the mortality risk may comprise determining functional status of the blood sample based on the combination of the activities of the cellular signaling pathways in the blood sample and translating the functional status into the diagnosis or mortality risk. According to a preferred embodiment of the present invention, the activity of the respective signal pathway is determined or determinable by pathway analysis as described herein.

Accordingly, in a preferred embodiment of the invention, the method comprises a step of providing a blood sample obtained from a subject, and extracting RNA from said blood sample.

In an embodiment of the invention the subject is a pediatric subject.

In a second aspect, the present invention relates to a computer-implemented method for implementing the method of the first aspect of the invention and various embodiment thereof.

In accordance with a third aspect, the present invention relates to an apparatus for determining the functional status of a blood sample, and/or diagnosing a subject with sepsis, and/or predicting the mortality risk for the subject, the apparatus comprising a digital processor configured to perform the method of the first aspect of the present invention and the various embodiments thereof. In a preferred embodiment the invention relates to an apparatus for determining the functional status of a blood sample, the apparatus comprising a digital processor configured to perform the method according to any one of the preceding claims, comprising an input adapted to receive data indicative of a target gene expression profile for the three or more target genes of the AR signaling pathway, optionally data indicative of a target gene expression profile for the three or more target genes of the TGFbeta signaling pathway and/or the MAPK-AP1 signaling pathway and/or the JAK-STAT3 signaling pathway

In accordance with a fourth aspect, the present invention relates to a non-transitory storage medium for determining the functional status of a blood sample, and/or diagnosing a subject with sepsis, and/or predicting the mortality risk for the subject, the non-transitory storage medium storing instructions that are executable by a digital processing device to perform the method of the first aspect of the present invention and the various embodiments thereof. In a preferred embodiment the invention relates to a computer program product comprising instructions which, when the program is executed by a computer, cause the computer to carry out a method comprising:
receiving data indicative of a target gene expression profile for three or more target genes of the AR signaling pathway, optionally further receiving data indicative of the target gene expression levels of three or more target genes of the TGFbeta signaling pathway and/or the MAPK-AP1 signaling pathway and/or the JAK-STAT3 signaling pathway,
determining the AR signaling pathway activity, and optionally TGFbeta signaling pathway activity and/or the MAPK-AP1 signaling pathway activity and/or the JAK-STAT3 signaling pathway activity based on the determined expression levels of said three or more target genes of the AR signaling pathway and optionally the TGFbeta signaling pathway and/or the MAPK-AP1 signaling pathway and/or the JAK-STAT3 signaling pathway,
determining the functional status of the blood sample based on the determined AR signaling pathway activity and optionally TGFbeta signaling pathway activity and/or the MAPK-AP1 signaling pathway activity and/or the JAK-STAT3 signaling pathway activity, wherein said functional status of said blood sample is being determined as having the determined AR signaling pathway activity and optionally the TGFbeta signaling pathway activity and/or the MAPK-AP1 signaling pathway activity and/or the JAK-STAT3 signaling pathway activity, and
optionally providing a diagnosis or prediction based on the functional status of the blood sample.

The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

In accordance with a fifth aspect, the present invention relates to a computer program for determining the functional status of a blood sample, and/or diagnosing a subject with sepsis, and/or predicting the mortality risk for the subject, the computer program comprising program code means for causing a digital processing device to perform a method according to the first aspect of the present invention and the various embodiments thereof, when the computer program is run on the digital processing device. The computer program may be stored/distributed on a suitable medium, such as an optical storage medium or a solid-state medium, supplied together with or as part of other hardware, but may also be distributed in other forms, such as via the Internet or other wired or wireless telecommunication systems.

In a sixth aspect the invention relates to a of parts, comprising primers and optionally probes for determining the expression levels of three or more, for example three, four, five, six, seven, eight, nine, ten, eleven, twelve or more, genes,
wherein the three or more, for example three, four, five, six, seven, eight, nine, ten, eleven, twelve or more, genes are selected from group 1 and group 2, wherein group 1 consists of: ABCC4, APP, AR, CDKN1A, CREB3L4, DHCR24, EAF2, ELL2, FGF8, FKBP5, GUCY1A3, IGF1, KLK2, KLK3, LCP1, LRIG1, NDRG1, NKX3_1, NTS, PLAU, PMEPA1, PPAP2A, PRKACB, PTPN1, SGK1, TACC2, TMPRSS2, and UGT2B1_5, and
wherein group 2 consists of: ANGPTL4, CDC42EP3, CDKN1A, CDKN2B, CTGF, GADD45A, GADD45B, HMGA2, ID1, IL11, INPP5D, JUNB, MMP2, MMP9, NKX2_5, OVOL1, PDGFB, PTHLH, SERPINE1, SGK1, SKIL, SMAD4, SMAD5, SMAD6, SMAD7, SNAI1, SNAI2, TIMP1 and VEGFA.

Designing primers and probes is a routine technology in the field of gene detection and quantification. Primers may for example be designed by online programs such as Primer3 (https://primer3.ut.ee/). Probes for qPCR are typically designed to bind the amplification product and have a fluorescent part and a quencher, allowing to distinguish between bound and unbound state. The genomic sequences for the above genes can easily be found in genomic databases such as the UCSC Genome Browser, Ensembl Genome Browser or NCBI Genome Data Viewer.

In an embodiment group 1 consists of the genes AR, CREB3L4, DHCR24, EAF2, ELL2, FKBP5, GUCY1A3, IGF1, KLK3, LCP1, LRIG1, NDRG1, NKX3_1, PMEPA1, PRKACB, TMPRSS2, preferably AR, CREB3L4, DHCR24, EAF2, ELL2, FKBP5, LCP1, LRIG1, NDRG1, PMEPA1, PRKACB, TMPRSS2 more preferably DHCR24, EAF2, ELL2, FKBP5, LCP1, LRIG1, PMEPA1, PRKACB, and/or group 2 consists of the genes CDC42EP3, GADD45A, GADD45B, HMGA2, ID1, IL11, INPP5D, JUNB, MMP2, MMP9, NKX2_5, OVOL1, PDGFB, PTHLH, SGK1, SKIL, SMAD4, SMAD5, SMAD6, TIMP1, VEGFA, preferably CDC42EP3, GADD45A, GADD45B, ID1, JUNB, MMP9, PDGFB, SGK1, SKIL, SMAD5, SMAD6, TIMP1, VEGFA, more preferably CDC42EP3, GADD45A, GADD45B, ID1, JUNB, MMP9, PDGFB, SGK1, SMAD5, TIMP1, VEGFA. In an embodiment the three or more genes are selected from group 1.

In an alternative embodiment he invention further relates to a kit of parts, comprising primers for inferring activity of one or more cellular signaling pathway(s) by determining the expression levels of one or more set(s) of target genes of the respective cellular signaling pathway(s), wherein the cellular signaling pathway(s) comprise(s) a AR pathway, and optionally further comprises one or more of an TGFbeta pathway, an MAPK-AP1 pathway and a JAK-STAT3 pathway,
wherein the set of target genes of the AR pathway comprises three or more target genes, for example 3, 4, 5, 6, 7, 8, 9, 10, 11, 12 or more target genes selected from the group comprising: KLK2, PMEPA1, TMPRSS2, NKX3_1, ABCC4, KLK3, FKBP5, ELL2, UGT2B15, DHCR24, PPAP2A, NDRG1, LRIG1, CREB3L4, LCP1, GUCY1A3, AR and EAF2, preferably wherein the set of target genes of the AR pathway comprises three or more target genes, for example 3, 4, 5, 6, 7, 8, 9, 10, 11 or 12 target genes selected from the group consisting of ELL2, FKBP5, GUCY1A3, LRIG1, PLAU, PMEPA1, PRKACB, SGK1, NDRG1, CREB3L4, DHCR24 or PTPN1, and
wherein the set of target genes of the TGFbeta pathway comprises three or more target genes, for example 3, 4, 5, 6, 7, 8, 9, 10, 11, 12 or more target genes selected from the group comprising: ANGPTL4, CDC42EP3, CDKN1A, CTGF, GADD45A, GADD45B, HMGA2, ID1, IL11, JUNB, PDGFB, PTHLH, SERPINE1, SGK1, SKIL, SMAD4, SMAD5, SMAD6, SMAD7, SNAI2, VEGFA, preferably wherein the set of target genes of the TGFbeta pathway comprises three or more target genes, for example 3, 4, 5, 6, 7, 8, 9, 10, 11 or 12 target genes selected from the group consisting of CDC42EP3, GADD45A, ID1, MMP9, SGK1, SMAD5, SMAD7, VEGFA, JUNB, TIMP1, SKIL and CCKN1A, and
wherein the set of target genes of the MAPK-AP1 pathway comprises three or more target genes, for example 3, 4, 5, 6, 7, 8, 9, 10, 11, 12 or more target genes selected from the group comprising: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM, preferably wherein the set of target genes of the MAPK-AP1 pathway comprises three or more target genes, for example 3, 4, 5, 6, 7, 8, 9, 10, or 11 target genes selected from the group consisting of DNMT1, EGFR, ENPP2, GLRX, MMP9, PLAUR, TIMP1, LOR, EZR, DDIT3 and TP53, and
wherein the set of target genes of the JAK-STAT3 pathway comprises three or more target genes, for example 3, 4, 5, 6, 7, 8, 9, 10, 11, 12 or more target genes selected from the group comprising: AKT1, BCL2, BCL2L1, BIRC5, CCND1, CD274, CDKN1A, CRP, FGF2, FOS, FSCN1, FSCN2, FSCN3, HIF1A, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSF1B, TWIST1, VIM, and ZEB1 preferably wherein the set of target genes of the JAK-STAT3 pathway comprises three or more target genes, for example 3, 4, 5, 6, 7, 8, 9, 10, 11, 12 or more target genes selected from the group consisting of BCL2, BIRC5, CD274, FOS, HSPA1A, JUNB, MMP9, STAT1, TIMP1, BCL2L1, HSPA1B, HSP90AB1, HSP90B1, POU2F1 and ICAM1.

In a preferred embodiment of the invention the kit of parts further comprises the apparatus according to the third aspect of the invention, and/or the non-transitory storage product according to the fourth aspect of the invention, and/or the computer program according to the fifth aspect of the invention.

In an embodiment the invention relates to use of the kit according to the sixth aspect in a method according the first aspect.

In a seventh aspect the invention relates to a method for in vitro or ex vivo diagnosing or prognosticating whether a subject has sepsis, has septic shock or has a high mortality risk as a result of sepsis using the kit according to the sixth aspect of the invention. Preferably wherein: an increased expression of ABCC4, APP, FGF8, FKBP5, ELL2, DHCR24, NDRG1, LCP1, EAF2, PTPN1, CDC42EP3, CDKN2B, CTGF, GADD45A, GADD45B, HMGA2, ID1, IGF1, IL11, INPP5D, JUNB, MMP9, PTHLH, SERPINE1, SGK1, SKIL, SMAD4, SMAD6, SNAI2, TIMP1 and VEGFA; or a decreased expression of CDKN1A, KLK2, KLK3, PMEPA1, TMPRSS2, NKX2_5, NKX3_1, NTS, PLAU, UGT2B15, PPAP2A, LRIG1, TACC2, CREB3L4, GUCY1A3, AR, ANGPTL4, MMP2, OVOL1, PDGFB, PRKACB, SMAD5, SMAD7 and SNAI1 correlates with sepsis.

In an alternative embodiment, the invention relates to a method for in vitro or ex vivo diagnosing or prognosticating whether a subject has sepsis, has septic shock or has a high mortality risk as a result of sepsis using a kit, the kit comprising primers for inferring activity of one or more cellular signaling pathway(s) by determining the expression levels of one or more set(s) of target genes of the respective cellular signaling pathway(s), wherein the cellular signaling pathway(s) comprise(s) a AR pathway, and optionally further comprises one or more of an TGFbeta pathway, an MAPK-AP1 pathway and a JAK-STAT3 pathway,
wherein the set of target genes of the AR pathway comprises three or more target genes selected from the group comprising: KLK2, PMEPA1, TMPRSS2, NKX3_1, ABCC4, KLK3, FKBP5, ELL2, UGT2B15, DHCR24, PPAP2A, NDRG1, LRIG1, CREB3L4, LCP1, GUCY1A3, AR and EAF2, and
wherein the set of target genes of the TGFbeta pathway comprises three or more target genes selected from the group comprising: ANGPTL4, CDC42EP3, CDKN1A, CTGF, GADD45A, GADD45B, HMGA2, ID1, IL11, JUNB, PDGFB, PTHLH, SERPINE1, SGK1, SKIL, SMAD4, SMAD5, SMAD6, SMAD7, SNAI2, VEGFA, and
wherein the set of target genes of the MAPK-AP1 pathway comprises three or more target genes selected from the group comprising: BCL2L11, CCND1, DDIT3, DNMT1, EGFR, ENPP2, EZR, FASLG, FIGF, GLRX, IL2, IVL, LOR, MMP1, MMP3, MMP9, SERPINE1, PLAU, PLAUR, PTGS2, SNCG, TIMP1, TP53, and VIM, and
wherein the set of target genes of the JAK-STAT3 pathway comprises three or more target genes selected from the group comprising: AKT1, BCL2, BCL2L1, BIRC5, CCND1, CD274, CDKN1A, CRP, FGF2, FOS, FSCN1, FSCN2, FSCN3, HIF1A, HSP90AA1, HSP90AB1, HSP90B1, HSPA1A, HSPA1B, ICAM1, IFNG, IL10, JunB, MCL1, MMP1, MMP3, MMP9, MUC1, MYC, NOS2, POU2F1, PTGS2, SAA1, STAT1, TIMP1, TNFRSFlB, TWIST1, VIM, and ZEB1.

This application describes several preferred embodiments. Modifications and alterations may occur to others upon reading and understanding the preceding detailed description. It is intended that the application is construed as including all such modifications and alterations insofar as they come within the scope of the appended claims or the equivalents thereof.

Other variations to the disclosed embodiments can be understood and effected by those skilled in the art in practicing the claimed invention, from a study of the drawings, the disclosure, and the appended claims.

It shall be understood that the methods of the first aspect, the computer implemented invention of the second aspect, the apparatus of the third aspect, the non-transitory storage medium of fourth aspect, the computer program of the fifth aspect, the kits of the sixth aspect have similar and/or identical preferred embodiments, in particular, as defined in the dependent claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Calculations like the determination of the mortality risk performed by one or several units or devices can be performed by any other number of units or devices.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

General: In all the figures where signal transduction pathway analysis scores are depicted, these are given as log 2odds scores for pathway activity, derived from the probability scores for pathway activity provided by the Bayesian pathway model analysis. Log2odds scores indicate the level of activity of a signaling pathway on a linear scale.

Analyzed public datasets are indicated with their GSE number (in principle at the bottom of each figure), and individual samples with their GSM number (in principle most right column for clustering diagrams).

All validation samples for a signaling pathway model or an immune response/system model are independent samples and have not been used for calibration of the respective model to be validated.

FIG. 1 shows AR signaling pathway activity (top) and TGFbeta signaling pathway activity (bottom) for septic shock patients and healthy control subjects from dataset GSE26440. The data are obtained from whole blood samples from septic shock patients (survivors), septic shock patients (non-survivors), control subject (healthy subjects) and control subjects (non-septic survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 2 shows MPK-AP1 signaling pathway activity (top) and JAK-STAT3 signaling pathway activity (bottom) for septic shock patients and healthy control subjects from dataset GSE26440. The data are obtained from whole blood samples from septic shock patients (survivors), septic shock patients (non-survivors), control subject (healthy subjects) and control subjects (non-septic survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, * depicts a p value of 1.00e\hat{ }-04<p<=1.00e-03, and **** depicts a p value of p<=1.00e-04.

FIG. 3 shows AR signaling pathway activity (top) and TGFbeta signaling pathway activity (bottom) for septic shock patients and healthy control subjects from dataset GSE4607. The data are obtained from whole blood samples from control subjects, septic shock patients (Non-survivors) and septic shock patients (Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 4 shows MPK-AP1 signaling pathway activity (top) and JAK-STAT3 signaling pathway activity (bottom) for septic shock patients and healthy control subjects from dataset GSE4607. The data are obtained from whole blood samples from control subjects, septic shock patients (Non-survivors) and septic shock patients (Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 5 shows AR signaling pathway activity (top) and TGFbeta signaling pathway activity (bottom) for septic shock patients and healthy control subjects from dataset GSE66099. The data are obtained from whole blood samples from control subjects, septic shock patients (Non-survivors) and septic shock patients (Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 6 shows MPK-AP1 signaling pathway activity (top) and JAK-STAT3 signaling pathway activity (bottom) for septic shock patients and healthy control subjects from dataset GSE66099. The data are obtained from whole blood samples from control subjects, septic shock patients (Non-survivors) and septic shock patients (Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 7 shows AR signaling pathway activity (top) and TGFbeta signaling pathway activity (bottom) for septic shock patients and healthy control subjects from dataset GSE95233. The data are obtained from whole blood samples from control subjects (CS=healthy control subject; PC=non-septic patient control), septic shock patients (NS=Non-survivors) and septic shock patients (SV=Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, * depicts a p value of 1.00e\hat{ }-04<p<=1.00e-03, and **** depicts a p value of p<=1.00e-04.

FIG. 8 shows MPK-AP1 signaling pathway activity (top) and JAK-STAT3 signaling pathway activity (bottom) for septic shock patients and healthy control subjects from dataset GSE95233. The data are obtained from whole blood samples from control subjects (CS=healthy control subject; PC=non-septic patient control), septic shock patients (NS=Non-survivors) and septic shock patients (SV=Survivors). The graphs show the log 2odds for the respective signaling pathway activity; statistical differences are indicated above the bars, where “ns” (not significant) depicts a p-value of 5.00e\hat{ }-02<p<=1.00e+00, * depicts a p value of 1.00e\hat{ }-02<p<=5.00e-02, ** depicts a p value of 1.00e\hat{ }-03<p<=1.00e-02, *** depicts a p value of 1.00e\hat{ }-04<p<=1.00e-03, and **** depicts a p value of p<=1.00e-04.

FIG. 9 shows a clustering diagram for the individual samples in dataset GSE26440 based on the AR and TGFbeta signaling pathways. The greyscale coding represents a logarithmic scale for the individual pathway scores. Hierarchical clustering was used. The color coding on the left side depicts: black=septic shock patient (survivor); light grey=septic shock patient (non-survivor); medium grey=normal control; dark grey=control survivor.

FIG. 10 shows a clustering diagram for the individual samples in dataset GSE26440 based on the AR, TGFbeta and MAPK-AP1 signaling pathways. The greyscale coding represents a logarithmic scale for the individual pathway scores. Hierarchical clustering was used. The color coding on the left side depicts: black=septic shock patient (survivor); light grey=septic shock patient (non-survivor); medium grey=normal control; dark grey=control survivor.

FIG. 11 shows a clustering diagram for the individual samples in dataset GSE26440 based on the AR, TGFbeta, MAPK-AP1 and JAK-STAT3 signaling pathways. The greyscale coding represents a logarithmic scale for the individual pathway scores. Hierarchical clustering was used. The color coding on the left side depicts: black=septic shock patient (survivor); light grey=septic shock patient (non-survivor); medium grey=normal control; dark grey=control survivor.

FIG. 12 shows a clustering diagram for the individual samples in dataset GSE4607 based on the AR and TGFbeta signaling pathways. The greyscale coding represents a logarithmic scale for the individual pathway scores. Hierarchical clustering was used. The color coding on the left side depicts: black=control; light grey=septic shock patient (non-survivor); dark grey=septic shock patient (survivor).

FIG. 13 shows a clustering diagram for the individual samples in dataset GSE4607 based on the AR, TGFbeta and MAPK-AP1 signaling pathways. The greyscale coding represents a logarithmic scale for the individual pathway scores. Hierarchical clustering was used. The color coding on the left side depicts: black=control; light grey=septic shock patient (non-survivor); dark grey=septic shock patient (survivor).

FIG. 14 shows a clustering diagram for the individual samples in dataset GSE4607 based on the AR, TGFbeta, MAPK-AP1 and JAK-STAT3 signaling pathways. The greyscale coding represents a logarithmic scale for the individual pathway scores. Hierarchical clustering was used. The color coding on the left side depicts: black=control; light grey=septic shock patient (non-survivor); dark grey=septic shock patient (survivor).

FIG. 15 shows a clustering diagram for the individual samples in dataset GSE66099 based on the AR and TGFbeta signaling pathways. The greyscale coding represents a logarithmic scale for the individual pathway scores. Hierarchical clustering was used. The color coding on the left side depicts: black=septic shock patient; light grey=septic patient; dark grey=control subject.

FIG. 16 shows a clustering diagram for the individual samples in dataset GSE66099 based on the AR, TGFbeta and MAPK-AP1 signaling pathways. The greyscale coding represents a logarithmic scale for the individual pathway scores. Hierarchical clustering was used. The color coding on the left side depicts: black=septic shock patient; light grey=septic patient; dark grey=control subject.

FIG. 17 shows a clustering diagram for the individual samples in dataset GSE66099 based on the AR, TGFbeta, MAPK-AP1 and JAK-STAT3 signaling pathways. The greyscale coding represents a logarithmic scale for the individual pathway scores. Hierarchical clustering was used. The color coding on the left side depicts: black=septic shock patient; light grey=septic patient; dark grey=control subject.

FIG. 18 shows a clustering diagram for the individual samples in dataset GSE95233 based on the AR and TGFbeta signaling pathways. The greyscale coding represents a logarithmic scale for the individual pathway scores. Hierarchical clustering was used. The color coding on the left side depicts: black=septic shock patient; light grey=septic patient; dark grey=control subject.

FIG. 19 shows a clustering diagram for the individual samples in dataset GSE95233 based on the AR, TGFbeta and MAPK-AP1 signaling pathways. The greyscale coding represents a logarithmic scale for the individual pathway scores. Hierarchical clustering was used. The color coding on the left side depicts: black=septic shock patient; light grey=septic patient; dark grey=control subject.

FIG. 20 shows a clustering diagram for the individual samples in dataset GSE95233 based on the AR, TGFbeta, MAPK-AP1 and JAK-STAT3 signaling pathways. The greyscale coding represents a logarithmic scale for the individual pathway scores. Hierarchical clustering was used. The color coding on the left side depicts: black=blood control; light grey=control survivor; medium grey=non-survivor day 1; dark grey=survivor day 1.

FIG. 21 depicts the pathway activities obtained from isolated THP-1 cells. THP-1 cells were incubated with H. pylori bacteria supernatant, directly incubated with H. pylori bacteria and compared with control THP-1 cells. Activities of the AR, ER, FOXO Hedgehog and TGFbeta pathways were determined and relative values are plotted.

FIG. 22 depicts the pathway activities obtained from isolated THP-1 cells. THP-1 cells were incubated with different concentrations of the bacterial product lipopolysaccharide (LPS) and compared with control THP-1 cells. Activities of the AR, ER, FOXO Hedgehog and TGFbeta pathways were determined and relative values are plotted.

FIG. 23 to FIG. 34. Boxplots shown predictive capacity of subsets of genes. Each FIG. depicts the random selection of N genes for N=1, 2, 3, 4, 5, or 6 for different subsets of the AR cellular signaling pathway (FIGS. 23 to 26), the TGFbeta cellular signaling pathway (FIGS. 27 to 30) or the combined target genes of the AR and the TGFbeta cellular signaling pathways (FIGS. 31 to 34). Either the entire set of target genes was used (T=0, FIGS. 23, 27 and 31) or a cutoff was used to select a subset of the target genes based on their contribution to the pathway activity score (T=0.3, 0.4 or 0.5, FIGS. 24-26, 28-30, 32-34). From each selected set of genes a random selection of N genes was made 1000 times, and the respective gene selections were used to determine whether sepsis patients can be distinguished from healthy subjects (at least 2 SD difference). The results are plotted in the form of box plots, where set 1 represents combined datasets GSE26440, GSE4607 and GSE66099, set 2 represents dataset GSE95233 and set 3 represents dataset GSE57065. The median is indicated by the thick line in the box, the 25^{th} percentile by the lower boundary of the box and the 10^{th} percentile by the dotted line.

FIG. 35 Schematic overview of AR inhibitor experiment. Depicts the experimental setup to determine if AR inhibitors can be used to mitigate the effect of LPS on monocytes (THP-1 cells). In brief, monocyte cells (THP-1) are cultured for 24 hours with or without LPS, after which the medium is changed and both conditions are subsequently cultured with or without DHT. Both LPS and DHT are anticipated to activate the AR cellular signaling pathway. In a parallel experiment, THP-1 cells are first cultured for 24 hours with LPS, after which the medium is changed and the cells are cultured with one of ARCC-4, ARD-266, A-458 and bicalutamide.

FIGS. 36 to 39 pathway activities as determined in the AR inhibitor experiment. FIGS. 36 to 39 describe the experimental outcome of the different conditions outlined in FIG. 35 in terms of measured cellular signaling pathway activities. The FIGS. depict the AR, ER, HH (Hedgehog) and TGFbeta cellular signaling pathway activities as determined in the different experimental groups respectively. Experiments were performed in triplicate, standard deviations of the measured activities are indicated in the graphs.

### DETAILED DESCRIPTION OF EMBODIMENTS

The following examples merely illustrate particularly preferred methods and selected aspects in connection therewith. The teaching provided herein may be used for constructing several tests and/or kits, e.g., to detect, predict and/or diagnose the functional status of one or more blood samples. Furthermore, upon using methods as described herein drug prescription can advantageously be guided, drug response prediction and monitoring of drug efficacy (and/or adverse effects) can be made. The following examples are not to be construed as limiting the scope of the present invention.

### Example 1—Methods and Sample Description

Using the Gene Expression Omnibus (GEO) database (https://www.ncbi.nlm.nih.gov/gds/) Affymetrix HG-U133Plus2.0 data from samples from clinical and preclinical studies in which whole blood samples (GSE26440, GSE4607, GSE66099, GSE95233, for more information about sample type and preparation see Table 1) were used. We used the pathway analysis to determine the signal transduction pathway activities (AR, ER, PR, GR, HH, Notch, TGFbeta, WNT, JAK-STAT1/2, JAK-STAT3, NFkB, PI3K, MAPK). For the hierarchical clustering we used the clustering tool Seaborn clustermap.

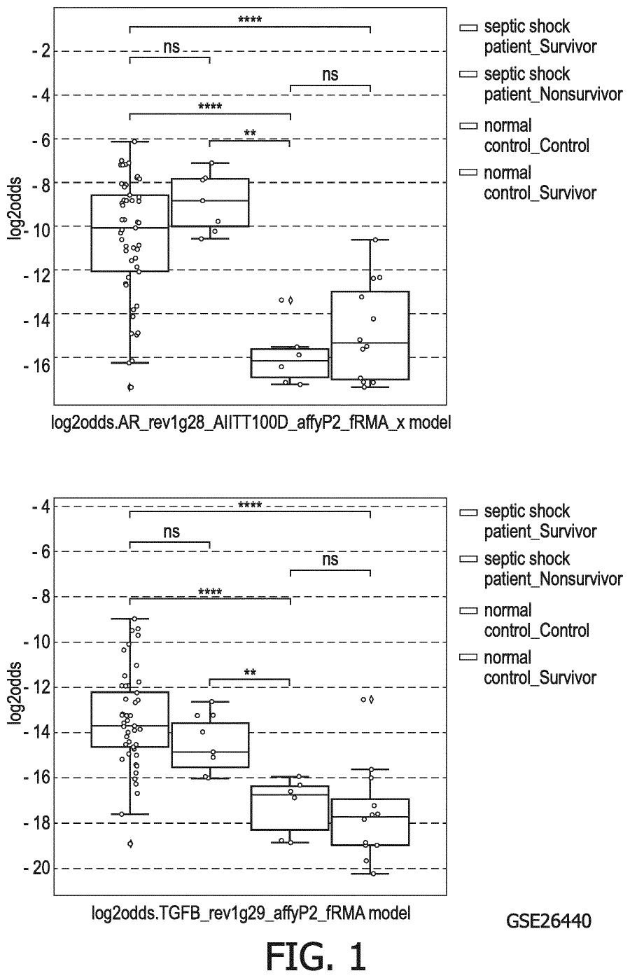

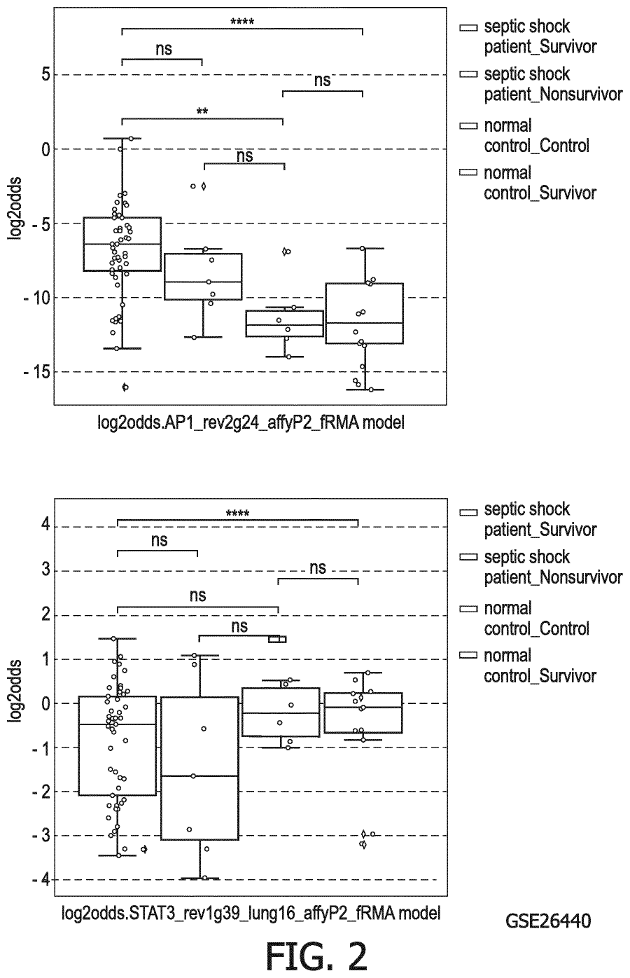

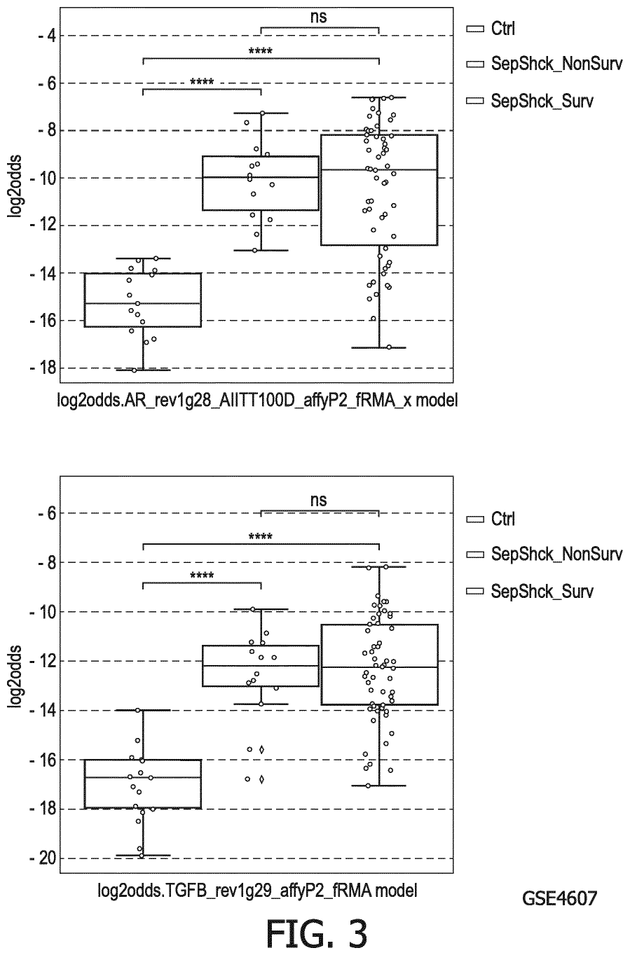

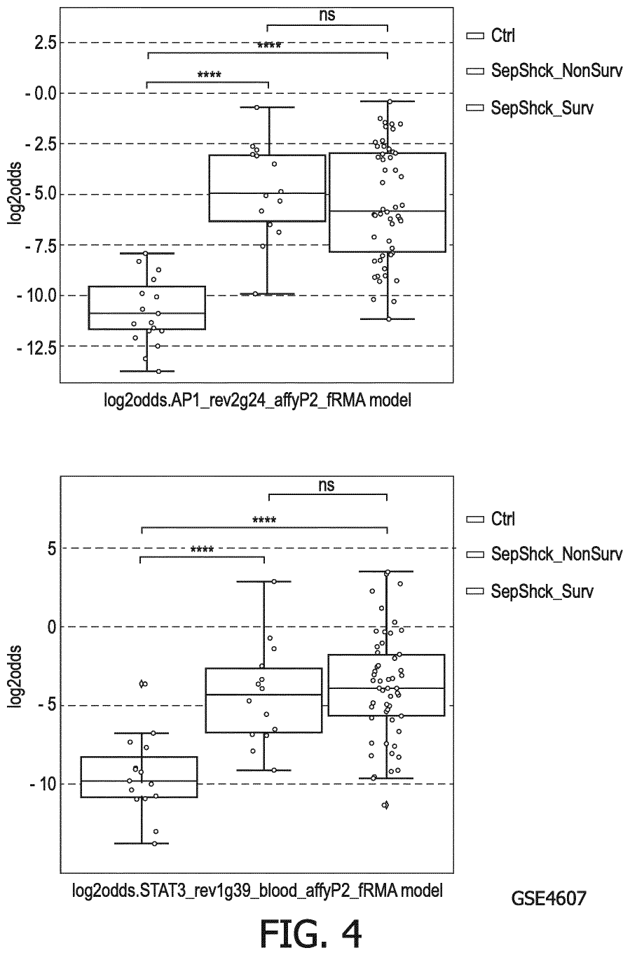

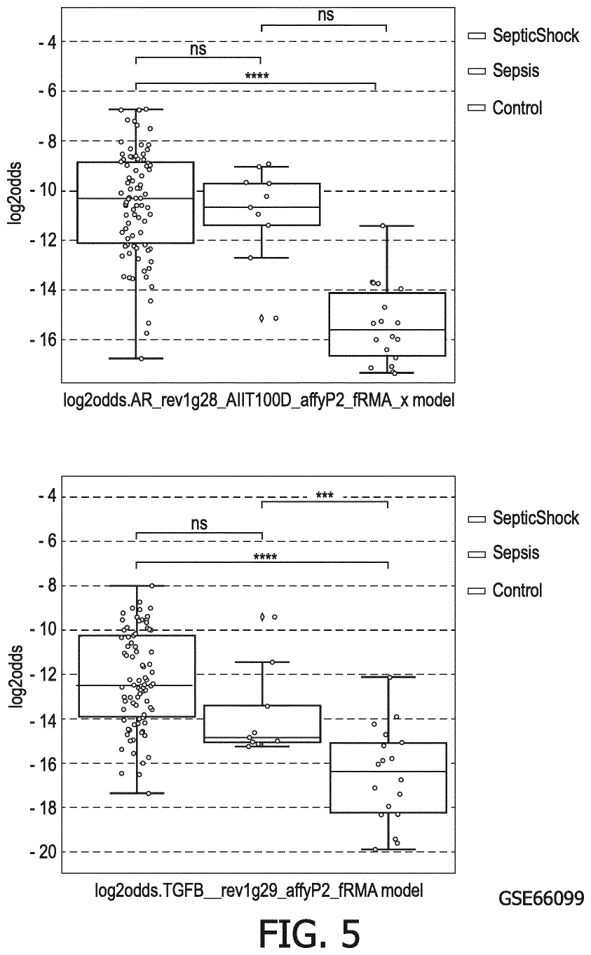

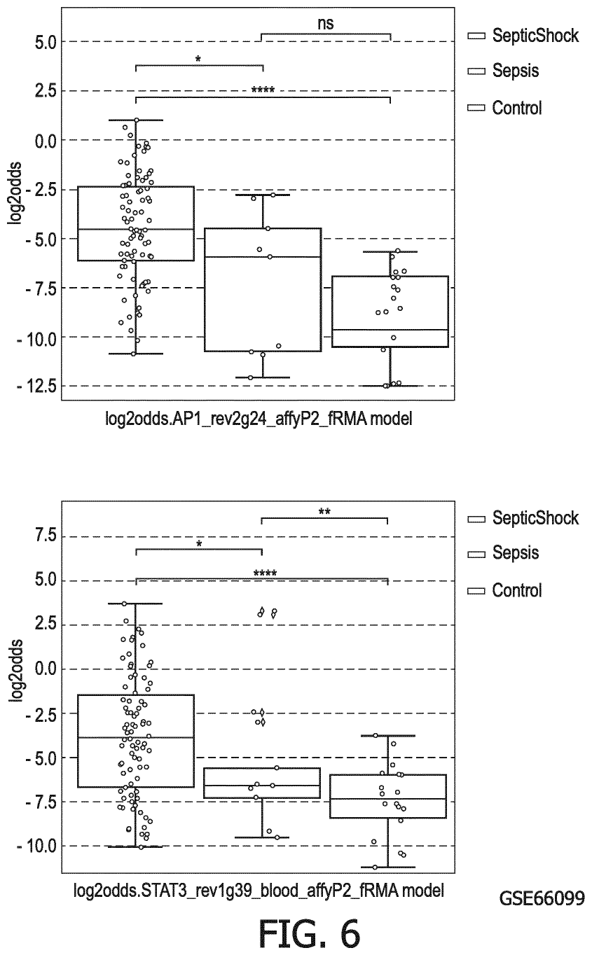

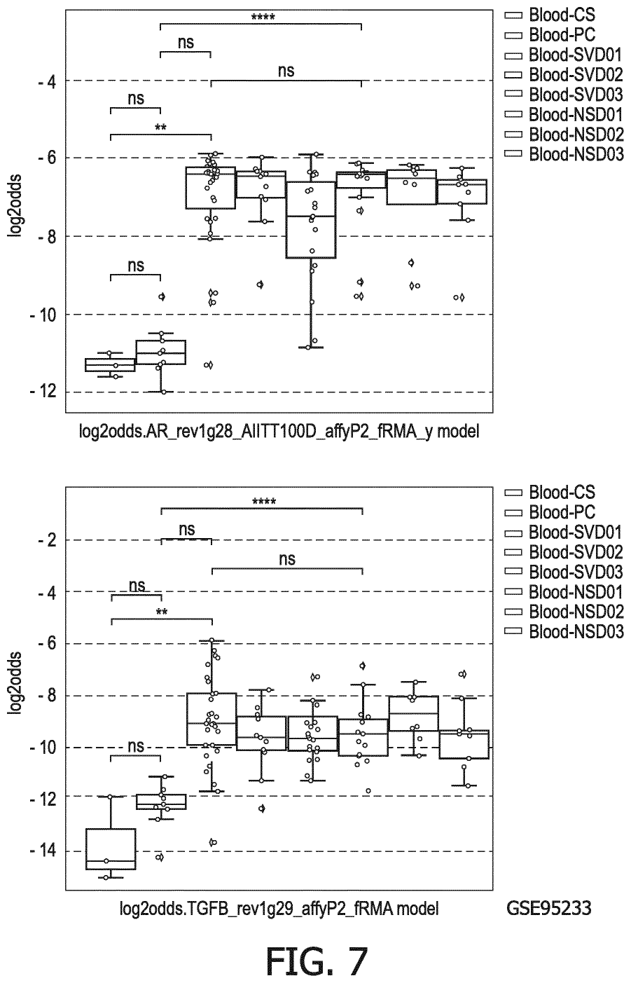

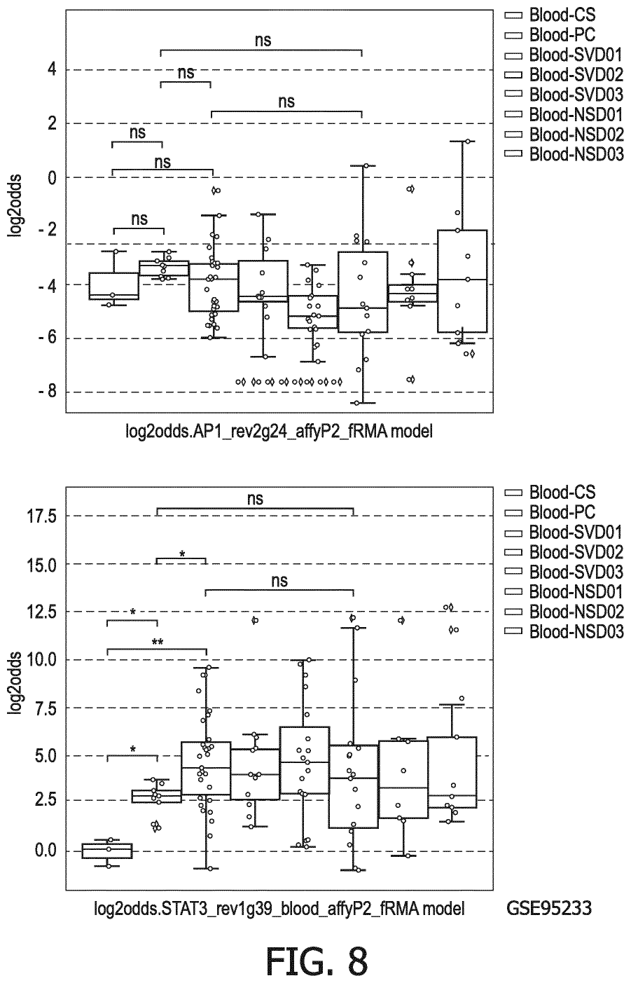

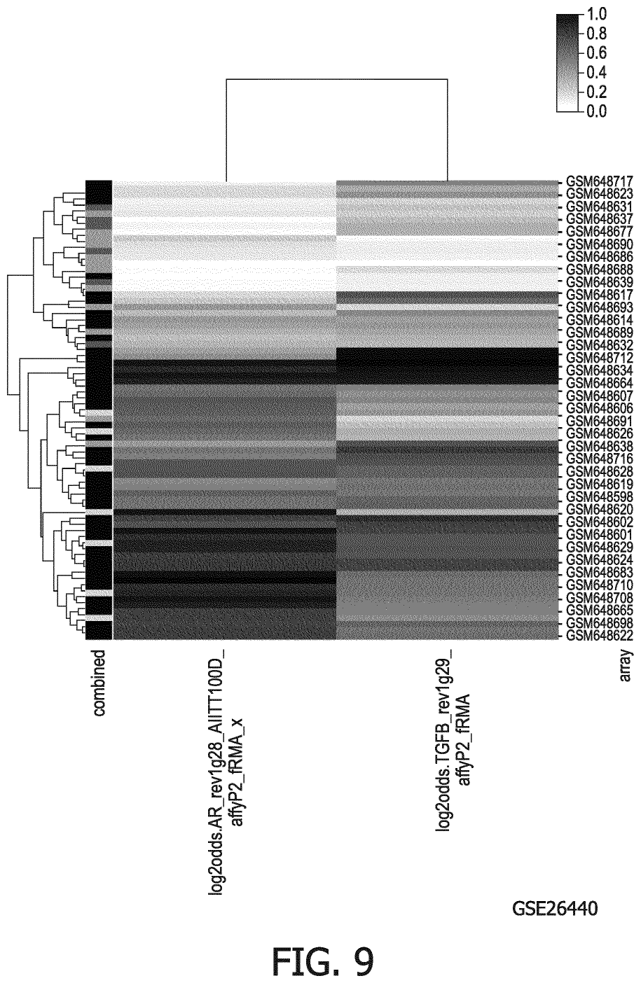

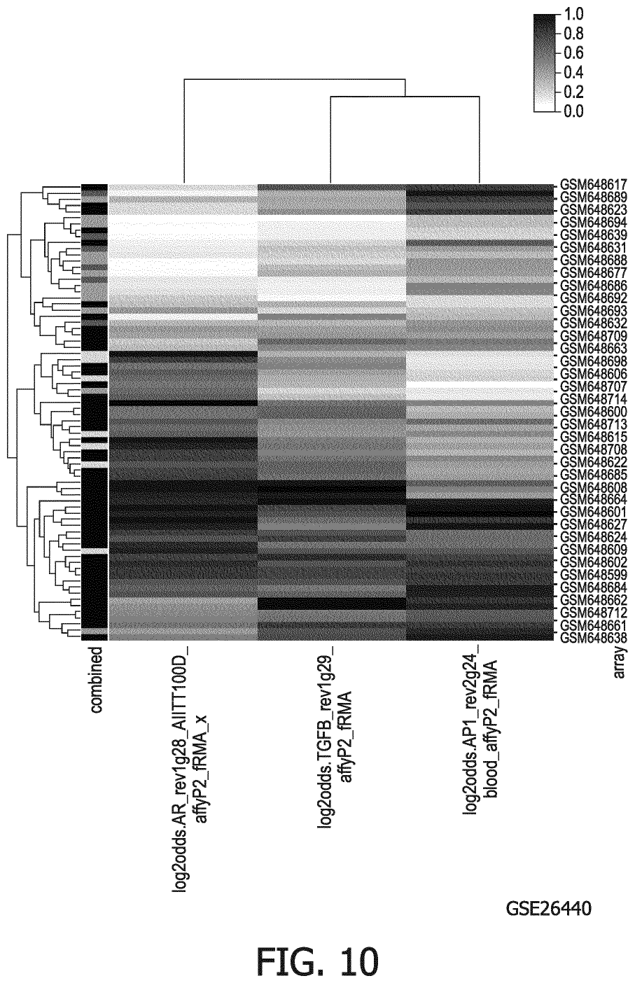

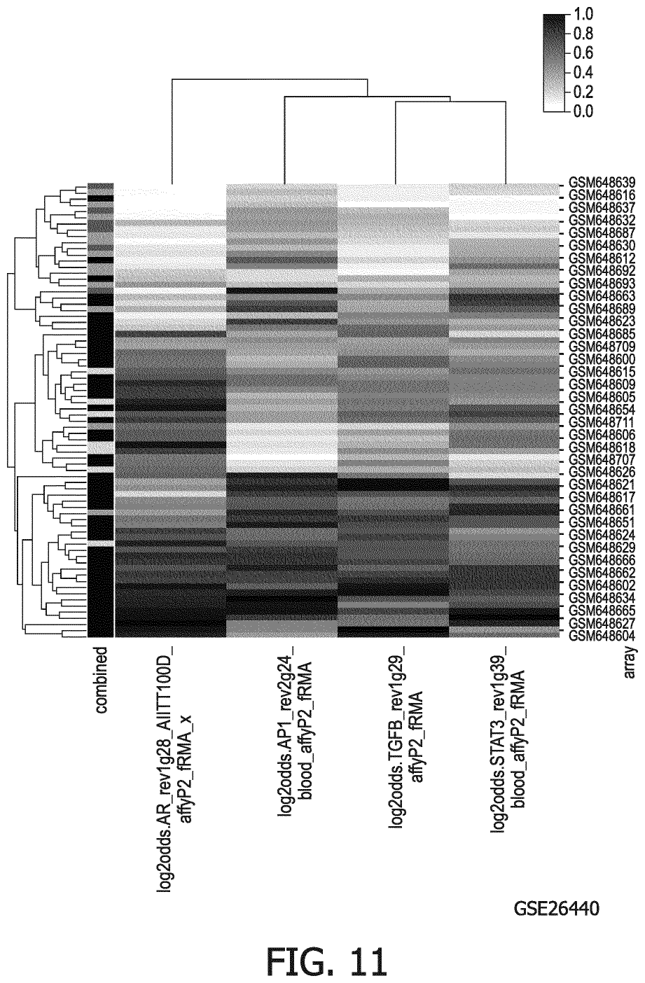

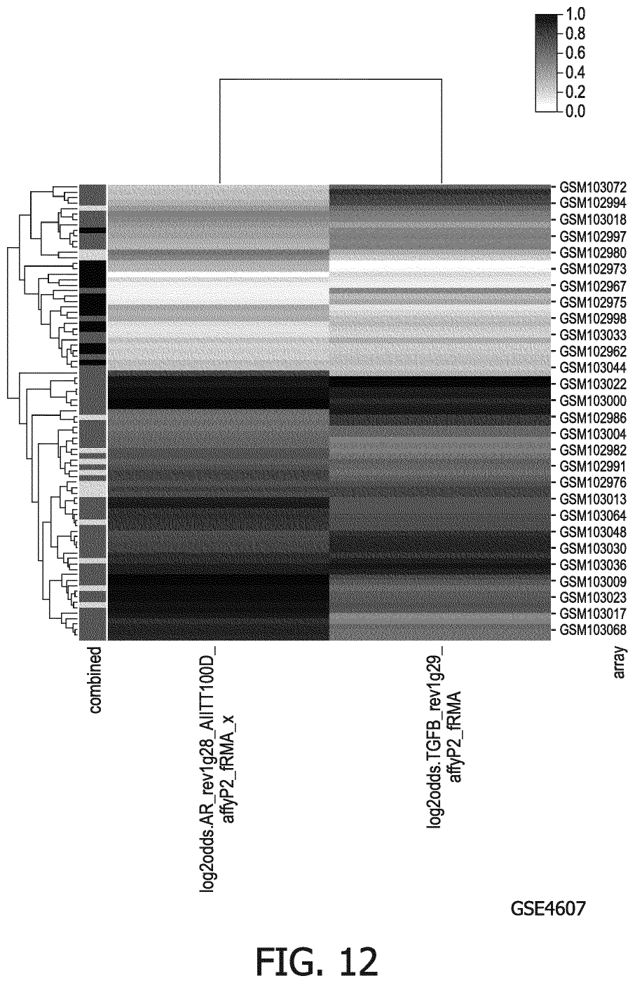

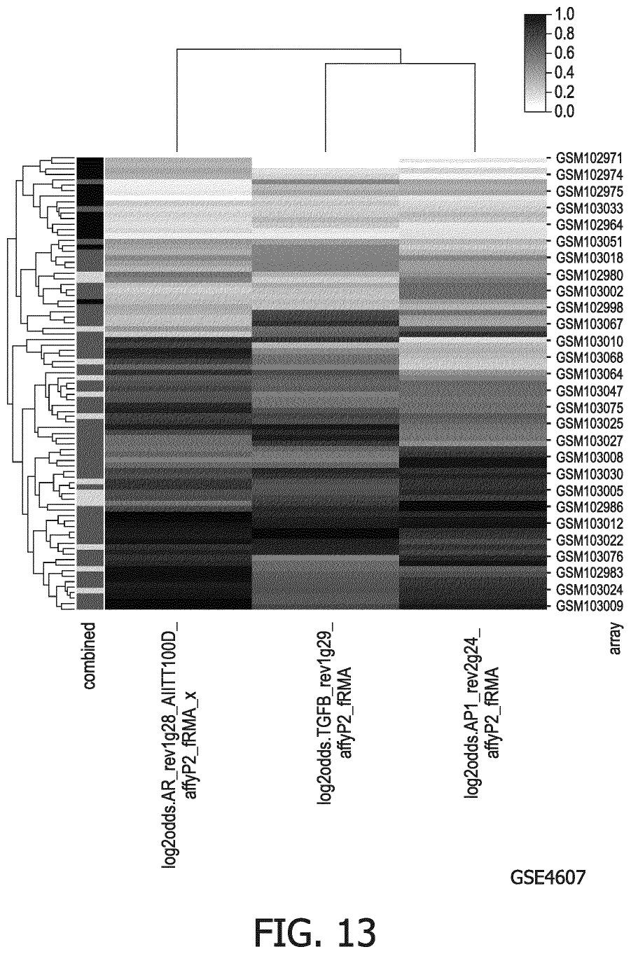

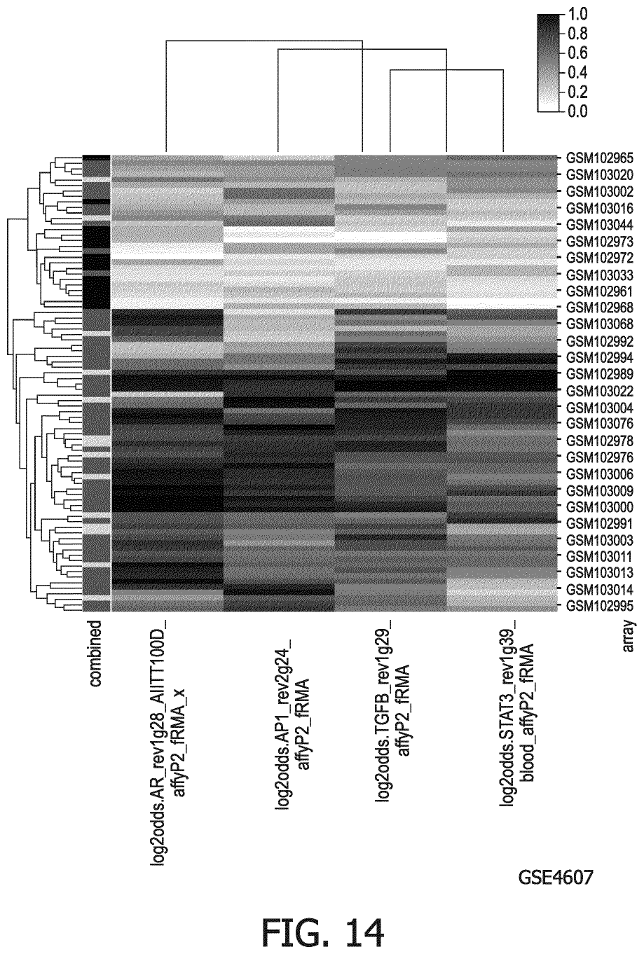

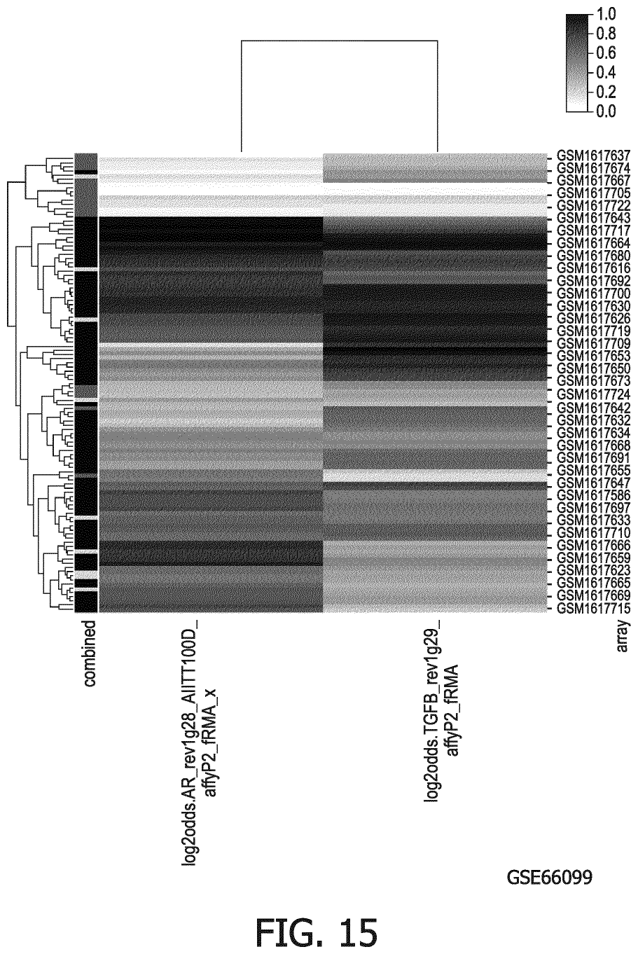

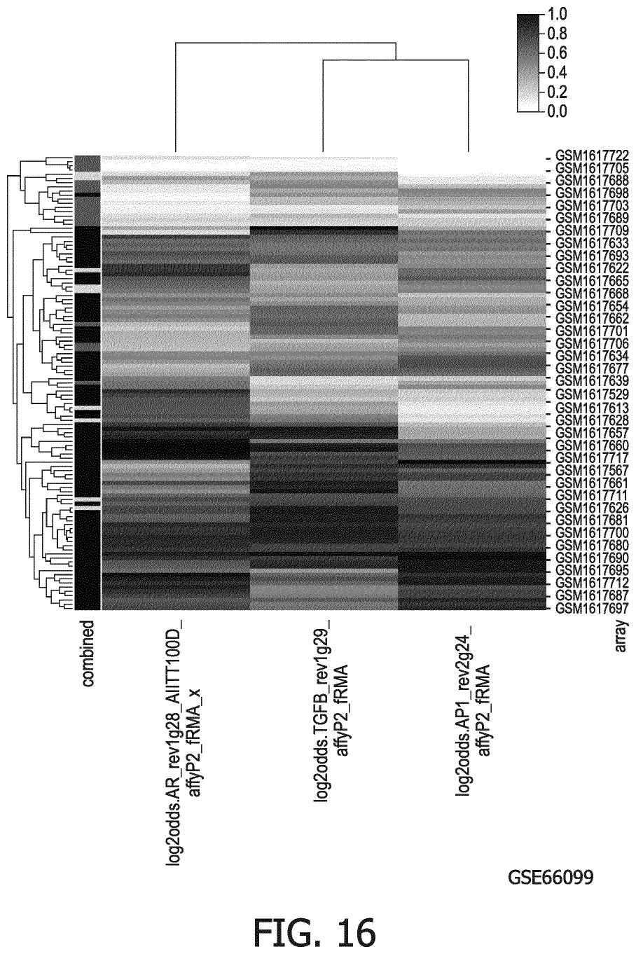

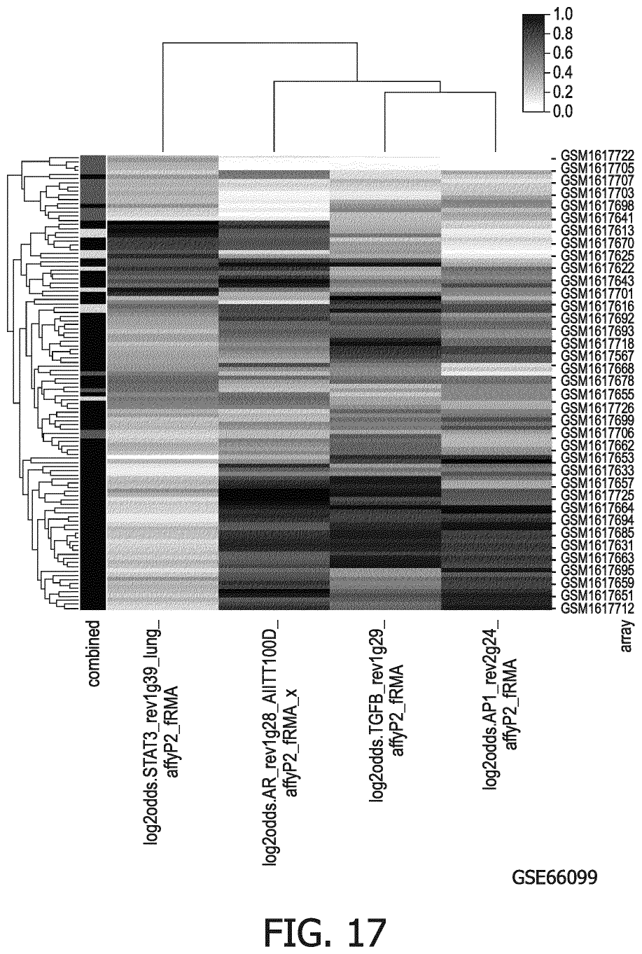

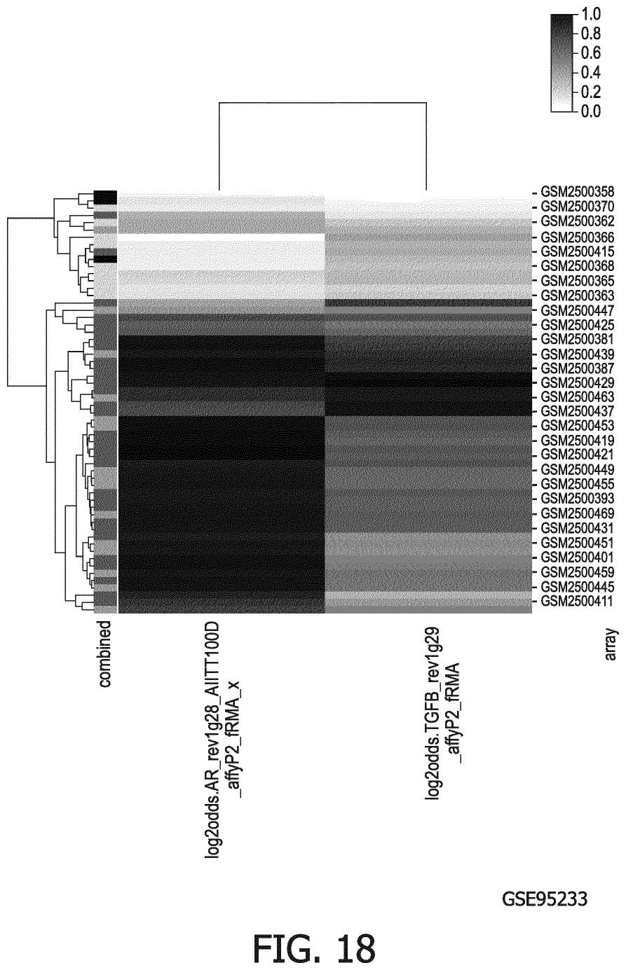

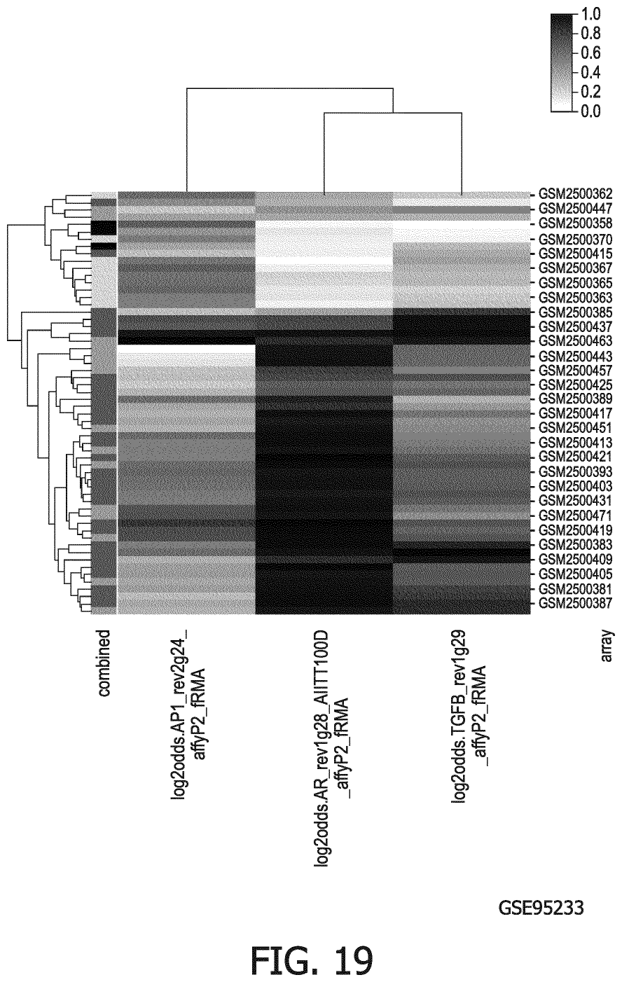

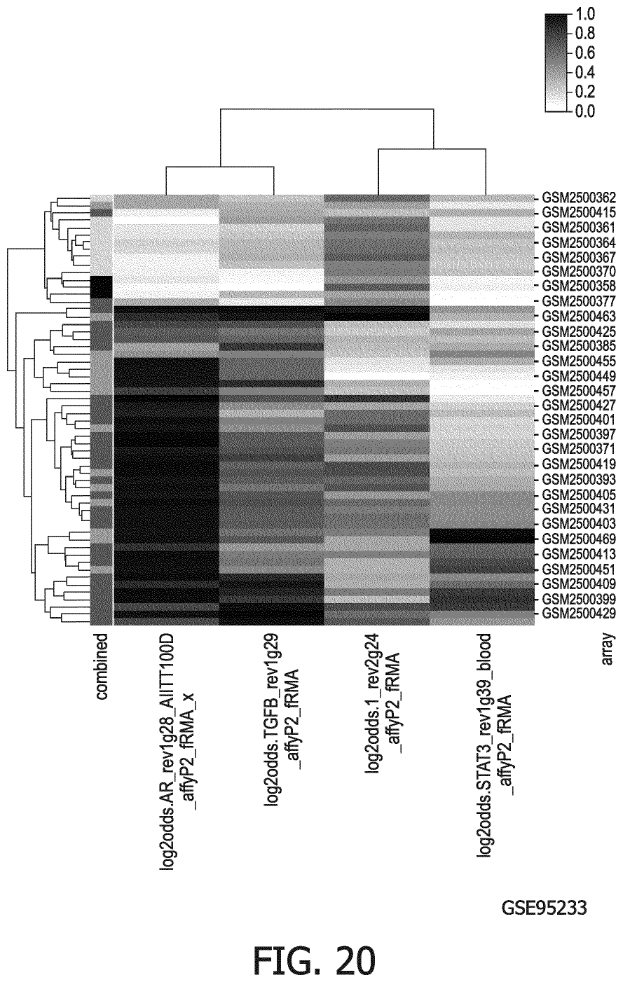

For analysis public Affymetrix U133P2.0 data were used from the GEO database (GSE26440, GSE4607, GSE66099, GSE95233, for more information about sample type and preparation see Table 1). Pathway analysis of datasets GSE26440, GSE4607, GSE66099 and GSE95233 showed significant differences in multiple pathways including AR and TGFBeta pathway activity using Mann-Whitney-Wilcoxon two-sided test between normal (healthy) control subjects and septic shock subjects (for pathways and p values see FIGS. 1-8).

Using a combination of significant pathways, we could identify/diagnose the sepsis subjects from the controls. Furthermore, based on the AR and or TGFBeta pathway activity a computational model was made to calculate a risk score with respect to the risk to die and survive from sepsis. With hierarchical clustering we identified samples which clustering near control/healthy people, which are more likely to survive.

TABLE 1
Sample type and preparation information per dataset.

| | Patients | | |
|---|---|---|---|
| | original in | | |
| GEO Dataset | dataset | Patients | Method |
| GSE26440 | 76 | Children <=10 | blood samples were obtained within 24 hours of |
| | (duplicates | years | initial presentation to the pediatric intensive |
| | removed for | | care unit with septic shock. |
| | analysis) | | Total RNA was isolated from whole blood samples |
| | | | using the PaxGene ™ blood RNA system. |
| GSE4607 | 83 | Children <10 | blood samples (for RNA and serum isolation) were |
| | | years | obtained within 24 hours of admission to the |
| | | | PICU, heretofore referred to as “Day 1” of septic |
| | | | shock. |
| | | | Total RNA was isolated from whole blood samples |
| | | | using the PaxGene ™ Blood RNA System |
| | | | (PreAnalytiX, Qiagen/Becton Dickson, Valencia, CA |
| | | | Patients meeting criteria for “sepsis” or “severe |
| | | | sepsis” were placed in the categories of SIRS and |
| | | | septic shock, respectively, for study purposes. |
| GSE66099 | 128 | Patients from | Consist of dataset; GSE4607, GSE8121, GSE9692, |
| (unique | (duplicates | 5 days to 18 | GSE13904, GSE26378, and GSE26440, data was |
| patients from | removed for | years old. | renormalized again. All datasets use the |
| GSE4607, | analysis) | | PaxGene ™ Blood RNA System and whole blood |
| GSE8121, | | | as input |
| GSE9692, | | | |
| GSE13904, | | | |
| GSE26378, | | | |
| and GSE26440) | | | |
| GSE95233 | 125 | 53-80 years | The first whole blood sample (EDTA tubes) was |
| | | | collected at the onset of shock (i.e., within 30 min |
| | | | after the beginning of vasoactive treatment, D 0) |
| | | | Total RNA was extracted with PAXgene ™ Blood |
| | | | RNA kit. |

### Example 2: Computational Models to Calculate a Risk Score

Furthermore, we were able to classify low, medium and high-risk sepsis subjects, with respect to the risk to die from sepsis. We used the computational model-based interpretation of multiple signaling pathway activity scores to classify the low, medium and high-risk sepsis subjects.

To construct a linear model for interpretation of pathway activity scores we assessed the pathway activities in healthy people, by calculating the average pathway activity with 1 and 2 Standard Deviation (SD). When a pathway activity falls outside the boundaries of 2SD of the normal healthy, we consider this an abnormally active pathway, which means in the model 1 point. Optionally another threshold, such as 3SD of the mean, can be used. Adding up the points generates a cumulative abnormal pathway activity score, which directly determines the likelihood of the risk.

Other computational models to calculate a risk score can be Bayesian models, centroid-based models etc.

### Example 3: Linear Model Using Calibration and Validation Set

For this, we used dataset GSE26440 as training set model and validated the model with dataset GSE4607. For both the AR and TGFBeta pathway 2SD above the mean pathway activity scores measured in the healthy control population was used for the classification model, this same value was then applied to the independent validation dataset GSE4607. When both AR and TGFBeta were 2SD higher than the control samples, the sepsis subjects were classified as high-risk (2 points). When either AR or TGFBeta were 2SD higher than the control, subjects were classified as medium-risk (1 point) and less than 2 SD difference was classified as low-risk (0 points). See table 2 for the determined means, standard deviations and 2SD top boundary for the pathways.

For the prognostic model, low, medium and high-risk groups are identified for subject stratification. In the medium group one of the pathways is upregulated whereas in the high group both pathways are upregulated.

In table 3 the performance of the prognostic model is shown. For the GSE 26440 dataset (n=76, non-survivor 10% (n=8), survivor 68% (n=51), Control 22% (n=17)) we could classify of the non survivors group, 3 as high risk, 5 as medium risk and 0 as low risk. For the validation set GSE 4607 (n=83, non-survivor 17% (n=14), survivor 65% (n=54) and control 18% (n=15)) we could classify of the non survivors group 10 as high, 2 as medium and 2 as low using the model described above. In addition, the combined pathway sum score of AR and TGFBeta can also be used for the prognostic marker, in which high risk is classified (1 point) as the combined AR and TGFBeta pathway score were 2SD higher than the control samples. When the combined AR and TGFBeta score were less than 2 SD difference compared to control, the samples are classified as low-risk (0 points) (data not shown).

For the other datasets (GSE66099, GSE95233 and GSE57064) we also see samples with a low AR and/or TGFBeta pathway activity. However, we lack survival data to prove that these subjects have a higher change to survive from sepsis.

TABLE 2
Mean values, standard deviations and 2SD top boundary of the
activity of AR, TGFbeta and combined based on GSE26440.

| | | Standard | 2SD Top |
|---|---|---|---|
| Pathway | Mean | deviation | boundary |
| AR | 18.0 | 2.6 | 23.3 |
| TGFBeta | 13.1 | 2.6 | 18.4 |
| AR + TGFBeta combined | 31.2 | 4.4 | 40.0 |

TABLE 3
Model for the classification of low, medium and high-risk sepsis subjects
that are likely to die. (n = number of samples). SD based on top
boundary using control group. Scoring <2SD of AR or TGFBeta −>
Low, >2 SD of only AR or TGFBeta −> Medium, >2 SD of AR and TGFBeta −> High.

| | Training | | Validation | |
|---|---|---|---|---|
| | GSE 26440 (n = 76, | | GSE 4607 (n = 83, | |
| | non survivor (8) | | non survivor 17% (14) | |
| | survivor (51) | | survivor 65% (54) | |
| Model | control (17)) | | control 18% (15)) | |
| (2SD of AR, | Prediction | | Prediction | |
| TGFBeta) | NS and S | | NS and S | |
| High risk | 38% (3/8) NS | non survivor (3) | 71% (10/14) NS | non survivor (10) |
| (AR + | 43% (22/51) S | survivor (22) | 57% (31/54) S | survivor (31) |
| TGFBeta high) | | control (0) | | control (0) |
| Medium risk | 62% (5/8) NS | non survivor (5) | 14% (2/14) NS | non survivor (2) |
| (AR or | 35% (18/51) S | survivor (18) | 28% (15/54) S | survivor (15) |
| TGFBeta high) | | control (2) | | control (0) |
| Low risk | 0% (0/8) NS | non survivor (0) | 14% (2/14) NS | non survivor (2) |
| (AR or | 22% (11/51) S | survivor (11) | 64% (9/14) S | survivor (9) |
| TGFBeta low) | | control (15) | | control (15) |

### Example 4: Linear Model 2—Using Top Boundaries Per Dataset

Due to the differences between tests and sample taking it is probably more specific to determine top boundaries per dataset. In table 4 the Mean values, standard deviations and SD top boundaries of the activity of AR of GSE26440 and GSE4607 are listed. In table 5, the above described linear model is used but in this example a 2 SD top boundary is used based on each separate dataset. All non-survivals are placed in the medium and high-risk group. The control samples are only located in the low group and could be used as a diagnostic marker.

TABLE 4
Mean values, standard deviations and SD top boundaries
of the activity of AR of GSE26440 and GSE4607

| | | Standard | 1SD Top | 2SD Top | 3SD Top |
|---|---|---|---|---|---|
| Dataset | Mean | deviation | boundary | boundary | boundary |
| GSE26440 | 18.0 | 2.6 | 20.6 | 23.3 | 25.9 |
| GSE4607 | 17.8 | 1.8 | 19.6 | 21.4 | 23.3 |

TABLE 5
Model for the classification of Low risk, medium risk and high-risk
sepsis subjects that are likely to die based on AR + TGFBeta. SD
based on top boundary using control group. Scoring <2SD of AR or
TGFBeta −> Low, >2 SD of only AR or TGFBeta −> Medium, >2
SD of AR and TGFBeta −> High. n = number of samples).

| | GSE 26440 (n = 76, | | GSE 4607 (n = 83, | |
|---|---|---|---|---|
| | non survivor (8) | | non survivor 17% (14) | |
| | survivor (51) | | survivor 65% (54) | |
| | control (17)) | | control 18% (15)) | |
| Model | Prediction | | Prediction | |
| 2 × SD | NS and S | | NS and S | |
| High risk | 38% (3/8) NS | non survivor (3) | 79% (11/14) NS | non survivor (11) |
| (AR + | 43% (22/51) S | survivor (22) | 69% (37/54) S | survivor (37) |
| TGFBeta high) | | control (0) | | control (0) |
| Medium risk | 62% (5/8) NS | non survivor (5) | 21% (3/14) NS | non survivor (3) |
| (AR or | 35% (18/51) S | survivor (18) | 15% (8/54) S | survivor (8) |
| TGFBeta high) | | control (2) | | control (0) |
| Low risk | 0% (0/8) NS | non survivor (0) | 0% (0/14) NS | non survivor (0) |
| (AR or | 22% (11/51) S | survivor (11) | 17% (9/54) S | survivor (9) |
| TGFBeta low) | | control (15) | | control (15) |

### Example 5: Linear Model 3—Using Only AR Pathway and Top Boundaries Per Dataset

The same principle as described above is used in this example, only a 2DS top boundary is used and the model is based on only the AR pathway. The risk groups are in this case low or high risk of (dying from sepsis. The performance of the model can be found in Table 6. The control samples are only located in the low group and could be used as a diagnostic marker.

TABLE 6
Prognostic model for the classification of Low and high-risk
sepsis subjects that are likely to die and survive. (n =
number of samples). SD based on top boundary using control
group. Scoring <2SD of AR −> Low, >2 SD of AR −> High.

| | GSE 26440 (n = 76, | | GSE 4607 (n = 83, | |
|---|---|---|---|---|
| | non survivor (8) | | non survivor 17% (14) | |
| | survivor (51) | | survivor 65% (54) | |
| | control (17)) | | control 18% (15)) | |
| Model | Prediction | | Prediction | |
| (2SD of AR) | NS and S | | NS and S | |
| High (AR) | 100% (8/8) NS | non survivor (8) | 93% (13/14) NS | non survivor (13) |
| | 61% (31/51) S | survivor (31) | 72% (39/54) S | survivor (39) |
| | | control (1) | | control (0) |
| Low (AR) | 0% (0/8) NS | non survivor (0) | 7% (1/14) NS | non survivor (1) |
| | 39% (20/51) S | survivor (20) | 28% (15/54) S | survivor (15) |
| | | control (16) | | control (15) |

### Example 6: Clustering Methods

We used hierarchical clustering (seaborn clustermap) to determine if it was possible to classify subjects based on their pathway activity. We selected the significant models between the control group and sepsis groups for the pathways AR, TGFBeta, JAK-STAT3 and MAPK-AP1.

For dataset GSE4607, several sepsis samples are clustered near the control group (orange). These subjects probably have a higher chance on survival. These samples are also clustered in the low risk group in the model described above which was based on the AR and TGFBeta. However, a clear distinction between the Septic Shock survivor and non survivors is not shown.

### Example 7: Cell Stimulation with H. Pylori

To investigate whether bacteria or bacterial products could induce the same pathway activities as observed in patients with sepsis, in vitro experiments were performed in which either bacteria or the bacterial product LPS was added to monocytic cells from a cell line, as a model system for monocytes in blood.

Porphyromonas gingivalis (ATCC, 33277) bacteria were cultured in an anaerobic culture hood using dehydrated HBI (Oxoid, CM1032) using manufactures instructions.

THP-1 cells were cultured in 6-wells plates with a density of 4×105 cells/well for 48 hours. After 48-hour cell seeding, cells were washed with PBS and treated with either direct bacteria of 1:100 MOI or using the 20% ‘supernatant’ of the bacterial culture at 37° C. 5% CO2 for 4 hours. Cells were exposed to bacteria with a MOI of 1:100, the 20% ‘supernatant’ was prepared by filtering the overnight bacteria culture was fusing 0.2 uM filter to remove whole bacteria and hereafter diluted in the cell culture media to a 20% concentration. After the 48-hour cell seeding, cells were washed with PBS and treated with either direct bacteria of 1:100 MOI or using the 20% ‘supernatant’ of the bacterial culture at 37° C. 5% CO2 for 4 hours. Hereafter, cells were washed with PBS and lysed in RNeasy mini kit lysis buffer (Qiagen, Cat No./ID: 74104) and stored at −80° C. until further processing. RNA was extracted using the RNeasy mini kit (Qiagen, 74104). qPCR was performed using the Philips Research OncoSignal platform.

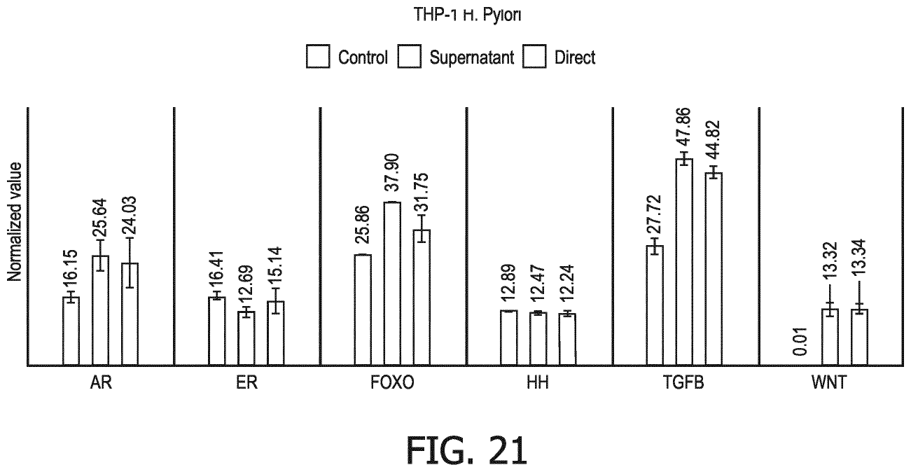

To determine whether the Helicobacter bacteria have a bacteria-specific effect on the pathways activities of THP-1 cells, qRT-PCR was performed. Cells were either treated with direct bacteria or the growth media of the bacteria culture. As shown in FIG. 21, the pathway activity was increased for the AR, FOXO, TGFbeta and the WNT pathway. In the sepsis samples however, we did not detect a significant difference in the FOXO and WNT pathways, which could be due to the fact that monocytes only consist of 4-8% of the blood composition, and other blood cell types also play an important role.

### Example 8: Cell Stimulation with LPS

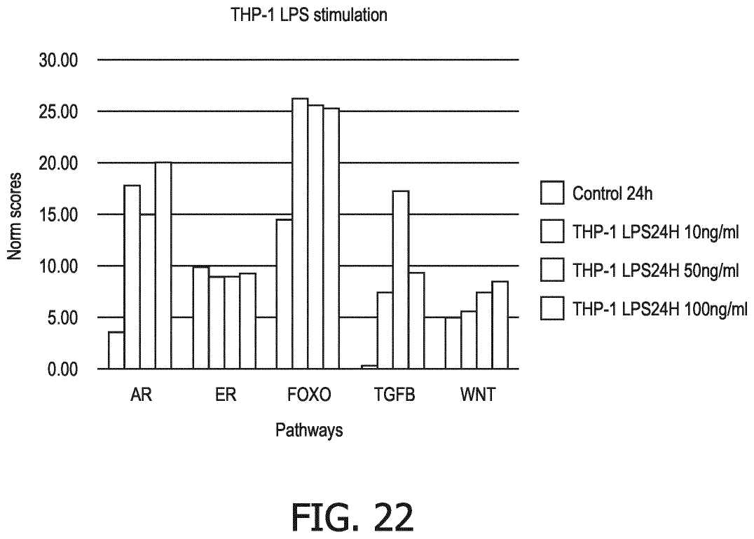

To study the inflammation process we stimulated the monocytic THP-1 cells (ATCC® TIB-202™) using 3 different concentrations of LPS originated from E. coli (0 ng/ml, 10 ng/ml, 50 ng/ml and 100 ng/ml) into culture medium (DMEM, supplemented with 10% FBS, 1% glutamax and 1% pen strep at 37° C. 5% CO2.) of THP-1 for 24 hours. After stimulation cells were harvested and RNA was extracted using the RNeasy mini kit (Qiagen, 74104). qPCR was performed using the Philips Research OncoSignal platform. As shown in FIG. 22, the pathway activity was increased for the pathways AR, FOXO, TGFBeta and WNT in the LPS stimulated cells. The activation of the AR and TGFBeta pathways was also seen in sepsis samples confirms the role of these pathways in inflammation. In the sepsis samples however, we did not detect a significant difference in the FOXO and WNT pathways, which could be due to the fact that monocytes only consist of 4-8% of the blood composition, and other blood cell types also play an important role.

### Example 9: Validation of Subsets of Target Genes

To validate whether subsets of pathway target genes (e.g. three target genes selected from the total) are still predictive, random selections of N genes were made for the AR and the TGFbeta cellular signaling pathway target genes to evaluate the chance that a random selection of N genes from the total list is predictive. In order to do this, the individual target genes of the AR and TGFbeta cellular signaling pathway were ranked based on their relative contribution to the pathway score (T) as described below. For different thresholds of T (where T=0 corresponds to the entire gene set, and subsequent higher values for T correspond to more stringent selection) N genes were selected randomly 1000 times and a score was calculated as indicated below using a very simple linear model. The computation was performed for N values ranging from 1 to 6 on datasets GSE26440, GSE4607 and GSE66099 combined (set 1), or GSE95233 (set 2), or GSE57065 (set 3). Further the calculations were performed on the AR target genes, the TGFbeta target genes and pooled AR and TGFbeta target genes.

The protocol was performed as follows:
1. Take the list of genes corresponding to the pathways of interest, and take their probesets.
2. Per gene, take the probeset with maximum absolute correlation with the pathway score it contributes to (based on all sepsis and control samples; a gene may be involved in multiple pathways).
3. Select a candidate gene list by taking all genes with their absolute probeset-pathway correlation above a threshold T.
4. Repeatedly (1000 times), choose a random sub-list of N genes from the candidate gene list.
5. Make a simple linear classifier with those N genes by assigning them a weight +1 or −1 depending on the sign of their probeset-pathway correlation.
6. Apply that linear classifier on all samples to calculate a score.
7. For each test set, either GSE26440, GSE4607 and GSE66099 combined, or GSE95233, or GSE57065:
a. determine a mean and standard deviation of the score on the normal samples
b. calculate a threshold by taking the mean plus two times the standard deviation
c. determine the fraction of sepsis samples above the threshold, the fraction of sepsis non-survivors (if given) above the threshold, and for a check also the fraction of normal samples above the threshold
8. Make a box-plot distribution of the determined fractions over the 1000 random draws.

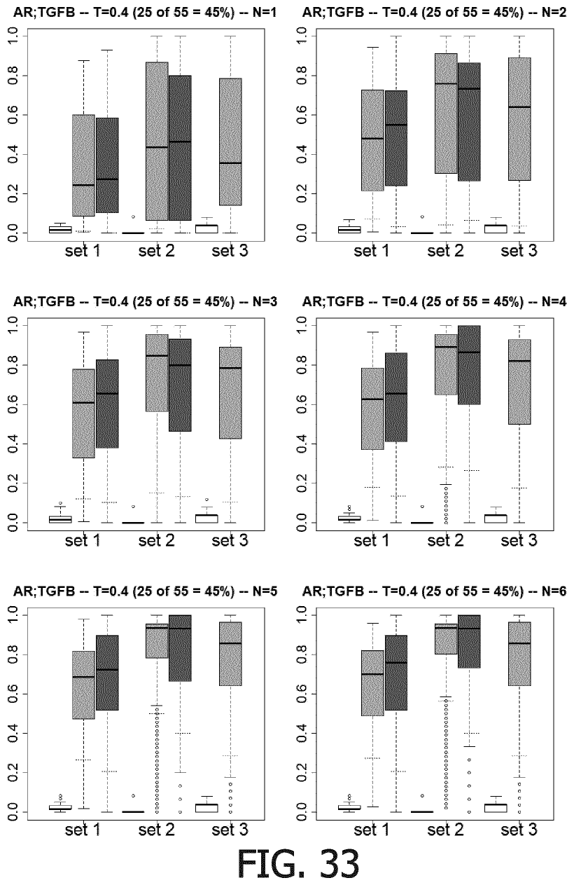

For an example where we consider the AR and TGFB pathways, a correlation threshold T=0.4, augmented with manually selected genes (FIG. 33), and random sets of N=3 genes, the results on the combination test set GSE26440, GSE4607 and GSE66099 shows:
the median fraction (thick line in the box) of detected sepsis samples is about 0.60, meaning that half of the random lists give a sensitivity of 60% or higher,
the 25th percentile (lower boundary of the box) of detected sepsis samples is about 0.32, meaning that three quarters of the random lists give a sensitivity of 32% or higher,
the 10th percentile (small dotted horizontal line) of detected sepsis samples is about 0.12, meaning that 90% of the random lists give a sensitivity of 12% or higher,
the specificity is at about 97.5% by the choice of the threshold (mean+2 stdev), and this is confirmed by the low fractions observed for the normal samples.

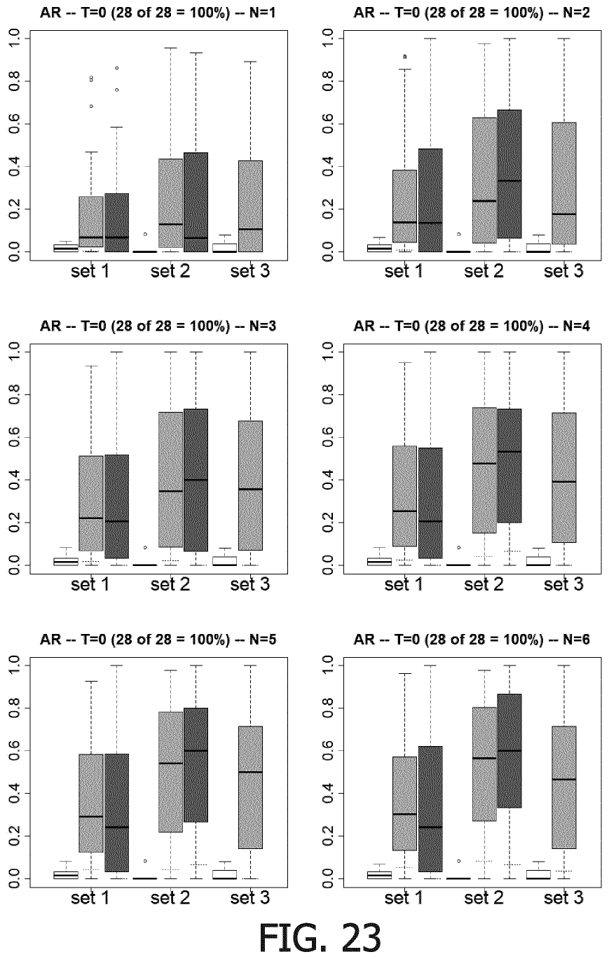

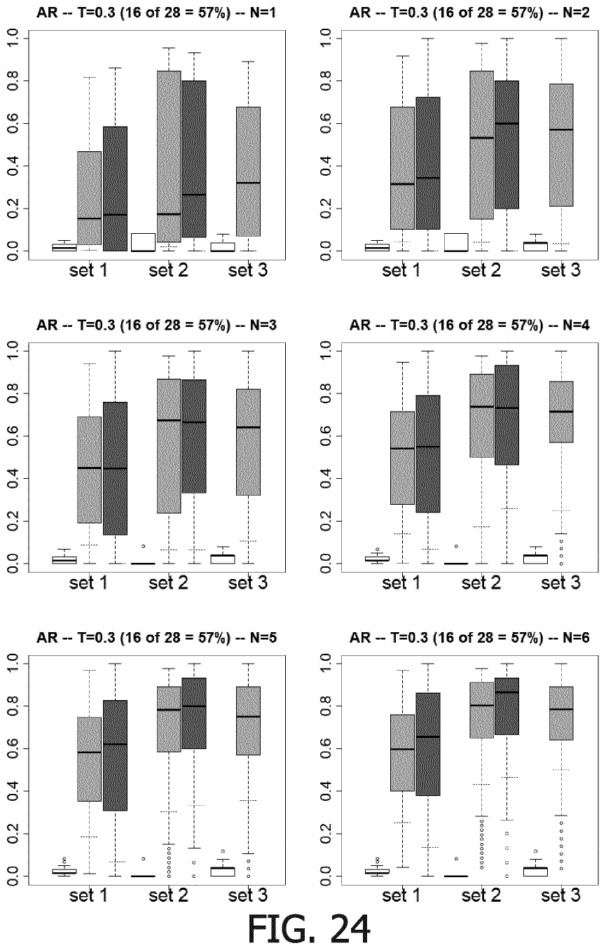

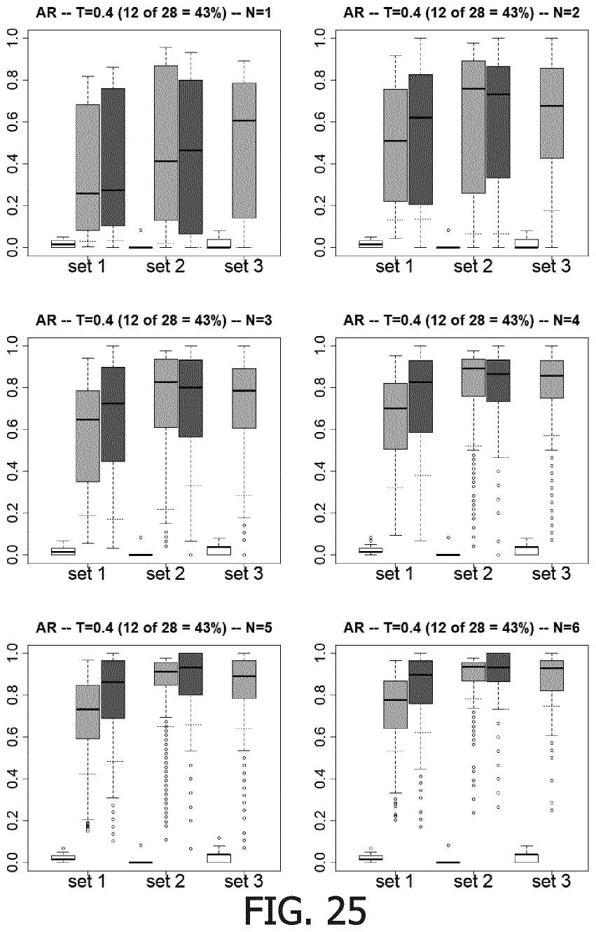

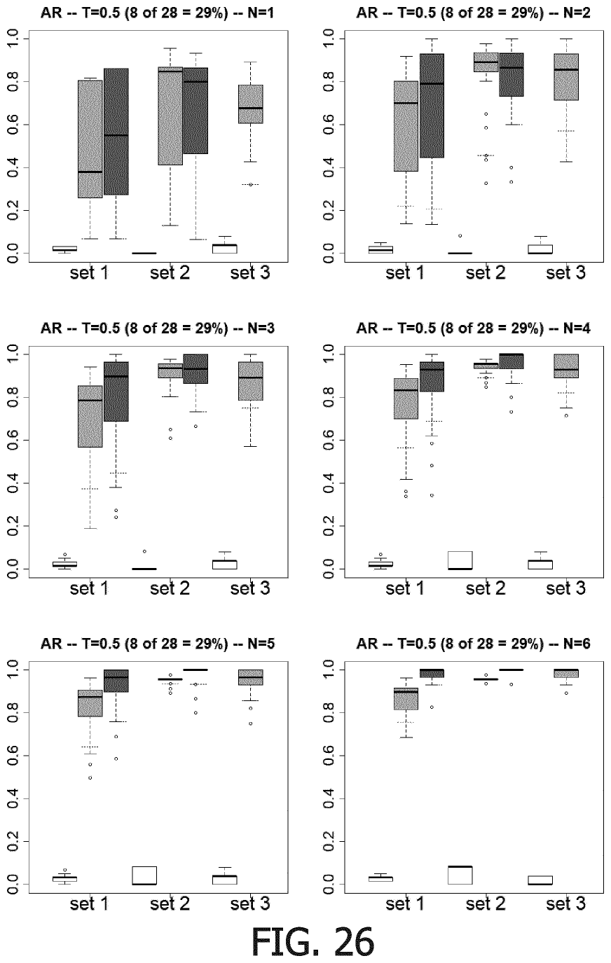

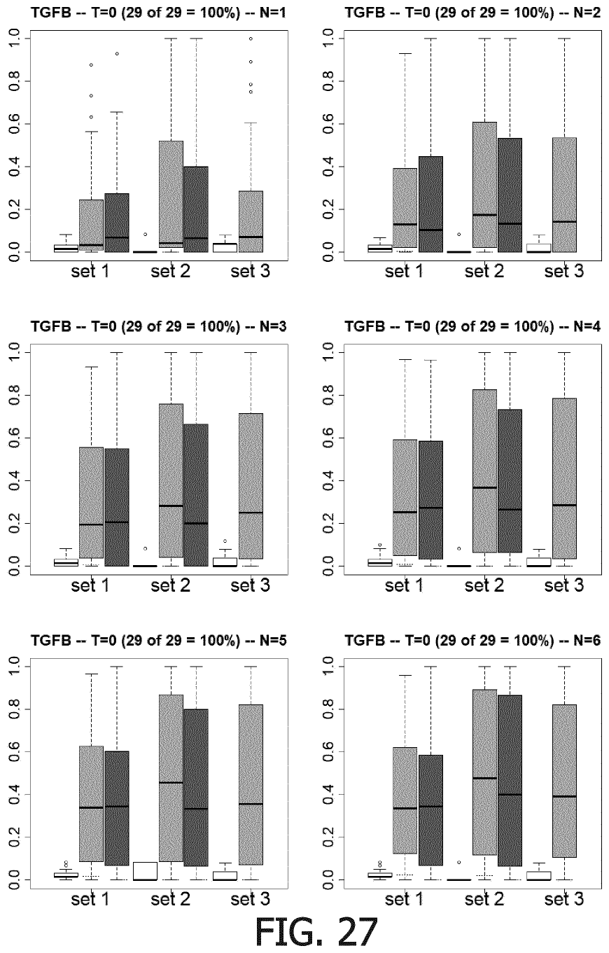

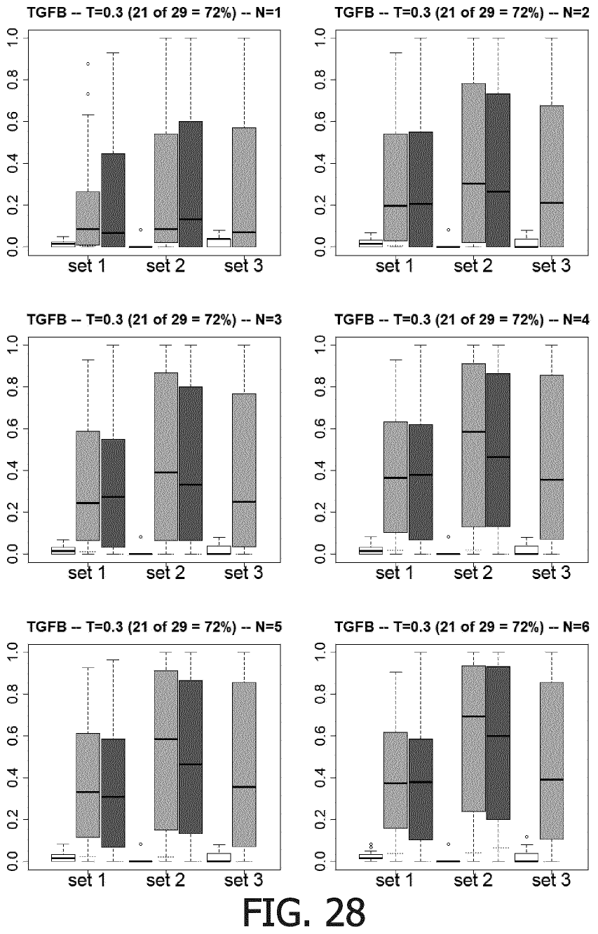

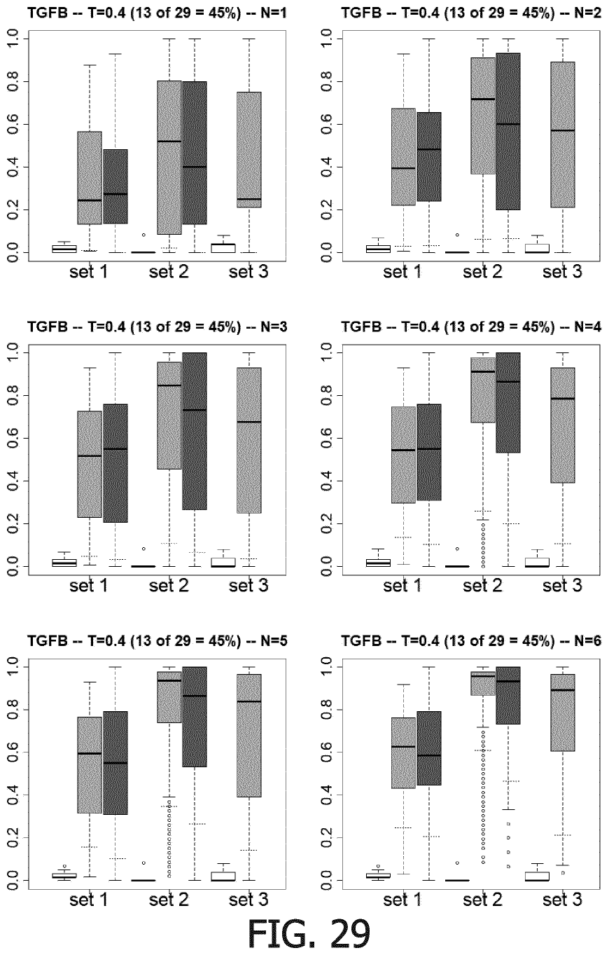

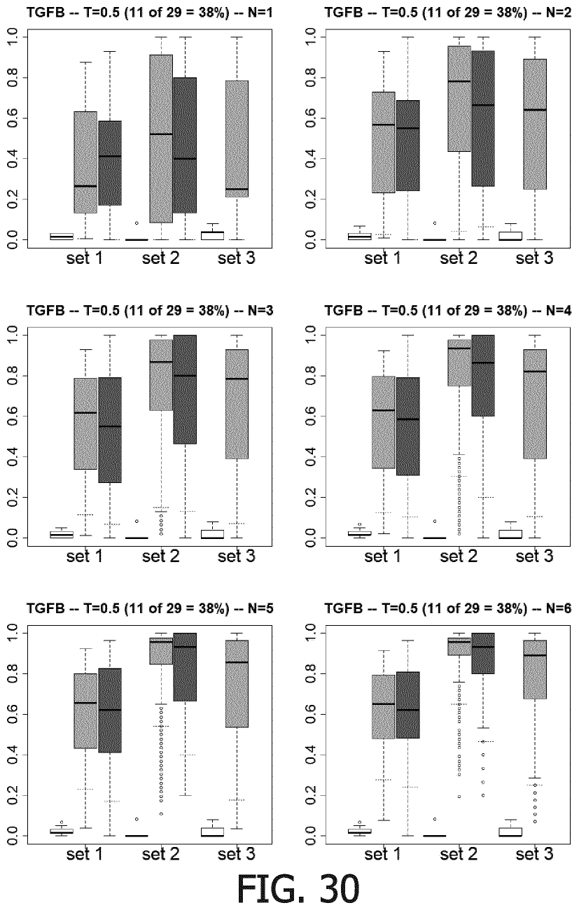

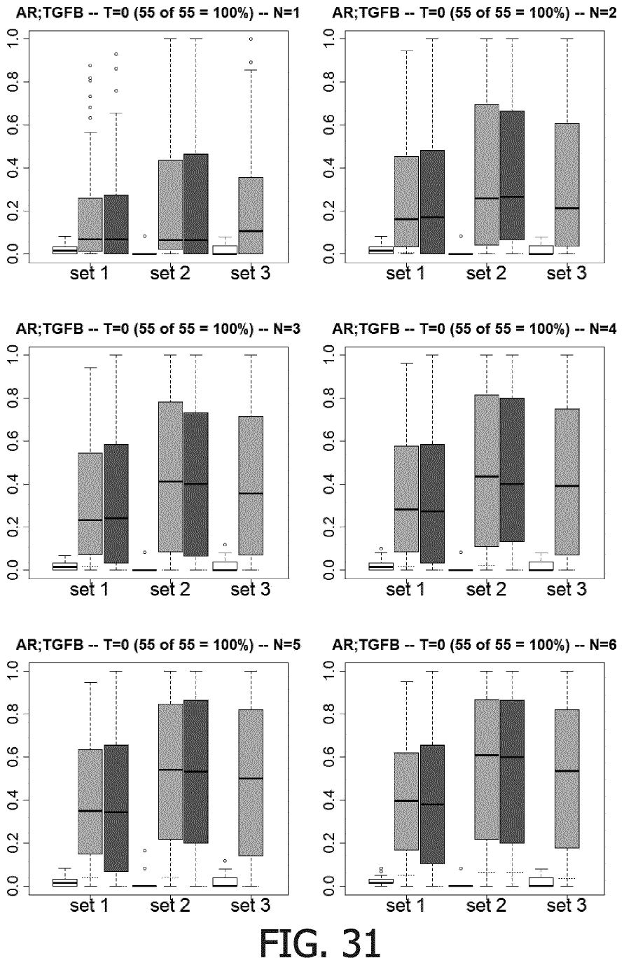

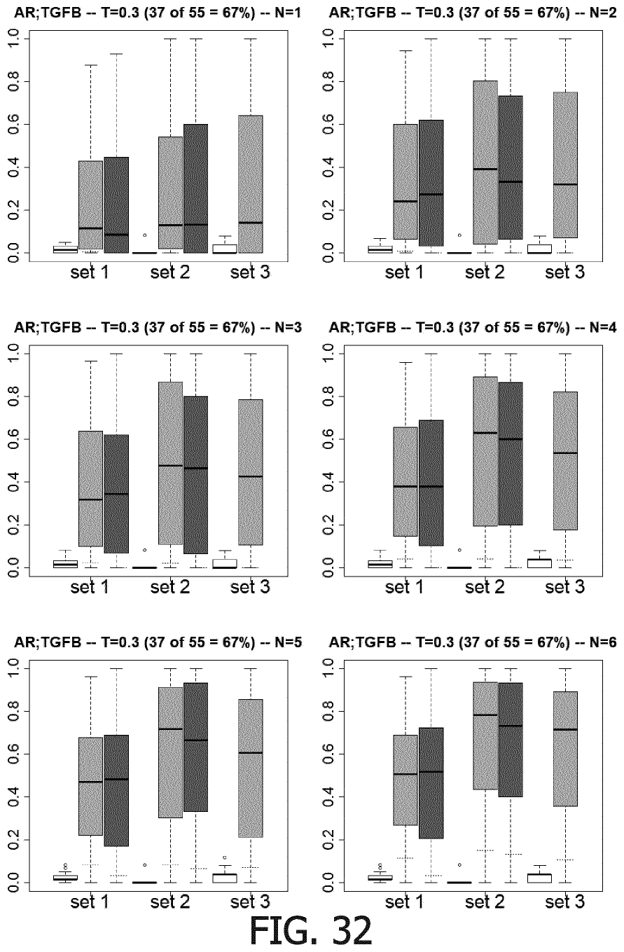

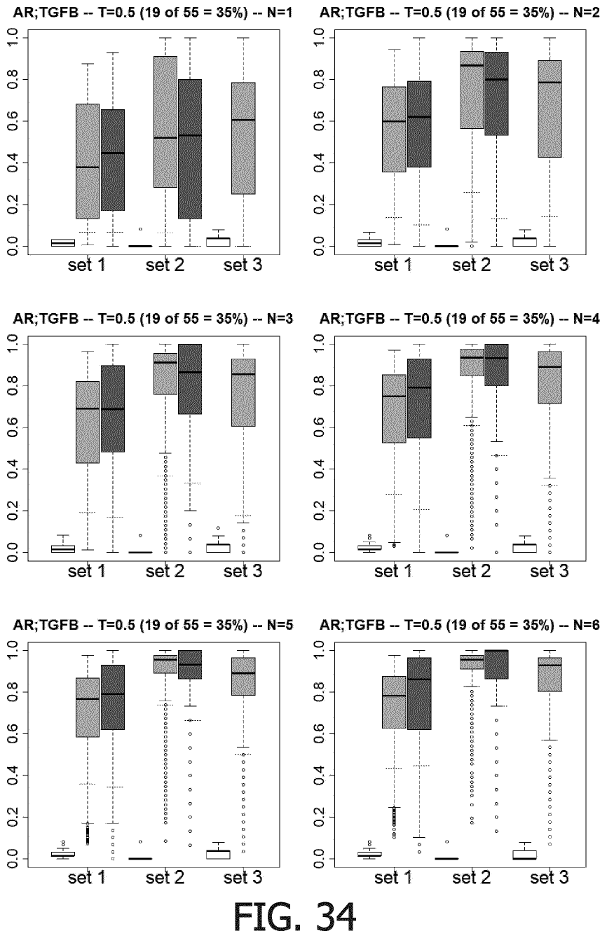

The boxplots resulting from these subsets are depicted in FIGS. 23 to 34. From these datasets it can be concluded that depending on the dataset used and the selection criteria for target genes, as few as 1 target gene may be sufficient to distinguish between blood samples obtained from septic and non-septic subjects, but in all case a random selection at least three genes results in a set of genes with high specificity and a desirable sensitivity. Therefore it is concluded that a minimum of three target genes of the AR cellular signaling pathway, the TGFbeta cellular signaling pathway, or the pooled target genes from the AR and the TGFbeta cellular signaling pathway (as defined herein) suffice to diagnose a subject with sepsis.

From these data it can be concluded that a sepsis diagnosis can reliably be made based on three gene expression levels selected from the various sets of genes presented here. Although the respective sets of genes were successfully identified using the pathway models, this example demonstrates it is not necessary to use the pathway models in the diagnosis, and that diagnosis can be done purely based on the expression levels alone.

The genesets used in the analysis are as follows (the symbol in front of the gene name indicating positive or negative correlation):
AR−T=0
AR: +ABCC4, +APP, −AR, −CDKN1A, −CREB3L4, +DHCR24, +EAF2, +ELL2, +FGF8, +FKBP5, −GUCY1A3, +IGF1, −KLK2, −KLK3, +LCP1, −LRIG1, +NDRG1, −NKX3_1, −NTS, −PLAU, −PMEPA1, −PPAP2A, −PRKACB, +PTPN1, +SGK1, −TACC2, −TMPRSS2, −UGT2B15
AR−T=0.3
AR: −AR, −CREB3L4, +DHCR24, +EAF2, +ELL2, +FKBP5, −GUCY1A3, +IGF1, −KLK3, +LCP1, −LRIG1, +NDRG1, −NKX3_1, −PMEPA1, −PRKACB, −TMPRSS2 AR−T=0.4
AR: −AR, −CREB3L4, +DHCR24, +EAF2, +ELL2, +FKBP5, +LCP1, −LRIG1, +NDRG1, −PMEPA1, −PRKACB, −TMPRSS2
AR−T=0.5
AR: +DHCR24, +EAF2, +ELL2, +FKBP5, +LCP1, −PMEPA1, −PRKACB
TGFB−T=0
TGFB: −ANGPTL4, +CDC42EP3, −CDKN1A, +CDKN2B, +CTGF, +GADD45A, +GADD45B, +HMGA2, +ID1, +IL11, +INPP5D, +JUNB, −MMP2, +MMP9, −NKX2_5, −OVOL1, −PDGFB, +PTHLH, +SERPINE1, +SGK1, +SKIL, +SMAD4, −SMAD5, +SMAD6, −SMAD7, −SNAI1, +SNAI2, +TIMP1 and +VEGFA
TGFB−T=0.3
TGFB: +CDC42EP3, +GADD45A, +GADD45B, +HMGA2, +ID1, +IL11, +INPP5D, +JUNB, −MMP2, +MMP9, −NKX2_5, −OVOL1, −PDGFB, +PTHLH, +SGK1, +SKIL, +SMAD4, −SMAD5, +SMAD6, +TIMP1, +VEGFA
TGFB−T=0.4
TGFB: +CDC42EP3, +GADD45A, +GADD45B, +ID1, +JUNB, +MMP9, −PDGFB, +SGK1, +SKIL, −SMAD5, +SMAD6, +TIMP1, +VEGFA
TGFB−T=0.5
TGFB: +CDC42EP3, +GADD45A, +GADD45B, +ID1, +JUNB, +MMP9, −PDGFB, −+SGK1, −SMAD5, +TIMP1, +VEGFA
AR; TGFB−T=0
AR: +ABCC4, +APP, −AR, −CDKN1A, −CREB3L4, +DHCR24, +EAF2, +ELL2, +FGF8, +FKBP5, −GUCY1A3, +IGF1, −KLK2, −KLK3, +LCP1, −LRIG1, +NDRG1, −NKX3_1, −NTS, −PLAU, −PMEPA1, −PPAP2A, −PRKACB, +PTPN1, +SGK1, −TACC2, −TMPRSS2, −UGT2B15
TGFB: −ANGPTL4, +CDC42EP3, −CDKN1A, +CDKN2B, +CTGF, +GADD45A, +GADD45B, +HMGA2, +ID1, +IL11, +INPP5D, +JUNB, −MMP2, +MMP9, −NKX2_5, −OVOL1, −PDGFB, +PTHLH, +SERPINE1, +SGK1, +SKIL, +SMAD4, −SMAD5, +SMAD6, −SMAD7, −SNAI1, +SNAI2, +TIMP1 and +VEGFA
AR; TGFB−T=0.3
AR: −AR, −CREB3L4, +DHCR24, +EAF2, +ELL2, +FKBP5, −GUCY1A3, +IGF1, −KLK3, +LCP1, −LRIG1, +NDRG1, −NKX3_1, −PMEPA1, −PRKACB, +SGK1, −TMPRSS2
TGFB: +CDC42EP3, +GADD45A, +GADD45B, +HMGA2, +ID1, +IL11, +1NPP5D, +JUNB, −MMP2, +MMP9, −NKX2_5, −OVOL1, −PDGFB, +PTHLH, +SGK1, +SKIL, +SMAD4, −SMAD5, +SMAD6, +TIMP1, +VEGFA
AR; TGFB−T=0.4
AR: −AR, −CREB3L4, +DHCR24, +EAF2, +ELL2, +FKBP5, +LCP1, −LRIG1, +NDRG1, −PMEPA1, −PRKACB, +SGK1, −TMPRSS2
TGFB: +CDC42EP3, +GADD45A, +GADD45B, +ID1, +JUNB, +MMP9, −PDGFB, +SGK1, +SKIL, −SMAD5, +SMAD6, +TIMP1, +VEGFA
AR; TGFB−T=0.5
AR: +DHCR24, +EAF2, +ELL2, +FKBP5, +LCP1, −LRIG1, −PMEPA1, −PRKACB, +SGK1 TGFB: +CDC42EP3, +GADD45A, +GADD45B, +ID1, +JUNB, +MMP9, −PDGFB, +SGK1, −SMAD5, +TIMP1, +VEGFA

### Example 10: Validation of AR Inhibitors as a Treatment Option for Sepsis

Based on the above described data it was theorized that sepsis may be treated, or at least its symptoms may be alleviated, by administering an AR cellular signaling pathway inhibitor. As can be deducted from Examples 7 and 8, and FIGS. 21 and 22, AR and TGFbeta cellular signaling pathway activities are increased upon stimulation with H. pylori supernatant or LPS in monocytes (THP-1 cells). To confirm this hypothesis, the applicant used this model system to predict a medical outcome of an AR pathway inhibitor for treating sepsis.

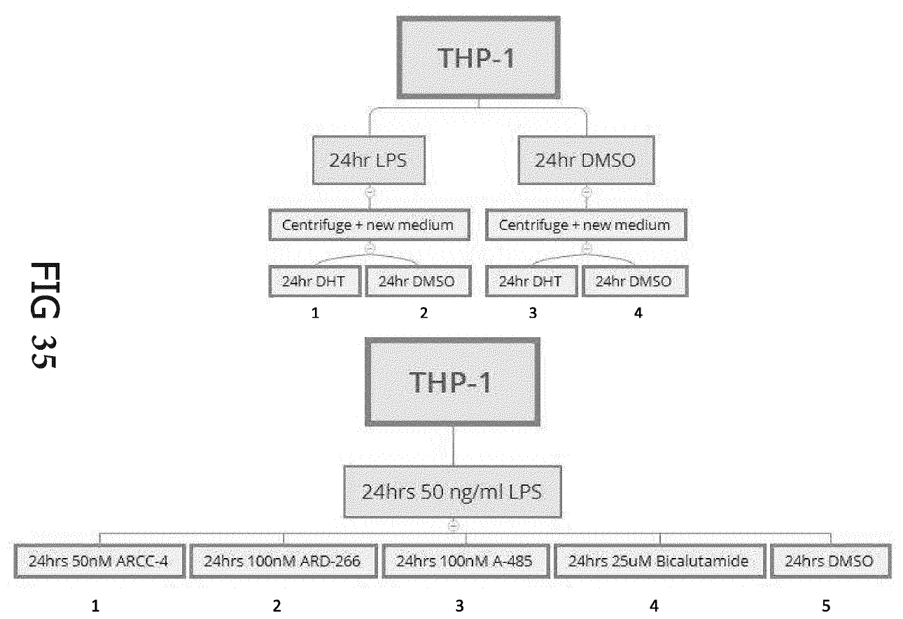

FIG. 35 describes the experimental set-up used. In brief, monocyte cells (THP-1) are cultured for 24 hours with or without LPS, after which the medium is changed and both conditions are subsequently cultured with or without DHT. Both LPS and DHT are anticipated to activate the AR cellular signaling pathway. In a parallel experiment, THP-1 cells are first cultured for 24 hours with LPS, after which the medium is changed and the cells are cultured with one of ARCC-4, ARD-266, A-458 and bicalutamide.

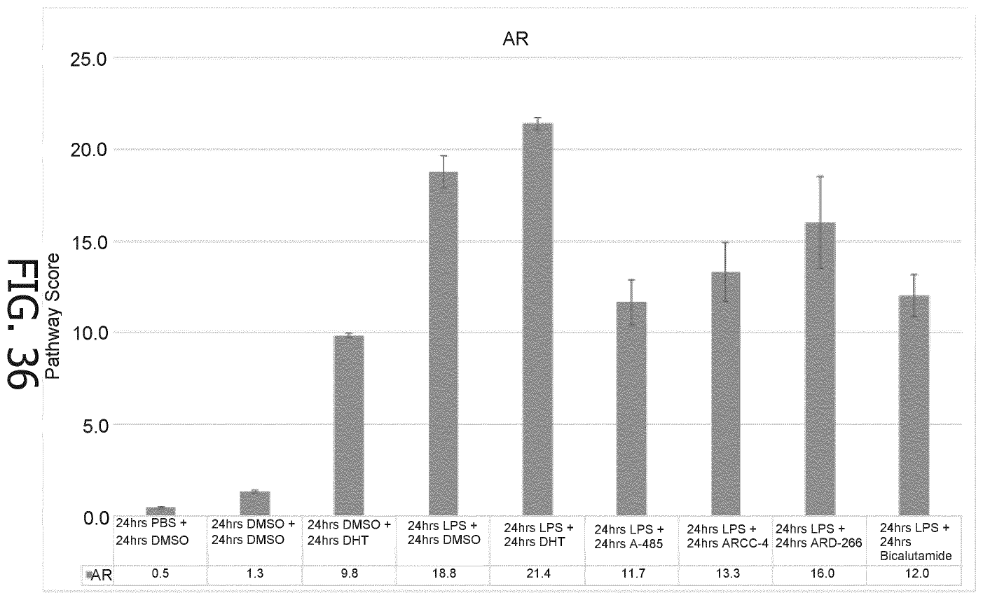

All experimental conditions were subjected to cellular signaling pathways analysis. The measured ER, AR, HH, and TGFbeta cellular signaling pathway activities are shown in FIGS. 36 to 39 respectively. FIG. 36 demonstrates that LPS or DHT increase AR cellular signaling pathway activity in monocytes, and that appears to be a small additive effect. Further, FIG. 36 demonstrates that AR activity induced by LPS can be at least partially reverted to baseline levels by addition of an AR pathway inhibitor.

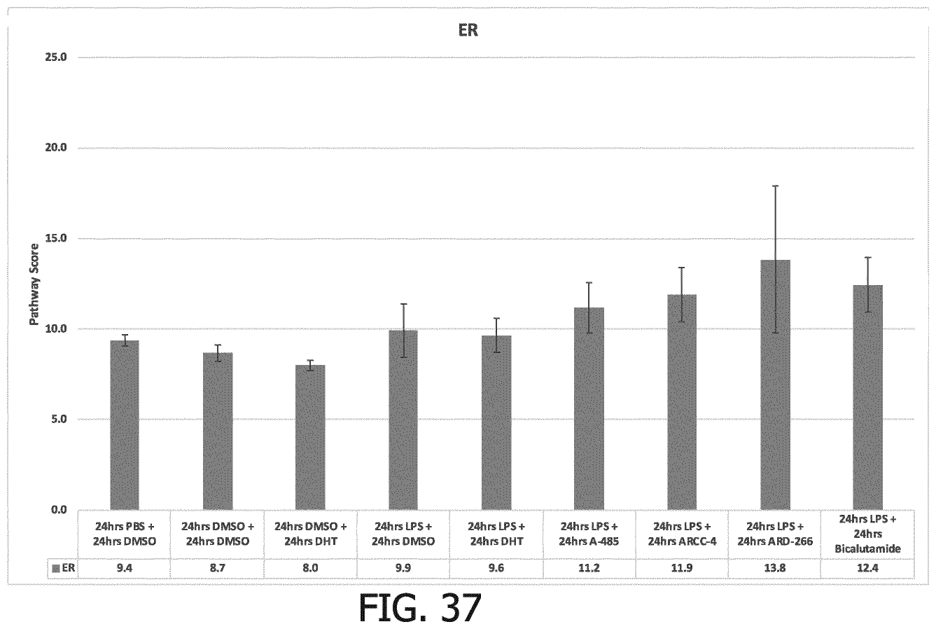

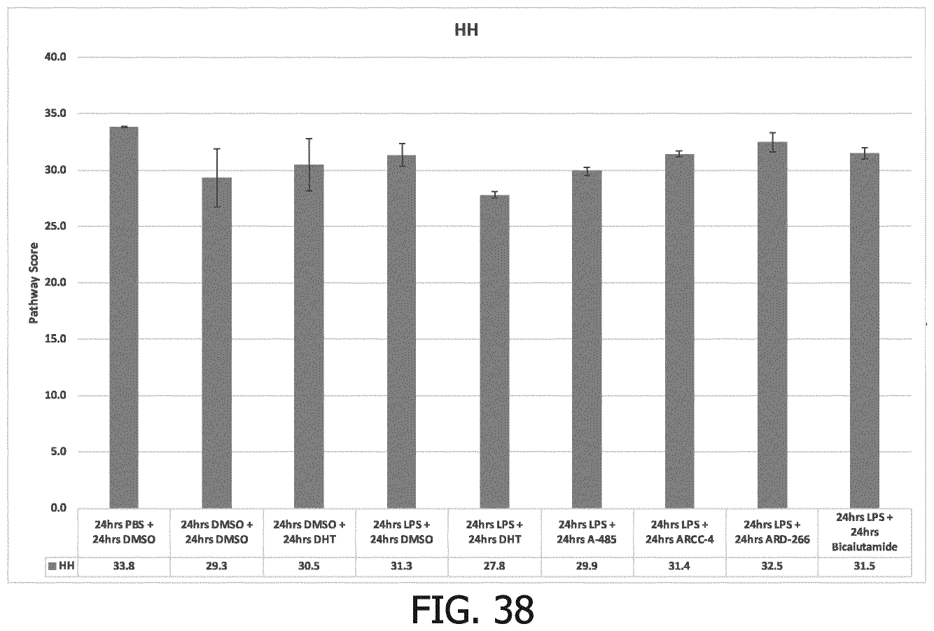

FIGS. 37 and 38 demonstrate that the ER and HH cellular signaling pathway activities are not substantially affected by either LPS, DHT or the AR pathway inhibitors, therefore demonstrating that the effect shown in FIG. 36 is specific.

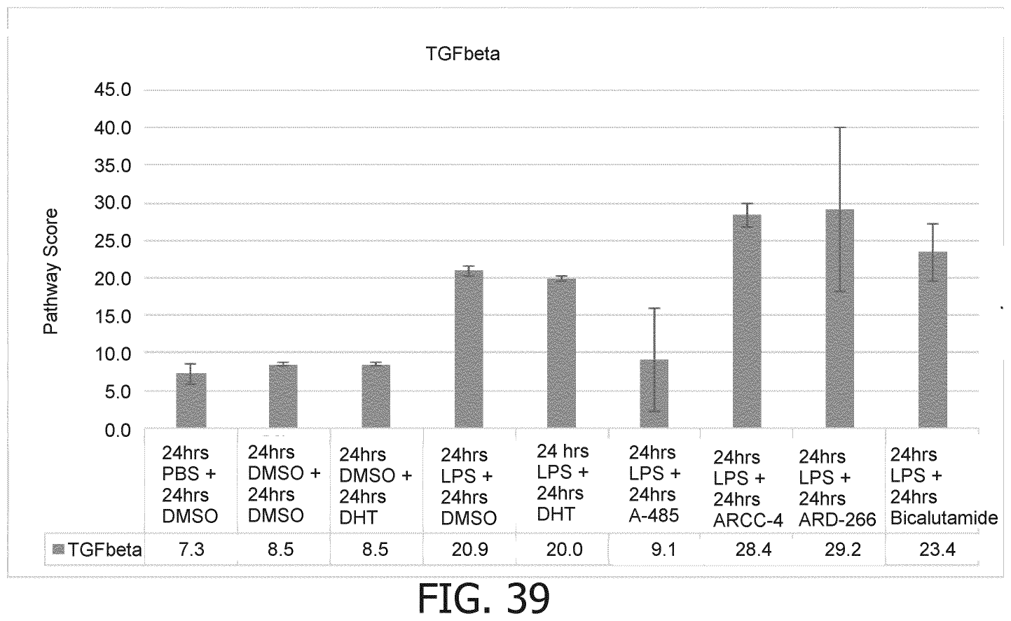

FIG. 39 shows that also TGFbeta signaling is increased by LPS, which is in line with other data shown herein wherein it is demonstrated that sepsis affects both AR and TGFbeta pathways. As expected, DHT did not increase TGFbeta activity. Interestingly, A-458 shows a reduction of LPS induced TGFbeta activity as well as a reduction of AR activity, suggesting it functions as a dual AR/TGFbeta inhibitor. As expected the remaining AR inhibitors were not able to mitigate the effect of LPS on TGFbeta cellular signaling pathway activity.

From these data it can be concluded that sepsis elevates AR and TGFbeta cellular signaling pathway activities in blood cells, which can detect in a patient's blood sample and used for quick diagnosis of sepsis or prediction of patients at risk of developing sepsis. Further, these data demonstrate that the elevated AR and TGFbeta can at least partially be attributed to monocytes, and that the effect can be recreated by adding LPS to cultured monocytes. Further these data demonstrate that LPS induced increased AR signaling pathway activity can be mitigated by an AR pathway inhibitor, as demonstrated by in vitro experiments using monocytes. This demonstrates that AR inhibitors can likely be used to treat, or at least reduce the symptoms (alleviate), of a subject with sepsis, on the premise that the patient has an increased AR pathway activity or abnormal expression of the sepsis-associated genes. It further emphasizes the need for a companion test to identify patients at risk of developing sepsis or patients with sepsis who would benefit from treatment with an AR inhibitor.

### Example 11—Screening of Pathway Inhibitors for Preventing or Treating Sepsis in a THP-1 Model for Sepsis

### Introduction

Sepsis is a life-threatening infection in which the immune response is dysregulated resulting in multi-organ dysfunction or failure [17]. Sepsis is generally a complication of severe bacterial infection and characterized by a systemic inflammatory response leading to septic shock. Mortality rates range between 25 and 30% for sepsis and 40% to 70% for septic shock [4], [18], [19].

Aside from antibiotics and supportive measures to maintain blood circulation of internal organs, no treatments have proven to be effective, although it cannot be excluded that some treatments may benefit a subset of patients who so far cannot be identified [17]. One reason for failure to develop effective treatments is the heterogeneity among sepsis patients, that is, variation in underlying medical conditions and use of drugs, and genetic variations influencing the immune response in an individual patient.

Detailed assessment of the functional immune response in a patient with sepsis may enable a personalized treatment approach and improve treatment efficacy. Diagnostic assessment of immune function is currently limited to routine blood measurements, such as numbers of immune cells and inflammation markers (e.g., C-reactive protein), but is not informative on the functional activity state of the various types of immune cells, responsible for the abnormal immune response in a sepsis patient.

The functional state of immune cells is determined by a small number of so-called cellular signal transduction pathways (STPs) [20], [21], [22], [23], [24]. Recently, novel assays have been developed to quantitatively measure activity of STPs in cell and tissue samples, including blood samples [25], [26], [27], [28]. Measuring combined activity of these STPs in blood cells is expected to enable quantitative assessment of the innate and adaptive immune response in an individual patient [23], [29].

STP analysis was performed on publicly available gene expression data from multiple clinical sepsis studies [30]. Studies shows that activity of the AR and TGFbeta was increased in sepsis patients compared to healthy controls, indicating that these pathways represent novel drug targets to treat or prevent sepsis.

Treatment with a pathway inhibitor, particular an AR and TGFbeta pathway inhibitor, may be beneficial for subjects having sepsis or in danger of developing sepsis. Treatment of patients with sepsis with AR inhibitors has been tried before but was unsuccessful. Based on our results this can be explained by the fact that not all sepsis patients exhibit high AR pathway activity, and therefore only those with strongly increased AR pathway activity might benefit from treatment with an AR pathway inhibitor. More importantly, patients with an infection who are at risk of developing sepsis and have high AR pathway activity in relevant blood cells may benefit from preventive treatment with an AR inhibitor. The rationale behind this assumption is that an active AR pathway results in immunosuppression, see e.g. Gubbels Bupp and Jorgensen [5], incorporated by reference in its entirety.

It was found that AR proteins are expressed in a wide variety of innate and adaptive immune cells including neutrophils, macrophages, mast cells, monocytes, megakaryocytes, B cells, and T cells, suggesting that the AR pathway may indeed be ligand-inducible. Interestingly, AR proteins are expressed also in hematopoietic stem cells and lymphoid and myeloid progenitor cells. Evidence derived from different studies points to an immunosuppressive role of androgens in different immune cell types mostly by changing expression of pro-inflammatory and anti-inflammatory mediators which are important for an appropriate immune response.

Concurrent with the pro-inflammatory response in sepsis, there is evidence of a number of immune suppressive events occurring in which immune suppressive cytokines such as IL-10, TGF-β, and IL6 play a role. According to a clinical observational cohort study, septic patients can develop chronic critical illness, with a 6-month survival of 63%, and continue to demonstrate cytokine profiles of chronic inflammation, as well as biomarker profiles characteristic for persistent immunosuppression [31]. In the study from Hiraki et al. [32], a murine abdominal sepsis model was utilized in which a TGF-β depleting antibody was administered leading to improved survival of the mice. This suggests a causal role for the TGFbeta pathway in sepsis. In summary, AR and TGFbeta pathways may play a causal role in sepsis by modifying the immune response towards immune suppression and a pro-inflammatory state.

In view of the clinical heterogeneity between sepsis patients, as already shown before, personalized treatment is likely to be necessary to obtain clinical benefit. Until now it was not possible to stratify patients that would benefit from AR or TGFbeta pathway inhibitors for prevention or treatment of sepsis, however the methods described herein allow for an accurate assessment of the AR and TGFbeta pathway activity in a blood sample. This allows discrimination between patients with low and high AR and or TGFbeta pathway activity and administering inhibitors which will inhibit the AR and or TGFbeta to those with high AR and or TGFbeta pathway activity in order to normalize pathway activity.

### Drug Repurposing Screen for Sepsis

In order to develop novel rational therapies to prevent and treat sepsis in a personalized manner, several laboratory models for aberrant immune cell (monocyte) function in sepsis were investigated:
A monocytic cell line (THP-1), stimulated with lipopolysaccharide (LPS)
Primary blood-derived monocytes, stimulated with LPS
Healthy volunteer-derived PBMC's, stimulated with LPS
Sepsis patient-derived PBMC's

The effect of LPS on STP activity in the laboratory sepsis models was compared with the previously described STP activity in blood cells (whole blood samples) of patients with sepsis. This analysis confirmed that LPS in a dose-dependent manner activates AR and TGFbeta STPs in the THP-1 and primary monocytic cell models for sepsis (Tables 7-10). AR and TGFbeta pathway activity were only induced in monocytic cells by LPS and not in the lymphoid cell line (MOLT-4, see Table 10). As predicted, LPS did not have an effect on activity of these STPs in PBMCs, due to monocytes making up only 5-10% of the cells in PBMC samples, other cells being lymphocytes (70-90%), resulting in too much dilution of the effect on monocytes [33]. The difference in results between monocytic cells and PBMCs also emphasizes the specificity of the measured differences in STP activity.

Addition of original ligands for the AR and TGFbeta STPs to the monocytic cell model also confirmed that the AR and TGFbeta pathways can indeed be properly activated in monocytic cells, and are therefore functional STPs in this cell type. Based on these results we confirm the AR and TGFbeta STPs as potential drug targets for sepsis, in monocytic cells.

Subsequently we proceeded to screen drugs which could stand-alone or in combination correct abnormal AR and TGFbeta STP activity in these laboratory sepsis models. We investigated their potential use for two clinical applications:
1. Prevention of sepsis;
2. Treatment of established sepsis.

For the first application, THP-1 cells were pre-incubated for 24 hours with the drug of choice, and subsequently LPS was added for 24 hours. For the treatment application, the drug and LPS were added simultaneously to the cells for 24 hours. Importantly: Some drugs needs processing in the body to become effective, this is the case for some of the androgen receptor (AR) pathway inhibitors, such as bicalutamide. In this case we have also tested the active variant of the drug (in the case of bicalutamide: (R-)bicalutamide).

The following drugs (or drug combinations) were tested in the THP-1 monocytic sepsis model by reducing LPS-induced activity of the AR and/or TGFbeta STPs: In the sepsis prevention model: Bicalutimide and vactosertib were tested (Table 10). In the sepsis treatment model: (R)-Bicalutamide, Ketodarolutamide (ORM-15341), D4-abiraterone, Etanercept, Resatorvid, Vactosertib, N-desmethyl Enzalutamide, N-Desmethyl-Apalutamide, R-bicalutamide+Etanercept and R-bicalutamide+Filgotinib (Table 11).

### Effects of Drugs on PBMC Samples of Sepsis Patients

The following drugs (or drug combinations) were tested in PBMC and showed reduction in the AR and or TGFbeta STPs. For the TGFbeta pathway the following compounds showed significant decrease: Vactosertib (p=0.0079), Vactosertib+R-bicalutamide (p=0.0004), D4-abiraterone (p=0.0527), Galunisertib (p=0.0013), D4-abiraterone+Vactosertib (p=0.0040), Vactosertib+Etanercept (10 ug/ml) (p=0.0270) and D4-abiraterone+Galunisertib (p=0.0075) using paired single sided t-test. (Table 13) Both AR and TGFbeta pathway was significant lower with Galunisertib (AR p=0.0302). The following compounds showed reduction in the AR pathway but were not significant; Vactosertib 2/3 patients, Vactosertib+R-bicalutamide in 3/3 patients and Vactosertib+Etanercept (10 ug/ml) 4/5 patients. (Table 13)

In sepsis patients also other factors besides LPS play a role in the symptomatology, like a reduced immune suppressive status, reflected in reduced function of the adaptive immune system (lymphocytes) and increased activity of the innate, inflammatory, immune (monocytic lineage) system.

It is known that TGFbeta induces an inflammatory state of monocytes and an immune suppressed state of lymphocytes[41], [23]). Using the here described drugs (and combinations), we can reduce the inflammatory phenotype of monocytes and the immune suppressive phenotype of lymphocytes. Envisioned use of these drugs to prevent and treat sepsis: important to consider is that in a severely ill sepsis patient it may not be possible or preferable to orally administer a drug, in view of inability for oral administration of drugs and inadequate gastro-intestinal drug absorption. In a patient at risk of sepsis oral administration is possible. Thus, orally effective drugs can potentially be used to prevent sepsis in infected patients. For treatment of a patient with sepsis or septic shock the formulation may need to be changed to an intravenous drug preferably, or alternatively maybe subcutaneous or intramuscular formulation.

Thus, while these drugs are all available for oral administration and may be repurposed for prevention of sepsis and treatment of early sepsis, development of another formulation (intravenous, subcutaneous, intramuscular) is preferred for the treatment of severely ill sepsis patients. The most effective dose, both for oral administration as for intravenous/subcutaneous/intramuscular can be determined in in regular dose effect studies.

### Methods

### THP-1 Sepsis Model

LPS is considered the main mediator of the sepsis symptoms. An in vitro experiment was designed where monocytes were stimulated with LPS and subsequently treated with a variety of inhibitors. Monocytes were chosen as they have been described to play a major role in sepsis, see for example Sukhacheva, [34], Clinical Laboratory Int. 26 Aug. 2020 or Haverman et al.[15], The central role of monocytes in the pathogenesis of sepsis: consequences for immunomonitoring and treatment, The Netherlands Journal of Medicine, Volume 55, Issue 3, September 1999, Pages 132-141. To study the inflammation process we stimulated the monocytic THP-1 cells (ATCC® TIB-202™) using DMSO or LPS (0.5 or 5 ng/ml, originated from E. coli) or LPS in combination with drugs into culture medium (DMEM, supplemented with 10% FBS, 1% glutamax and 1% pen strep at 37° C. 5% CO2.).

Sepsis prevention model: THP-1 cells were pre-incubated for 24 hours with the drug of choice or DMSO and subsequently cultured for 24 hours with LPS or PBS. Compounds and concentrations are described in Table 9.

Sepsis treatment model: For the treatment application, the drug and LPS were added simultaneously to the cells for 24 hours. Compounds and concentrations are described in Table 11 and Table 12 for healthy volunteer PBMCs.

### MOLT-4 Cell Line:

MOLT-4 (ATCC CRL-1582) were cultured in RPMI1640 media, supplemented with 10% FBS, 1% glutamax and 1% pen strep at 37° C. 5% CO2.

### RNA Extraction and STP Activity Scores

After stimulation cells were harvested and RNA was extracted using the RNeasy mini kit (Qiagen, 74104). qPCR-based STP activity scores were calculated using the Philips Research OncoSignal platform (www.philips.com/oncosignal), as described herein.

Measurement of Activity of Signal Transduction Pathways on Affymetrix Microarray Data from Cell Samples.

Pathway activity scores (PAS) were calculated from Affymetrix expression microarray data derived from the GEO database (https://www.ncbi.nlm.nih.gov/gds/) [35]. For each signaling pathway, normalized PAS were presented on a log 2 odds scale. The log 2 odds score for pathway activity is derived from the probability score for activity of the pathway-associated transcription factor calculated by the computational model, as described [36], [37], [38].

### Microarray Data Quality Control

Quality control (QC) was performed on Affymetrix data of each individual sample, as described before [38]. In summary, QC parameters include: the average value of all probe intensities, presence of negative, or extremely high (>16-bit) intensity values, poly-A RNA (sample preparation spike-ins), and labelled cRNA (hybridization spike ins) controls, GAPDH, and ACTB 3′/5′ ratio, centre of intensity and values of positive and negative border controls determined by affyQCReport package in R, and an RNA degradation value determined by the AffyRNAdeg function from the Affymetrix package in R [39], [40]. Sample data that failed QC were removed prior to data analysis.

PBMCs Derived from Healthy and Sepsis Patients

PBMC samples from healthy volunteers and sepsis patients were obtained via Tissue solutions. Briefly, cryopreserved PBMCs were first washed with washing buffer (PBS/1% BSA/2 mM EDTA), centrifuged and pellet resuspended in culture medium (RPM11640 without phenol red+8.5% charcoal stripped FBS+1% Glx+1% p/s). 0.5·10\hat{ }5-1·10\hat{ }6 cells were seeded per well in 24 wells plate and rested for 1 hour before treatment experiment. 10 PBMC samples from healthy donors were used to; (1) measure the baseline pathway levels, (2) activate with LPS

Sepsis treatment model: PBMC derived from sepsis patients (n=20) were treated 24 hours with compounds, DMSO or combination of compounds. (PBMC from healthy and sepsis patients are originating from the same hospital to minimize bias in sample prep). After stimulation cells were harvested and RNA was extracted using the RNeasy mini kit (Qiagen, 74104). qPCR was performed using the Philips Research OncoSignal platform.

### Results

### Functional AR and TGFbeta STPs in Monocytic Cells

AR and TGFbeta pathways are functional in monocytic cells (Table 7). Testosterone induced activity of the AR pathway in THP-1 cells, which could be inhibited by bicalutamide. TGFbeta induced activity of the TGFbeta pathway activity, which was inhibited by a specific and known TGFbeta pathway inhibitor. In conclusion, both pathways were shown to be functional in monocytic cells.

TABLE 7
AR (A) and TGFBeta (B) STPs are functional in THP-1 cells.
STP scores in normalized scores. THP-1 cell stimulated for
24 hours with DMSO or LPS and subsequently with DMSO or DHT.

| | A | B |
|---|---|---|
| Group | AR | TGFbeta |
| 24 hrs DMSO + 24 hrs DMSO | 1.4 | 8.3 |
| 24 hrs DMSO + 24 hrs DMSO | 1.2 | 8.6 |
| 24 hrs DMSO + 24 hrs DHT (25 nM) | 9.9 | 8.5 |
| 24 hrs DMSO + 24 hrs DHT (25 nM) | 9.8 | 8.5 |
| 24 hrs LPS (50 ng/ml) + 24 hrs DMSO | 18.1 | 20.4 |
| 24 hrs LPS (50 ng/ml) + 24 hrs DMSO | 19.4 | 21.4 |
| 24 hrs LPS (50 ng/ml) + 24 hrs DHT (25 nM) | 21.2 | 20.2 |
| 24 hrs LPS (50 ng/ml) + 24 hrs DHT (25 nM) | 21.6 | 19.8 |

### Monocytic/Monocyte-Based Sepsis Models

### LPS Increases AR and TGFbeta STP Activity in Monocytic/Monocyte Sepsis Models

In THP-1 monocytic cells the STP activity was increased in a dose-dependent manner for the pathways AR and TGFBeta in the LPS stimulated cells using 5 and 0.5 ng/ml LPS (Table 8A). Increase of AR, TGFbeta STP activity by LPS was similarly found in primary monocytes derived from human volunteers, stimulated 6 hours with 10 ng/ml LPS (GEO database dataset GSE84161) (Table 8B). AR and TGFbeta pathway activity were only induced in monocytic cells by LPS and not in lymphoid cells (MOLT-4). (Table: 9)

TABLE 8
LPS increases AR and TGFbeta STP activity scores in a dose-dependent manner.
A) THP-1 cells. DMSO, 5 and 0.5 ng/ml LPS stimulation. STP scores in normalized
scores. B) Primary monocytes untreated and treated with LPS (10 ng/ml)
for 6 hours (GEO dataset GSE84161). STP scores in log2odds
A

| | Average | | STDEV | |
|---|---|---|---|---|
| Group | AR | TGFbeta | AR | TGFbeta |
| 24 hrs DMSO + 24 hrs PBS | 5.0 | 2.3 | 0.3 | 0.5 |
| 24 hrs DMSO + 24 hrs 0.5 ng/ml LPS | 9.6 | 6.8 | 1.1 | 2.5 |
| 24 hrs DMSO + 24 hrs 5 ng/ml LPS | 16.7 | 14.2 | 0.7 | 1.4 |

B

| Group | AR | TGFbeta |
|---|---|---|
| LPS 1 | −10.80 | 6.66 |
| LPS 2 | −10.03 | 9.79 |
| LPS 3 | −9.56 | 10.25 |
| LPS 4 | −10.38 | 7.46 |
| LPS 5 | −9.14 | 4.76 |
| Untreated 1 | −12.44 | −1.91 |
| Untreated 2 | −12.29 | −3.52 |
| Untreated 3 | −10.69 | −0.81 |
| Untreated 4 | −11.11 | −3.81 |
| Untreated 5 | −11.86 | −3.69 |

TABLE 9
AR and TGFbeta pathway activity is only induced in monocytic
cells (THP-1) by LPS and not lymphoid cells (MOLT-4).
THP 1 (A) and MOLT-4 (B) cell line stimulated with LPS,
DHT or DMSO for 5 hours and subsequently inhibited with
Bicalutamide or Enzalutamide. Normalized. AR and TGFBeta
STP activity shown in normalized scores.

| | | |
|---|---|---|
| Group | AR | TGFbeta |

A

| | | | |
|---|---|---|---|
| THP-1 | unstimulated (DMSO) | 0.3 | 15.5 |
| | unstimulated (DMSO) | 0.3 | 16.7 |
| | LPS (50 ng/mL) | 22.3 | 52.4 |
| | LPS (50 ng/mL) + Bicalutamide (5 uM) | 20.2 | 34.6 |
| | LPS (50 ng/mL) + Enzalutamide (5 uM) | 20.4 | 33.6 |
| | DHT (25 nM) | 8.7 | 12.9 |
| | DHT (25 nM) + Bicalutamide (5 uM) | 6.7 | 15.6 |
| | DHT (25 nM) + Enzalutamide (5 uM) | 6.0 | 17.3 |

B

| | | | |
|---|---|---|---|
| Molt-4 | unstimulated (DMSO) | 7.2 | 14.3 |
| | unstimulated (DMSO) | 7.4 | 14.1 |
| | LPS (50 ng/mL) | 6.9 | 14.0 |
| | LPS (50 ng/mL) + Bicalutamide (5 uM) | 7.2 | 14.1 |
| | LPS (50 ng/mL) + Enzalutamide (5 uM) | 6.3 | 14.1 |
| | DHT (25 nM) | 7.5 | 14.1 |
| | DHT (25 nM) + Bicalutamide (5 uM) | 7.4 | 13.9 |
| | DHT (25 nM) + Enzalutamide (5 uM) | 7.3 | 14.1 |

### Drug Discovery:

Drugs which Inhibit LPS-Induced AR and TGFbeta STPs in the THP-1 Sepsis Models (Prevention and Treatment)

Based on the above described data it was hypothesized that sepsis may be prevented and treated by administering drug(s) which inhibit AR and/or TGFbeta cellular signaling pathway activity.

### Prevention of Sepsis

Monocyte cells were first cultured for 24 hours with 25 uM Bicalutamide or DMSO and subsequently cultured with LPS, DHT or PBS. the experiment was performed in triplicate and repeated for two times to obtain robust results.

As shown in Table 10, pretreatment of bicalutamide alone did not result in increasement of any of the pathways compared to DMSO. Both LPS and DHT increased AR cellular signaling pathway activity in monocytes, LPS also activates TGFbeta. Pretreatment of bicalutamide followed by LPS or DHT stimulation resulted in significant lower activation of AR pathway activity by LPS (bicalutamide AR; p=0.04, DHT AR; p=0.0003, n=9) compared with LPS without pre-treatment. Interestingly, bicalutamide shows a reduction of LPS induced TGFbeta activity as well as a reduction of AR activity, suggesting it functions as a dual AR/TGFbeta inhibitor.

TABLE 10
Effect of drugs on THP-1 sepsis prevention model. A. Bicalutamide;
B. Vactosertib. STP scores in normalized scores.
A

| | Average | | STDEV | |
|---|---|---|---|---|
| Group: | AR | TGFbeta | AR | TGFbeta |
| 24 hrs Bical + 24 hrs DHT | 3.17 | 1.04 | 1.62 | 0.48 |
| 24 hrs Bical + 24 hrs LPS | 12.93 | 9.78 | 1.97 | 2.54 |
| 24 hrs Bical + 24 hrs PBS | 2.63 | 1.25 | 1.08 | 0.48 |
| 24 hrs DMSO + 24 hrs DHT | 6.13 | 1.27 | 1.64 | 0.59 |
| 24 hrs DMSO + 24 hrs LPS | 14.50 | 11.83 | 1.64 | 3.53 |
| 24 hrs DMSO + 24 hrs PBS | 1.62 | 1.16 | 0.46 | 0.42 |

B

| | Average PAS | | STDEV PAS | |
|---|---|---|---|---|
| Group: | AR | TGFbeta | AR | TGFbeta |
| 24 hrs DMSO + 24 hrs PBS | 4.5 | 2.6 | 1.1 | 1.2 |
| 24 hrs DMSO + 24 hrs LPS | 17.0 | 13.5 | 1.0 | 0.3 |
| 24 hrs Vactosertib + 24 hrs PBS | 3.8 | 1.7 | 0.3 | 0.3 |
| 24 hrs Vactosertib + 24 hrs LPS | 17.1 | 9.3 | 0.9 | 0.7 |

### TGFbeta Inhibitors for Prevention of Sepsis

Using the similar approach as for AR inhibitors (first 24 inhibitor followed by 24 h LPS stimulation),

As shown in Table 10B pretreatment of Vactosertib alone did not result in increasement of any of the pathways compared to DMSO. LPS increased both AR and TGFbeta. Pretreatment of Vactosertib followed by LPS stimulation resulted in significant lower activation of TGFbeta pathway activity (p=1.3E-05 single sided t-test) compared without pre-treatment. No effect of Vactosertib was seen on the AR signaling pathway.

The results demonstrate that LPS induced increased AR and TGFbeta signaling pathway activity can be mitigated by an AR pathway inhibitor, as demonstrated by in vitro experiments using monocytes. TGFbeta signaling pathway activity can be mitigated using an TGFbeta pathway inhibitor. This demonstrates that drugs/compounds which inhibit AR and TGFbeta STPs can likely be used to prevent sepsis in a patient who is at high risk to develop sepsis.

### Treatment of Sepsis

Two concentrations of LPS 0.5 and 5 ng/ml were used in the experiments, furthermore for the THP-1 cell line an higher passage number (px-53-55) and a lower passage number (px-13-15) were used (Table 11). the THP-1 cell line with the lower passage was more reactive towards LPS activation compared to the higher passage number (data not shown).

### THP-1 Monocyte Sepsis Model:

For the AR signaling pathway the following compounds could reduce the AR PAS; (R)-Bicalutamide, Ketodarolutamide, D4-abiraterone, Etanercept, Resatorvid, Vactosertib, N-Desmethyl-Apalutamide and N-desmethyl Enzalutamide, separate and the combinations (R)-bicalutamide+Etanercept and (R)-bicalutamide+Filgotinib could lower the pathway activity scores in the THP-1 monocytes. (Table 11).

For the TGFbeta signaling pathway; (R)-Bicalutamide, D4-abiraterone, Etanercept, Filgotinib, Resatorvid and vactosertib could lower the PAS. (Table 11).

Both AR and TGFbeta pathway could be lowered by; Etanercept, Resatorvid and (R)-bicalutamide+Etanercept (Table 11).

TABLE 11
THP-1 cells stimulated with DMSO (vehicle control), LPS, or LPPS in
combination with compounds. AR and TGFBeta STP activity scores and
STDEV of STP scores are shown. Each block represents an experiment;
blocks are separated by an empty row. STP scores in normalized scores.

| | | | | | LPS | |
|---|---|---|---|---|---|---|
| | Average | | STDEV | | conc. | THP-1 |
| Compound | AR | TGFb | AR | TGFb | (ng/ml) | cell line |
| DMSO-PBS (placebo control) | 3.00 | 11.67 | 0.00 | 0.58 | 0.5 | px 53-55 |
| LPS (sepsis) | 4.17 | 12.17 | 1.94 | 0.75 | 0.5 | px 53-55 |
| (R)-Bicalutamide (25 uM) + LPS | 1.67 | 11.67 | 1.15 | 0.58 | 0.5 | px 53-55 |
| Ketodarolutamide (10 uM) + LPS | 2.33 | 12.00 | 0.58 | 0.00 | 0.5 | px 53-55 |
| D4-abiraterone (10 uM) + LPS | 2.67 | 11.67 | 0.58 | 0.58 | 0.5 | px 53-55 |
| DMSO-PBS (placebo control) | 3.67 | 7.67 | 0.58 | 0.58 | 0.5 | px 13-15 |
| LPS (sepsis) | 10.50 | 13.17 | 1.87 | 0.98 | 0.5 | px 13-15 |
| Etanercept (10 ug/ml) + LPS | 6.33 | 11.33 | 0.58 | 0.58 | 0.5 | px 13-15 |
| DMSO-PBS (placebo control) | 2.67 | 8.67 | 0.58 | 0.58 | 5 | px 13-15 |
| LPS (sepsis) | 14.00 | 15.67 | 0.00 | 1.53 | 5 | px 13-15 |
| Resatorvid (10 uM) + LPS | 3.67 | 8.00 | 0.58 | 1.73 | 5 | px 13-15 |
| PBS (placebo control) | 1.33 | 7.67 | 0.58 | 1.53 | 0.5 | px 13-15 |
| LPS (sepsis) | 10.33 | 13.67 | 0.58 | 0.58 | 0.5 | px 13-15 |
| Vactosertib (1 uM) + LPS | 8.67 | 12.00 | 2.08 | 1.00 | 0.5 | px 13-15 |
| Etanercept (1 ug/ml) + LPS | 5.00 | 9.67 | 1.00 | 0.58 | 0.5 | px 13-15 |
| Etanercept (1 ug/ml) + LPS | 6.00 | 11.67 | 1.00 | 0.58 | 0.5 | px 13-15 |
| R-bicalutamide (25 uM) + | 4.33 | 11.00 | 1.53 | 1.73 | 0.5 | px 13-15 |
| Etanercept (0.1 ug/ml) + LPS | | | | | | |
| R-bicalutamide (25 uM) + | 7.33 | 14.00 | 1.15 | 0.00 | 0.5 | px 13-15 |
| Filgotinib (10 uM) + LPS | | | | | | |
| DMSO-PBS (placebo control) | 4.67 | 13.00 | 0.58 | 0.00 | 0.5 | px 13-15 |
| LPS | 9.00 | 13.00 | 2.89 | 1.00 | 0.5 | px 13-15 |
| (R)-Bicalutamide (25 uM) + LPS | 6.67 | 14.00 | 0.58 | 0.00 | 0.5 | px 13-15 |
| N-desmethyl Enzalutamide | 6.67 | 13.33 | 2.31 | 1.15 | 0.5 | px 13-15 |
| (2 uM) + LPS | | | | | | |
| N-Desmethyl-Apalutamide | 7.33 | 13.33 | 1.53 | 0.58 | 0.5 | px 13-15 |
| (3 uM) + LPS | | | | | | |

### PBMCs Healthy Volunteers:

PBMCs of healthy volunteers were stimulated with 5 ng/ml LPS for 24 hours in combination with (R)-Bicalutamide and (R)-Bicalutamide+Vactosertib (experiment 1). In experiment 2, (R)-Bicalutamide and etanercept were tested with 0.5 ng/ml LPS (Table 12A for exp 1 and Table 12B for exp 2). 0.5 and 5 ng/ml LPS did not significantly increase the AR and TGFbeta PAS, which was shown in the THP-1 sepsis model.

Therefore the LPS-stimulated PBMC model was considered to not be a good model for sepsis. For completeness sake we have added results on treatment with drugs.

In PBMC samples, monocytes represent only 5-10% of cells, most cells in PBMC represents the T-cells with 40-60% o. Probably the monocytes are too much diluted in the PBMC sample to see a clear overall effect of LPS increased PAS. The effects of the compounds below could be a representation of the monocytes or the combination effect of also the T-cells.

(R)-Bicalutamide+Vactosertib showed significant lower (paired t-test) AR (p=1E-05) and TGFbeta (p=5E-04) PAS compared to LPS stimulated PBMCs. (R)-Bicalutamide showed an AR and TGFbeta decrease in 3 out of 6 Healthy volunteers in experiment 1 and in 3 out of 4 in experiment 2 (exp2: bicalutamide+LPS: AR p=5E-02). Etanercept lowered (not significantly) AR PAS in 2 out of 4 and TGFbeta PAS in 3 out of 4 Healthy volunteers.

TABLE 12
A). PAS of PBMCs from healthy volunteers stimulated with DMSO, 5
ng/ml LPS for 24 hours or in combination with (R)-Bicalutamide or
(R)-Bicalutamide + Vactosertib. B). Etanercept or Bicalutamide.
A

| | | | | | LPS + (R)- |
|---|---|---|---|---|---|
| | | | | | Bicalutamide |
| | | | | LPS + (R)- | (25 uM) + |
| | Healthy | DMSO | LPS | Bicalutamide | Vactosertib |
| | volunteers IDs | (0.1%) | (5 ng/ml) | (25 uM) | (1 uM) |
| AR | CIDp780001566 (M) | 30 | 31 | 29 | 22 |
| | CIDp780001567 (F) | 37 | 27 | 26 | 20 |
| | CIDp780001568 (F) | 28 | 28 | 30 | 21 |
| | CIDp780001649 (F) | 22 | 25 | 25 | 19 |
| | CIDp780001695 (M) | 26 | 28 | 26 | 22 |
| | CIDp780005323 (F) | 25 | 26 | 21 | 20 |
| TGFbeta | CIDp780001566 (M) | 30 | 30 | 29 | 15 |
| | CIDp780001567 (F) | 36 | 34 | 32 | 18 |
| | CIDp780001568 (F) | 41 | 39 | 35 | 24 |
| | CIDp780001649 (F) | 31 | 32 | 31 | 16 |
| | CIDp780001695 (M) | 30 | 27 | 27 | 15 |
| | CIDp780005323 (F) | 29 | 33 | 40 | 29 |

B

| | | | | LPS + | LPS + (R)- |
|---|---|---|---|---|---|
| | Healthy | DMSO | LPS | Etanercept | Bicalutamide |
| | volunteers IDs | (0.1%) | (0.5 ng/ml) | (10 ug/ml) | (25 uM) |
| AR | CIDp780005324 (F) | 26 | 29 | 21 | 26 |
| | CIDp780005325 (M) | 28 | 28 | 31 | 28 |
| | CIDp780005327 (M) | 31 | 31 | 31 | 30 |
| | CIDp780005331 (M) | 20 | 26 | 18 | 24 |
| TGFbeta | CIDp780005324 (F) | 32 | 32 | 29 | 31 |
| | CIDp780005325 (M) | 37 | 31 | 34 | 35 |
| | CIDp780005327 (M) | 33 | 33 | 31 | 33 |
| | CIDp780005331 (M) | 31 | 34 | 31 | 32 |

### PBMCs Sepsis Patients:

To investigate the effect of selected drugs which were effective in the THP-1 sepsis model to counteract the stimulatory effect of LPS on AR and TGFbeta pathway activity, subsequently PBMCs of actual sepsis patients were treated in vitro with these drugs and drug combinations.

Although LPS-stimulated PBMCs of healthy volunteers were considered to be not a good laboratory model system for sepsis, we decided to investigate the effect on PBMCs from actual patients with sepsis, for the following reasons. LPS is not the only abnormal factor present in the blood of sepsis patients; for example other bacterial-derived molecules such as Peptidoglycan (PepG) and lipoteichoic acid (LTA) or LPS-induced secondary molecules, are likely to affect STP activity in the lymphocytic immune cells in addition to monocytic cells. Also we hypothesized before that at least a subset of patients with a bacterial infection who develop sepsis are likely to have an immune-suppressed immune system[41], [23]. At least some lymphocytes exhibit increased TGFbeta pathway activity and potentially AR pathway activity upon immune-suppression [23], [24].

In total 18 PBMCs sepsis samples were treated for 24 hours with selected inhibitors (and combinations), 6 patient samples per experiment.

Experiment 1 (Table 13: A, B): Vactosertib, Vactosertib+R-bicalutamide, Etanercept (10 ug/ml) and R-bicalutamide.

Experiment 2 (Table 13: C, D): Resatorvid, D4-abiraterone, A-485 and Galunisertib.

Experiment 3 (Table 13: E, F): D4-abiraterone+Vactosertib, R-bicalutamide+Etanercept (10 ug/ml), Vactosertib+Etanercept (10 ug/ml) and D4-abiraterone+Galunisertib.

Unfortunate due to the poor sample quality of the sepsis patients the pathway activity could not be measured for all patients. This also limited the statistics with the low sample size per experiment.

For the TGFbeta pathway the following compounds showed significant decrease in pathway activity: Vactosertib (p=0.0079), Vactosertib+R-bicalutamide (p=0.0004), D4-abiraterone (p=0.0527), Galunisertib (p=0.0013), D4-abiraterone+Vactosertib (p=0.0040), Vactosertib+Etanercept (10 ug/ml) (p=0.0270) and D4-abiraterone+Galunisertib (p=0.0075).

Pathway activity scores of both AR and TGFbeta pathways were significant lower with Galunisertib (AR p=0.0302).

The following compounds showed reduction in the AR pathway but the reduction was not significant; Vactosertib 2/3 patients, Vactosertib+R-bicalutamide in 3/3 patients and Vactosertib+Etanercept (10 ug/ml) 4/5 patients.

As mentioned above, in sepsis patients also other factors besides LPS play a role in the symptomatology, like a reduced immune suppressive status, reflected in reduced function of the adaptive immune system (lymphocytes) and increased activity of the innate, inflammatory, immune (monocytic lineage) system.

It is known that TGFbeta induces an inflammatory state of monocytes and an immune suppressed state of lymphocytes [23]. Using the here described drugs (and combinations), we can reduce the inflammatory phenotype of monocytes and the immune suppressive phenotype of lymphocytes. Since we believe that an immune suppressive state predisposes to development of and worse clinical outcome of sepsis, the effective drugs and drug combinations are likely to be effective both to prevent development of sepsis inpatients with a bacterial infection, and to improve clinical outcome of sepsis, by restoring the functionality of the adaptive immune response and the innate immune response [4]. By prevention of this status both sepsis as long term immune suppressive effects can be prevented.

| | A | | | | |
|---|---|---|---|---|---|
| | AR Pathway activity scores of sepsis PBMC patients | | | | |
| | | | Vactosertib (1 uM) + | | R- |
| EXP 1 | | Vactosertib | R-bicalutamide | Etanercept | bicalutamide |
| AR | DMSO | (1 uM) | (25 uM) | (10 ug/ml) | (25 uM) |
| CIDp780000198 | 28 | 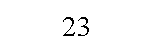 | 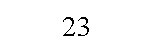 | 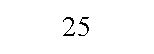 | 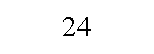 |
| CIDp780000197 | | | 29 | | |
| CIDp780000976 | | 22 | | | |
| CIDp780000989 | | | | | |
| CIDp200796 | 28 | 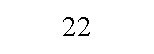 | 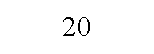 | 28 | 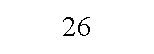 |
| CIDp201016 | 21 | 21 | 20 | 20 | 21 |
| | B | | | | |
| | TGFBeta Pathway activity scores of sepsis PBMC patients | | | | |
| | | | Vactosertib (1 uM) + | | R- |
| EXP 1 | | Vactosertib | R-bicalutamide | Etanercept | bicalutamide |
| TGFbeta | DMSO | (1 uM) | (25 uM) | (10 ug/ml) | (25 uM) |
| CIDp780000198 | 36 | 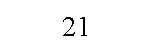 | 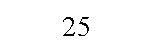 | 42 | 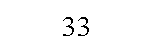 |
| CIDp780000197 | | | 36 | | |
| CIDp780000976 | | 31 | | | |
| CIDp780000989 | | | | | |
| CIDp200796 | 29 | 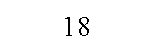 | 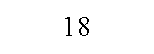 | 30 | 31 |
| CIDp201016 | 31 | 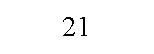 | 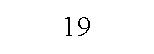 | 31 | 32 |
| | C | | | | |
| | AR Pathway activity scores of sepsis PBMC patients | | | | |
| EXP 2 | | Resatorvid | D4-abiraterone | A-485 | Galunisertib |
| AR | DMSO | (10 uM) | (10 uM) | (100 nM) | (10 uM) |
| CIDp780001003 | 25 | 24 | 24 | 24 | 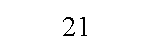 |
| CIDp780001079 | 26 | 31 | 33 | 27 | 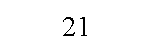 |
| CIDp780001144 | | | | | |
| CIDp520002787 | 24 | 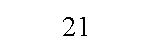 | 24 | 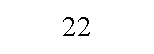 | 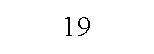 |
| CIDp780000128 | 23 | 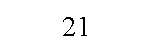 | 24 | 25 | 23 |
| CIDp780000130 | | | | | |
| | D | | | | |
| | TGFbeta Pathway activity scores of sepsis PBMC patients | | | | |
| EXP 2 | | Resatorvid | D4-abiraterone | A-485 | Galunisertib |
| TGFbeta | DMSO | (10 uM) | (10 uM) | (100 nM) | (10 uM) |
| CIDp780001003 | 29 | 31 | 32 | 28 | 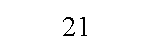 |
| CIDp780001079 | 31 | 37 | 41 | 33 | 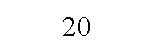 |
| CIDp780001144 | | | | | |
| CIDp520002787 | 36 | 35 | 36 | 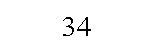 | 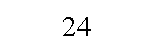 |
| CIDp780000128 | 32 | 34 | 40 | 35 | 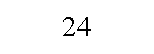 |
| CIDp780000130 | | | | | |
| | E | | | | |
| | AR Pathway activity scores of sepsis PBMC patients | | | | |
| | | D4- | | | D4- |
| | | abiraterone | R-bicalutamide | Vactosertib | abiraterone |
| | | (10 uM) + | (25 uM) + | (1 uM) + | (10 uM) + |
| | | Vactosertib | Etanercept (10 | Etanercept | Galunisertib |
| EXP 3 | DMSO | (1 uM) | ug/ml) | (10 ug/ml) | (10 uM) |
| CIDp780003612 | 20 | 21 | 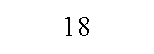 | 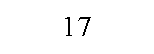 | 19 |
| CIDp780004319 | 19 | 21 | 19 | 21 | 21 |
| CIDp780001005 | | | | | |
| CIDp520002786 | 21 | 21 | 21 | 19 | 21 |
| CIDp780004317 | 21 | 20 | 21 | 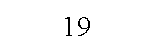 | 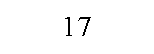 |
| CIDp780000196 | 32 | 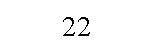 | 34 | 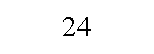 | 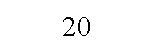 |
| | F | | | | |
| | TGFbeta Pathway activity scores of sepsis PBMC patients | | | | |
| | | D4- | R- | | |
| | | abiraterone | bicalutamide | Vactosertib | D4-abiraterone |
| | | (10 uM) + | (25 uM) + | (1 uM) + | (10 uM) + |
| | | Vactosertib | Etanercept (10 | Etanercept | Galunisertib |
| EXP 3 | DMSO | (1 uM) | ug/ml) | (10 ug/ml) | (10 uM) |
| CIDp780003612 | 27 | 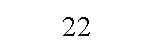 | 28 | 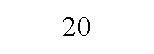 | 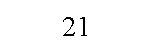 |
| CIDp780004319 | 30 | 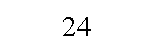 | 31 | 29 | 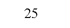 |
| CIDp780001005 | | | | | |
| CIDp520002786 | 32 | 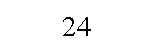 | 36 | 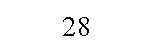 | 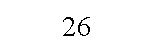 |
| CIDp780004317 | 29 | 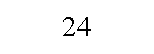 | 31 | 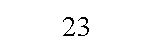 | 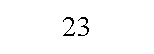 |
| CIDp780000196 | 37 | 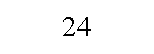 | 35 | 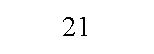 | 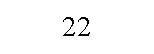 |

### REFERENCES

[1] N. L. Stanski and H. R. Wong, “Prognostic and predictive enrichment in sepsis,” Nature Reviews Nephrology. Nature Publishing Group, 1 Jan. 2019.
[2] “Recommendations|Sepsis: recognition, diagnosis and early management|Guidance NICE.”
[3] N. K. Patil, J. K. Bohannon, and E. R. Sherwood, “Immunotherapy: A promising approach to reverse sepsis-induced immunosuppression.,” Pharmacol. Res., vol. 111, pp. 688-702, 2016.
[4] R. S. Hotchkiss, G. Monneret, and D. Payen, “Sepsis-induced immunosuppression: from cellular dysfunctions to immunotherapy.,” Nat. Rev. Immunol., vol. 13, no. 12, pp. 862-74, Dec. 2013.
[5] M. R. Gubbels Bupp and T. N. Jorgensen, “Androgen-Induced Immunosuppression,” Front. Immunol., vol. 9, p. 794, Apr. 2018.
[6] A. Trigunaite and J. Dimo, “Suppressive effects of androgens on the immune system,” Cell. Immunol., vol. 294, no. 2, pp. 87-94, Apr. 2015.
[7] von Dipl Biochem Daniela Roll, “Androgen-regulation of sepsis response: Beneficial role of androgen receptor antagonists.”
[8] F. Fattahi and P. A. Ward, “Understanding Immunosuppression after Sepsis,” Nat. Rev. Mol. Cell Biol, vol. 42, pp. 51-65, 2017.
[9] A. Roquilly et al., “Local Modulation of Antigen-Presenting Cell Development after Resolution of Pneumonia Induces Long-Term Susceptibility to Secondary Infections,” Immunity, vol. 47, no. 1, pp. 135-147.e5, Jul. 2017.
[10] M. Cecconi et al., “Sepsis and septic shock”, Lancet 2018; 392: 75-87.
[11] Gubbels Bupp and Jorgensen, Androgen-Induced Immunosuppression, Front Immunol. 2018; 9: 794.
[12] Malinen et al., Crosstalk between androgen and pro-inflammatory signaling remodels androgen receptor and NF-κB cistrome to reprogram the prostate cancer cell transcriptome, Nucleic Acids Res. 2017 Jan. 25; 45(2): 619-630.
[13] Ben-Batalla et al., Influence of Androgens on Immunity to Self and Foreign: Effects on Immunity and Cancer, Front. Immunol., 2 Jul. 2020|https://doi.org/10.3389/fimmu.2020.01184.
[14] Sukhacheva, The role of monocytes in the progression of sepsis, Clinical Laboratory Int. 26 Aug. 2020.
[15] Haverman et al., The central role of monocytes in the pathogenesis of sepsis: consequences for immunomonitoring and treatment, The Netherlands Journal of Medicine, Volume 55, Issue 3, September 1999, Pages 132-141.
[16] Angele et al., Gender differences in sepsis, Virulence, 5:1, 12-19, DOI: 10.4161/viru.26982
[17]. Cecconi M, Evans L, Levy M, Rhodes A. Sepsis and septic shock. The Lancet. 2018 Jul. 7; 392(10141):75-87.
[18]. Lever A, Mackenzie I. Sepsis: definition, epidemiology, and diagnosis. BMJ. 2007 Oct. 27; 335(7625):879-83.
[19]. Singer M, Deutschman C S, Seymour C W, Shankar-Hari M, Annane D, Bauer M, et al. The Third International Consensus Definitions for Sepsis and Septic Shock (Sepsis-3). JAMA. 2016 Feb. 23; 315(8):801-10.
[20]. Newton K, Dixit V M. Signaling in innate immunity and inflammation. Cold Spring Harb Perspect Biol. 2012 Mar. 1; 4(3).
[21]. Cantrell D. Signaling in lymphocyte activation. Cold Spring Harb Perspect Biol. 2015 Jun. 1; 7(6).
[22]. Cantley editor of compilation.) Lewis C, Hunter editor of compilation.) Tony, Sever editor of compilation.) Richar, Thorner editor of compilation.) Jeremy W. Signal transduction: principles, pathways, and processes [Internet]. Cold Spring Harbor, New York: Cold Spring Harbor Laboratory Press; 2014 [cited 2018 Dec 2]. Available from: https://trove.nla.gov.au/version/203634108
[23]. Wesseling-Rozendaal Y, van Doom A, Willard-Gallo K, van de Stolpe A. Characterization of immunoactive and immunotolerant CD4+ T cells in breast cancer by measuring activity of signaling pathways that determine immune cell function [Internet]. Cancer Biology; 2020 Oct [cited 2021 Feb 24]. Available from: http://biorxiv.org/lookup/doi/10.1101/2020.10.08.292557
[24]. Bouwman W, Verhaegh W, Holtzer L, van de Stolpe A. Measurement of Cellular Immune Response to Viral Infection and Vaccination. Front Immunol. 2020; 11:575074.
[25]. Verhaegh W, Ooijen H van, Inda M A, Hatzis P, Versteeg R, Smid M, et al. Selection of Personalized Patient Therapy through the Use of Knowledge-Based Computational Models That Identify Tumor-Driving Signal Transduction Pathways. Cancer Res. 2014 Jun. 1; 74(11):2936-45.
[26]. van de Stolpe A, Holtzer L, van Ooijen H, Inda M A de, Verhaegh W. Enabling precision medicine by unravelling disease pathophysiology: quantifying signal transduction pathway activity across cell and tissue types. Scientific Reports. 2019 Feb. 7; 9(1):1603.
[27]. van Ooijen H, Hornsveld M, Dam-de Veen C, Velter R, Dou M, Verhaegh W, et al.

Assessment of Functional Phosphatidylinositol 3-Kinase Pathway Activity in Cancer Tissue Using Forkhead Box-O Target Gene Expression in a Knowledge-Based Computational Model. The American Journal of Pathology. 2018 Sep. 1; 188(9):1956-72.
[28]. Canté-Barrett K, Holtzer L, van Ooijen H, Hagelaar R, Cordo' V, Verhaegh W, et al. A Molecular Test for Quantifying Functional Notch Signaling Pathway Activity in Human Cancer. Cancers (Basel). 2020 Oct. 27; 12(11).
[29]. Frontiers|Measurement of Cellular Immune Response to Viral Infection and Vaccination Immunology [Internet]. [cited 2021 Feb 24]. Available from: https://www.frontiersin.org/articles/10.3389/fimmu.2020.575074/full
[30]. Bouwman W, Verhaegh W, Stolpe A van de. Androgen Receptor Pathway Activity Assay for Sepsis Diagnosis and Prediction of Favorable Prognosis. Frontiers in Medicine. 2021; 8:2352.
[31]. Bergmann C B, Beckmann N, Salyer C E, Hanschen M, Crisologo P A, Caldwell C C. Potential Targets to Mitigate Trauma- or Sepsis-Induced Immune Suppression. Front Immunol. 2021 Feb. 25; 12:622601.
[32]. Hiraki S, Ono S, Tsujimoto H, Kinoshita M, Takahata R, Miyazaki H, et al. Neutralization of interleukin-10 or transforming growth factor-β decreases the percentages of CD4+CD25+Foxp3+ regulatory T cells in septic mice, thereby leading to an improved survival. Surgery. 2012 Feb. 1; 151(2):313-22.
[33]. Peripheral Blood|Whole blood|Handbook|Miltenyi Biotec|Nederland [Internet]. [cited 2022 Feb 23]. Available from: https://www.miltenyibiotec.com/NL-en/resources/macs-handbook/human-cells-and-organs/human-cell-sources/blood-human.html#gref
[20]. The role of monocytes in the progression of sepsis—Clinical Laboratory int. [Internet]. [cited 2021 Feb 24]. Available from: https://clinlabint.com/the-role-of-monocytes-in-the-progression-of-sepsis/
[34]. Home-GEO DataSets-NCBI [Internet]. [cited 2021 Feb 24]. Available from: https://www.ncbi.nlm.nih.gov/gds/
[35]. Verhaegh W, van Ooijen H, Inda M A, Hatzis P, Versteeg R, Smid M, et al. Selection of personalized patient therapy through the use of knowledge-based computational models that identify tumor-driving signal transduction pathways. Cancer Res. 2014 Jun. 1; 74(11):2936-45.
[36]. van Ooijen H, Hornsveld M, Dam-de Veen C, Velter R, Dou M, Verhaegh W, et al. Assessment of Functional Phosphatidylinositol 3-Kinase Pathway Activity in Cancer Tissue Using Forkhead Box-O Target Gene Expression in a Knowledge-Based Computational Model. Am J Pathol. 2018 Sep; 188(9):1956-72.
[37]. Stolpe A van de, Holtzer L, van Ooijen H, de Inda M A, Verhaegh W. Enabling precision medicine by unravelling disease pathophysiology: quantifying signal transduction pathway activity across cell and tissue types. Sci Rep. 2019 Feb. 7; 9(1):1603.
[38]. Parman C, Halling C, Gentleman R. Parman C, Halling C, Gentleman R. AffyQCReport: QC report generation for affyBatch objects. R package version 1.58.0. 2018.
[39]. Gautier L, Cope L, Bolstad B M, Irizarry R A. affy-analysis of Affymetrix GeneChip data at the probe level. Bioinformatics. 2004 Feb. 12; 20(3):307-15.
[40]. Bicalutamide (ICI-176334)|>99% (HPLC) Selleck|Androgen Receptor antagonist [Internet]. selleckchem.com. [cited 2022 Jan 6]. Available from: https://www.selleckchem.com/products/Bicalutamide(Casodex).html
[41]. Bouwman W, Verhaegh W, Doom A van, Stolpe A van de. Measuring host immune response status by simultaneous quantitative measurement of activity of signal transduction pathways that coordinate functional activity of immune cells from innate and adaptive immune system [Internet]. bioRxiv; 2021 [cited 2022 Mar 7]. p. 2021.10.06.463309. Available from: https://www.biorxiv.com/content/10.1101/2021.10.06.463309v1

1. An AR cellular signaling pathway inhibitor for use in the prevention or treatment of sepsis in a subject, wherein the AR cellular signaling pathway inhibitor is administered if the AR cellular signaling pathway activity determined in the blood sample of the subject is found to be elevated or to exceed a certain threshold,
wherein the AR cellular signaling pathway inhibitor is selected from the group consisting of Steroidal antiandrogens, Nonsteroidal antiandrogens, Androgen synthesis inhibitors, CYP17A1 inhibitors, CYP11A1 (P450scc) inhibitors, 5α-Reductase inhibitors and Antigonadotropins or combinations thereof, or
wherein the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), and Progestogens (e.g., chlormadinone acetate, cyproterone acetate, gestonorone caproate, medroxyprogesterone acetate, megestrol acetate), etanercept, resatorvid, filgotinib or combinations thereof.

2. AR cellular signaling pathway inhibitor for use according to claim 1, wherein the use comprises:
determining the AR cellular signaling pathway activity in a blood sample of the subject; and
administering an AR cellular signaling pathway inhibitor to the patient if the AR cellular signaling pathway activity in the blood sample of the subject is found to be elevated or to exceed a certain threshold.

3. AR cellular signaling pathway inhibitor for use according to claim 2, wherein the determining the AR cellular signaling pathway comprises:
determining or receiving the expression levels of three or more target genes of the AR signaling pathway in the blood sample of the subject;
determining an activity level of the AR cellular signaling pathway associated transcription factor (TF) element in the sample, the TF element controlling transcription of the three or more target genes, the determining being based on evaluating a calibrated mathematical pathway model relating expression levels of the three or more target genes to the activity level of the AR cellular signaling pathway, and
inferring the activity of the AR cellular signaling pathway in the blood sample from the subject based on the determined activity level of the AR cellular signaling pathway associated TF element,
wherein the three or more target genes are selected from ABCC4, APP, AR, CDKN1A, CREB3L4, DHCR24, EAF2, ELL2, FGF8, FKBP5, GUCY1A3, IGF1, KLK2, KLK3, LCP1, LRIG1, NDRG1, NKX3_1, NTS, PLAU, PMEPA1, PPAP2A, PRKACB, PTPN1, SGK1, TACC2, TMPRSS2, and UGT2B15.

4. AR cellular signaling pathway inhibitor for use according to claim 1, wherein the AR cellular signaling pathway inhibitors is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, and Mifepristone (RU486), etanercept, resatorvid, filgotinib or combinations thereof.

5. AR cellular signaling pathway inhibitor for use according to claim 1, wherein the use is for the prevention of sepsis in a subject at risk of developing sepsis.

6. AR cellular signaling pathway inhibitor for use according to claim 1, wherein the use is for the treatment of sepsis in a subject suffering from sepsis.

7. AR cellular signaling pathway inhibitor for use according to claim 1, wherein the AR cellular signaling pathway inhibitor is administered together with a TGFbeta cellular signaling pathway inhibitor, wherein the AR cellular signaling pathway inhibitor and the TGFbeta cellular signaling pathway inhibitor are the same compound or a different compound.

8. AR cellular signaling pathway inhibitor for use according to claim 7, wherein the AR cellular signaling pathway inhibitor is administered prior to the TGFbeta cellular signaling pathway inhibitor, or wherein the AR cellular signaling pathway inhibitor is administered simultaneously with the TGFbeta cellular signaling pathway inhibitor, or wherein the AR cellular signaling pathway inhibitor is administered after the TGFbeta cellular signaling pathway inhibitor.

9. AR cellular signaling pathway inhibitor for use according to claim 1, wherein the use comprises:
determining the AR cellular signaling pathway activity and the TGFbeta cellular signaling pathway activity in a blood sample of the subject; and
administering an AR pathway inhibitor to the patient when the AR cellular signaling pathway activity is found to be elevated or to exceed a certain threshold; and
administering an TGFbeta pathway inhibitor to the patient when the TGFbeta cellular signaling pathway activity is found to be elevated or to exceed a certain threshold,
wherein the determining the TGFbeta cellular signaling pathway comprises:
determining or receiving the expression levels of three or more target genes of the TGFbeta signaling pathway in the blood sample of the subject;
determining an activity level of the TGFbeta cellular signaling pathway associated transcription factor (TF) element in the sample, the TF element controlling transcription of the three or more target genes, the determining being based on evaluating a calibrated mathematical pathway model relating expression levels of the three or more target genes to the activity level of the TGFbeta cellular signaling pathway, and
inferring the activity of the TGFbeta cellular signaling pathway in the blood sample from the subject based on the determined activity level of the TGFbeta cellular signaling pathway associated TF element,
wherein the three or more TGFbeta target genes are selected from ANGPTL4, CDC42EP3, CDKN1A, CDKN2B, CTGF, GADD45A, GADD45B, HMGA2, ID 1, I 1, INPP5D, JUNB, MMP2, MMP9, NKX2_5, OVOL1, PDGFB, PTHLH, SERPINE1, SGK1, SKIL, SMAD4, SMAD5, SMAD6, SMAD7, SNAI1, SNAI2, TIMP1 and VEGFA, and
wherein the TGFbeta cellular signaling pathway inhibitor is selected from the group consisting of Small molecule kinase inhibitors, Anti-TGF-P ligand antibodies, Anti-TOR receptor antibodies or Antisense oligonucleotides or combinations thereof, or
wherein the TGFbeta cellular signaling pathway inhibitor is selected from the group consisting of Vactosertib (EW-7197), Galunisertib (LY2157299), LY3200882, (E)-SIS3, Fresolimumab, XPA681, XPA089, LY2382770, LY3022859, ISTH0036, ISTH0047, Pyrrole-imidazole polyamides, etanercept, resatorvid, filgotinib or combinations thereof.

10. AR cellular signaling pathway inhibitor for use according to claim 7, wherein the TGFbeta cellular signaling pathway inhibitor is selected from the group consisting of Vactosertib (EW-7197), Galunisertib (LY2157299), LY3200882, (E)-SIS3, etanercept, resatorvid, filgotinib or combinations thereof.

11. A TGFbeta cellular signaling pathway inhibitor for use in the prevention or treatment of sepsis in a subject, wherein the TGFbeta cellular signaling pathway inhibitor is administered if the TGFbeta cellular signaling pathway activity determined in the blood sample of the subject is found to be elevated or to exceed a certain threshold,
wherein the TGFbeta cellular signaling pathway inhibitor is selected from the group consisting of Small molecule kinase inhibitors, Anti-TGF-β ligand antibodies, Anti-TOR receptor antibodies or Antisense oligonucleotides or combinations thereof, or
wherein the TGFbeta cellular signaling pathway inhibitor is selected from the group consisting of Vactosertib (EW-7197), Galunisertib (LY2157299), LY3200882, (E)-SIS3, Fresolimumab, XPA681, XPA089, LY2382770, LY3022859, ISTH0036, ISTH0047, Pyrrole-imidazole polyamides, etanercept, resatorvid, filgotinib or combinations thereof.

12. TGFbeta cellular signaling pathway inhibitor for use according to claim 11, wherein the use comprises:
determining the TGFbeta cellular signaling pathway activity in a blood sample of the subject; and
administering a TGFbeta cellular signaling pathway inhibitor to the patient if the TGFbeta cellular signaling pathway activity in the blood sample of the subject is found to be elevated or to exceed a certain threshold.

13. TGFbeta cellular signaling pathway inhibitor for use according to claim 12, wherein the determining the TGFbeta cellular signaling pathway comprises:
determining or receiving the expression levels of three or more target genes of the TGFbeta signaling pathway in the blood sample of the subject;
determining an activity level of the TGFbeta cellular signaling pathway associated transcription factor (TF) element in the sample, the TF element controlling transcription of the three or more target genes, the determining being based on evaluating a calibrated mathematical pathway model relating expression levels of the three or more target genes to the activity level of the TGFbeta cellular signaling pathway, and
inferring the activity of the TGFbeta cellular signaling pathway in the blood sample from the subject based on the determined activity level of the TGFbeta cellular signaling pathway associated TF element,
wherein the three or more TGFbeta target genes are selected from ANGPTL4, CDC42EP3, CDKN1A, CDKN2B, CTGF, GADD45A, GADD45B, HMGA2, ID 1, I 1, INPP5D, JUNB, MMP2, MMP9, NKX2_5, OVOL1, PDGFB, PTHLH, SERPINE1, SGK1, SKIL, SMAD4, SMAD5, SMAD6, SMAD7, SNAI1, SNAI2, TIMP1 and VEGFA.

14. TGFbeta cellular signaling pathway inhibitor for use according to claim 11, wherein the TGFbeta cellular signaling pathway inhibitor is selected from the group consisting of Vactosertib (EW-7197), Galunisertib (LY2157299), LY3200882, (E)-SIS3, etanercept, resatorvid, filgotinib or combinations thereof.

15. TGFbeta cellular signaling pathway inhibitor for use according to claim 11, wherein the TGFbeta cellular signaling pathway inhibitor is administered together with an AR cellular signaling pathway inhibitor, wherein the TGFbeta cellular signaling pathway inhibitor and the AR cellular signaling pathway inhibitor are the same compound or a different compound.

16. TGFbeta cellular signaling pathway inhibitor for use according to claim 15, wherein the TGFbeta cellular signaling pathway inhibitor is administered prior to the AR cellular signaling pathway inhibitor, or wherein the TGFbeta cellular signaling pathway inhibitor is administered simultaneously with the AR cellular signaling pathway inhibitor, or wherein the TGFbeta cellular signaling pathway inhibitor is administered after the AR cellular signaling pathway inhibitor.

17. An AR cellular signaling pathway inhibitor and TGFbeta cellular signaling pathway inhibitor for use in the prevention or treatment of sepsis in a subject,
wherein the TGFbeta cellular signaling pathway inhibitor and the AR cellular signaling pathway inhibitor are a different compound,
wherein the AR cellular signaling pathway inhibitor is selected from the group consisting of Steroidal antiandrogens, Nonsteroidal antiandrogens, Androgen synthesis inhibitors, CYP17A1 inhibitors, CYP11A1 (P450scc) inhibitors, 5α-Reductase inhibitors and Antigonadotropins or combinations thereof, or
wherein the AR cellular signaling pathway inhibitor is selected from the group consisting of Bicalutamide, (R)-Bicalutamide (HY-14249), Enzalutamide (MDV3100), N-desmethyl Enzalutamide, Proxalutamide (GT0918), Apalutamide (ARN-509), N-Desmethyl-Apalutamide, Darolutamide (ODM-201; BAY-1841788), Ketodarolutamide (ORM-15341), Galeterone (TOK-001), D4-abiraterone, A-485, Dexamethasone, Mifepristone (RU486), Cyproterone acetate, Chlormadinone acetate, Cyproterone acetate, Megestrol acetate, Osaterone acetate, Nomegestrol acetate, Dienogest, Oxendolone, Drospirenone, Spironolactone, Medrogestone, Bicalutamide, Flutamide, Nilutamide, Apalutamide, Cimetidine, Topilutamide, Abiraterone acetate, Ketoconazole, Seviteronel, Aminoglutethimide, Dutasteride, Alfatradiol, Dutasteride, Epristeride, Finasteride, A-485, ARCC-4, ARD-266, Saw palmetto extract, Leuprorelin, Estrogens (e.g., estradiol (and its esters), ethinylestradiol, conjugated estrogens, diethylstilbestrol), GnRH analogues, GnRH agonists (e.g., goserelin, leuprorelin), GnRH antagonists (e.g., cetrorelix), and Progestogens (e.g., chlormadinone acetate, cyproterone acetate, gestonorone caproate, medroxyprogesterone acetate, megestrol acetate), etanercept, resatorvid, filgotinib or combinations thereof, and
wherein the TGFbeta cellular signaling pathway inhibitor is selected from the group consisting of Small molecule kinase inhibitors, Anti-TGF-β ligand antibodies, Anti-TOR receptor antibodies or Antisense oligonucleotides or combinations thereof, or
wherein the TGFbeta cellular signaling pathway inhibitor is selected from the group consisting of Vactosertib (EW-7197), Galunisertib (LY2157299), LY3200882, (E)-SIS3, Fresolimumab, XPA681, XPA089, LY2382770, LY3022859, ISTH0036, ISTH0047, Pyrrole-imidazole polyamides, etanercept, resatorvid, filgotinib or combinations thereof.
